SEQUENCE LISTING (3 sequences)

SEQ ID NO: 1 - protein, 200 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQ

SEQ ID NO: 2 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 3 - protein, 151 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

(19) United States
(12) Patent Application Publication
(10) Pub. No.: US 2025/0298025 A1
(43) Pub. Date: Sep. 25, 2025

(54) METHODS FOR THE DETECTION AND TREATMENT OF PANCREATIC DUCTAL ADENOCARCINOMA

(71) Applicant: Board of Regents, The University of Texas System, Austin, TX (US)

(72) Inventors: Samir HANASH, Houston, TX (US); Michela CAPELLO, Houston, TX (US); Ayumu TAGUCHI, Houston, TX (US); Ziding FENG, Houston, TX (US)

(21) Appl. No.: 19/065,894

(22) Filed: Feb. 27, 2025

Related U.S. Application Data
(63) Continuation of application No. 18/054,307, filed on Nov. 10, 2022, now abandoned.
Application No. 18/054,307 is a continuation of application No. 16/469,065, filed on Jun. 12, 2019, now abandoned.
(60) Provisional application No. 62/435,020, filed on Dec. 15, 2016.
(60) Provisional application No. 62/435,024, filed on Dec. 15, 2016.

(51) Int. Cl. G01N 33/574; G01N 30/72; H01J 49/00

(52) U.S. Cl. CPC G01N 33/57438; H01J 49/0027; G01N 30/7233; G01N 2333/8146; G01N 2400/00

(57) ABSTRACT

Provided are methods and related kits for detection of early stage pancreatic ductal adenocarcinoma. Also provided are methods for treating a patient susceptible, or suspected of being susceptible, to pancreatic ductal adenocarcinoma.

### CROSS REFERENCE TO RELATED APPLICATIONS

This application is a continuation of U.S. application Ser. No. 16/469,065, filed Jun. 12, 2019, which is a national stage entry under 35 U.S.C. § 371 of International Application No. PCT/US2017/066851, filed Dec. 15, 2017, which claims the benefit of U.S. Provisional Application No. 62/435,024, and U.S. Provisional Application No. 62/435,020, both of which were filed Dec. 15, 2016, the disclosures of each are hereby incorporated by reference in their entireties.

### STATEMENT REGARDING FEDERALLY SPONSORED RESEARCH OR DEVELOPMENT

This invention was made with government support under grant number CA141550 awarded by the National Institutes of Health. The government has certain rights in the invention.

### INCORPORATION OF SEQUENCE LISTING

The sequence listing that is contained in the file named “MDA0024-505C2-US.xml,” which is 4.19 kilobytes as measured in Microsoft Windows operating system and was created on Nov. 10, 2022, is filed electronically herewith and incorporated herein by reference.

### BACKGROUND

Pancreatic ductal adenocarcinoma (PDAC) is one of the most lethal types of cancer with a 5-year survival rate of only 8% and a mortality rate closely approaching the incidence rate. Although resectable PDAC is associated with better survival, only 15-20% of PDAC patients present with localized disease. Imaging modalities, notably endoscopic ultrasound and magnetic resonance cholangiopancreatography, are currently used in the work up of subjects with suspected PDAC or at high risk for the disease. However, known risk factors have only a modest effect on PDAC incidence.

Cancer Antigen 19-9 (CA19-9) is currently in clinical use as a PDAC biomarker. CA19-9 has shown potential as a diagnostic biomarker for both preclinical and early-stage PDAC (Riker et al., Surgical Oncology 6:157-69, 1998). However, CA19-9 alone has limited performance as a biomarker for early-stage disease: less than 75% of pancreatic cancer patients present with elevated CA19-9, and many benign disorders can lead to elevated CA19-9 levels. Moreover, CA19-9 is not detectable in 5-10% of patients with fucosyltransferase deficiency and inability to synthesize antigens of the Lewis blood group. As such, the proportions of individuals incorrectly identified as having PDAC, as well as those incorrectly identified as not having PDAC, is unacceptably high for reliance on CA19-9 alone as a diagnostic tool.

Due to late diagnosis, growing incidence, and limited avenues of treatment, PDAC is set to become a leading cause of cancer-related death. Given the disease is generally diagnosed in an advanced stage in most patients, and use of CA19-9 as a standalone biomarker is clearly inadequate, there is a need to develop a test for the detection of pancreatic cancer at an early stage.

### SUMMARY

The present disclosure provides methods and kits for the early detection of pancreatic cancer. The methods and kits use multiple assays of biomarkers contained within a biological sample obtained from a subject. The combined analysis of at least three biomarkers: carbohydrate antigen 19-9 (CA19-9), TIMP metallopeptidase inhibitor 1 (TIMP1), and leucine-rich alpha-2-glycoprotein 1 (LRG1), provides high-accuracy diagnosis of PDAC when screened against cohorts with known status.

In some embodiments, the analysis of biomarkers CA19-9, TIMP1, and LRG1, can be combined with analysis of additional biomarkers. In some embodiments, the additional biomarkers can be protein biomarkers. In some embodiments, the additional protein biomarkers can be selected from the group consisting of ALCAM, CHI3L1, COL18A1, IGFBP2, LCN2, LYZ, PARK7, REG3A, SLPI, THBS1, TNFRSF1A, WFDC2, and any combination thereof. In some embodiments, the additional biomarkers can be non-protein biomarkers. In some embodiments, the non-protein biomarkers can be circulating tumor DNA (ctDNA). In some embodiments, a method as described herein may further comprise: measuring the level of (N1/N8)-acetylspermidine (AcSperm) in the biological sample; measuring the level of diacetylspermine (DAS) in the biological sample; measuring the level of lysophosphatidylcholine (LPC) (18:0) in the biological sample; measuring the level of lysophosphatidylcholine (LPC) (20:3) in the biological sample; and measuring the level of an indole-derivative in the biological sample; wherein the amount of (N1/N8)-acetylspermidine (AcSperm), diacetylspermine (DAS), lysophosphatidylcholine (LPC) (18:0), lysophosphatidylcholine (LPC) (20:3), and the indole-derivative classifies the patient as being susceptible to pancreatic ductal adenocarcinoma or not susceptible to pancreatic ductal adenocarcinoma.

A regression model was identified that can predict the PDAC status for a subject based on levels of CA19-9, TIMP1, and LRG1 found in a biological sample from the subject.

In some embodiments, biomarkers are measured in blood samples drawn from patients. In some embodiments, the presence or absence of biomarkers in a biological sample can be determined. In some embodiments, the level of biomarkers in a biological sample can be quantified.

In some embodiments, a surface is provided to analyze a biological sample. In some embodiments, biomarkers of interest adsorb nonspecifically onto this surface. In some embodiments, receptors specific for biomarkers of interest are incorporated onto this surface.

In some embodiments, the surface is associated with a particle, for example, a bead. In some embodiments, the surface is contained in a multi-well plate to facilitate simultaneous measurements.

In some embodiments, multiple surfaces are provided for parallel assessment of biomarkers. In some embodiments, the multiple surfaces are provided on a single device, for example a 96-well plate. In some embodiments, the multiple surfaces enable simultaneous measurement of biomarkers. In some embodiments, a single biological sample can be applied sequentially to a plurality of surfaces. In some embodiments, a biological sample is divided for simultaneous application to a plurality of surfaces.

In some embodiments, the biomarker binds to a particular receptor molecule, and the presence or absence of the biomarker-receptor complex can be determined. In some embodiments, the amount of biomarker-receptor complex can be quantified. In some embodiments, the receptor molecule is linked to an enzyme to facilitate detection and quantification.

In some embodiments, the biomarker binds to a particular relay molecule, and the biomarker-relay molecule complex in turn binds to a receptor molecule. In some embodiments, the presence or absence of the biomarker-relay-receptor complex can be determined. In some embodiments, the amount of biomarker-relay-receptor complex can be quantified. In some embodiments, the receptor molecule is linked to an enzyme to facilitate detection and quantification. In some embodiments, the enzyme is horseradish peroxidase or alkaline phosphatase.

In some embodiments, a biological sample is analyzed sequentially for individual biomarkers. In some embodiments, a biological sample is divided into separate portions to allow for simultaneous analysis for multiple biomarkers. In some embodiments, a biological sample is analyzed in a single process for multiple biomarkers.

In some embodiments, the absence or presence of biomarker can be determined by visual inspection. In some embodiments, the quantity of biomarker can be determined by use of a spectroscopic technique. In some embodiments, the spectroscopic technique is mass spectrometry. In some embodiments, the spectroscopic technique is UV/Vis spectrometry. In some embodiments, the spectroscopic technique is an excitation/emission technique such as fluorescence spectrometry.

In some embodiments, a kit is provided for analysis of a biological sample. In some embodiments, the kit can contain chemicals and reagents required to perform the analysis. In some embodiments, the kit contains a means for manipulating biological samples in order to minimize the required operator intervention.

In another aspect, the disclosure provides a method of determining susceptibility of a patient to pancreatic ductal adenocarcinoma, comprising obtaining a biological sample from the patient; measuring the level of (N1/N8)-acetylspermidine (AcSperm) in the biological sample; measuring the level of diacetylspermine (DAS) in the biological sample; measuring the level of lysophosphatidylcholine (LPC) (18:0) in the biological sample; measuring the level of lysophosphatidylcholine (LPC) (20:3) in the biological sample; and measuring the level of an indole-derivative in the biological sample; wherein the amount of (N1/N8)-acetylspermidine (AcSperm), diacetylspermine (DAS), lysophosphatidylcholine (LPC) (18:0), lysophosphatidylcholine (LPC) (20:3), and the indole-derivative classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising a plasma-derived biomarker panel and a protein marker panel: wherein the plasma-derived biomarker panel comprises (N1/N8)-acetylspermidine (AcSperm), diacetylspermine (DAS), lysophosphatidylcholine (LPC) (18:0), lysophosphatidylcholine (LPC) (20:3), and an indole-derivative; wherein the protein biomarker panel comprises CA19-9, LRG1, and TIMP1; wherein the method comprises: obtaining a biological sample from the patient; measuring the levels of the plasma-derived biomarkers and the protein biomarkers in the biological sample; wherein the amount of the plasma-derived biomarkers and the protein biomarkers classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising determining the levels of one or more protein biomarkers and one or more metabolite markers, said method comprising: obtaining a biological sample from the patient; contacting the sample with a first reporter molecule that binds CA19-9 antigen; contacting the sample with a second reporter molecule that binds TIMP1 antigen; contacting the sample with a third reporter molecule that binds LRG1 antigen; and determining the levels of the one or more biomarkers, wherein the one or more biomarkers is selected from the group consisting of (N1/N8)-acetylspermidine (AcSperm), diacetylspermine (DAS), lysophosphatidylcholine (LPC) (18:0), lysophosphatidylcholine (LPC) (20:3), and an indole-derivative; wherein the amount of the first reporter molecule, the second reporter molecule, the third reporter molecule, and the one or more biomarkers classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising obtaining a biological sample from the patient; measuring the levels of CA19-9, TIMP1, and LRG1 antigens in the biological sample; and measuring the levels of one or more metabolite markers selected from the group consisting of (N1/N8)-acetylspermidine (AcSperm), diacetylspermine (DAS), lysophosphatidylcholine (LPC) (18:0), lysophosphatidylcholine (LPC) (20:3), and an indole-derivative in the biological sample; assigning the condition of the patient as either susceptible to pancreatic ductal adenocarcinoma or not susceptible to pancreatic ductal adenocarcinoma, as determined by statistical analysis of the levels of CA19-9 antigen, TIMP1 antigen, LRG1 antigen, (N1/N8)-acetylspermidine (AcSperm), diacetylspermine (DAS), lysophosphatidylcholine (LPC) (18:0), lysophosphatidylcholine (LPC) (20:3), and the indole-derivative in the biological sample.

In another aspect, the disclosure provides a method of treating a patient suspected of susceptibility to pancreatic ductal adenocarcinoma, comprising: analyzing the patient for susceptibility to pancreatic ductal adenocarcinoma with a method as recited in any one of claims 38-41; administering a therapeutically effective amount of a treatment for the adenocarcinoma. In one embodiment, the treatment is surgery, chemotherapy, radiation therapy, targeted therapy, or a combination thereof.

In one embodiment, a method as described herein comprises at least one receptor molecule that selectively binds to an antigen selected from the group consisting of CA19-9, TIMP1, and LRG1.

In one embodiment, detection of the amount of CA19-9, TIMP1, LRG, (N1/N8)-acetylspermidine (AcSperm), diacetylspermine (DAS), lysophosphatidylcholine (LPC) (18:0), lysophosphatidylcholine (LPC) (20:3), or the indole-derivative comprises the use of a solid particle. In another embodiment, the solid particle is a bead.

In one embodiment, at least one of the reporter molecules is linked to an enzyme.

In one embodiment, at least one of the protein or metabolite markers generates a detectable signal. In another embodiment, the detectable signal is detectable by a spectrometric method. In another embodiment, the spectrometric method is mass spectrometry.

In one embodiment, a method as described herein comprises inclusion of patient history information into the assignment of having pancreatic ductal adenocarcinoma or not having pancreatic ductal adenocarcinoma.

In one embodiment, a method as described herein comprises administering at least one alternate diagnostic test for a patient assigned as having pancreatic ductal adenocarcinoma. In another embodiment, the at least one alternate diagnostic test comprises an assay or sequencing of at least one ctDNA.

In another aspect, the disclosure provides a kit for a method as described herein, comprising: a reagent solution that comprises a first solute for detection of CA19-9 antigen; a second solute for detection of LRG1 antigen; a third solute for detection of TIMP1 antigen; a fourth solute for detection of (N1/N8)-acetylspermidine (AcSperm); a fifth solute for detection of diacetylspermine (DAS); a sixth solute for detection of lysophosphatidylcholine (LPC) (18:0); a seventh solute for detection of lysophosphatidylcholine (LPC) (20:3); and an eighth solute for detection of the indole-derivative.

In one embodiment, such a kit may comprise a first reagent solution that comprises a first solute for detection of CA19-9 antigen; a second reagent solution that comprises a second solute for detection of LRG1 antigen; a third reagent solution that comprises a third solute for detection of TIMP1 antigen; a fourth reagent solution that comprises a fourth solute for detection of (N1/N8)-acetylspermidine (AcSperm); a fifth reagent solution that comprises a fifth solute for detection of diacetylspermine (DAS); a sixth reagent solution that comprises a sixth solute for detection of lysophosphatidylcholine (LPC) (18:0); a seventh reagent solution that comprises a seventh solute for detection of lysophosphatidylcholine (LPC) (20:3); and an eighth reagent solution that comprises an eighth solute for detection of the indole-derivative.

In one embodiment, a kit as described herein may comprise a device for contacting the reagent solutions with a biological sample. In another embodiment, such a kit may comprise at least one surface with means for binding at least one antigen. In another embodiment, the at least one antigen is selected from the group consisting of CA19-9, LRG1, and TIMP1. In another embodiment, the at least one surface comprises a means for binding ctDNA.

In another aspect, the disclosure provides such a method as described herein wherein the method further comprises: measuring the level of (N1/N8)-acetylspermidine (AcSperm) in the biological sample; measuring the level of diacetylspermine (DAS) in the biological sample; measuring the level of lysophosphatidylcholine (LPC) (18:0) in the biological sample; measuring the level of lysophosphatidylcholine (LPC) (20:3) in the biological sample; and measuring the level of an indole-derivative in the biological sample; wherein the amount of (N1/N8)-acetylspermidine (AcSperm), diacetylspermine (DAS), lysophosphatidylcholine (LPC) (18:0), lysophosphatidylcholine (LPC) (20:3), and the indole-derivative classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising obtaining a biological sample from the patient; measuring the level of (N1/N8)-acetylspermidine (AcSperm) in the biological sample; measuring the level of diacetylspermine (DAS) in the biological sample; measuring the level of lysophosphatidylcholine (LPC) (18:0) in the biological sample; measuring the level of lysophosphatidylcholine (LPC) (20:3) in the biological sample; and measuring the level of an indole-derivative in the biological sample; wherein the amount of (N1/N8)-acetylspermidine (AcSperm), diacetylspermine (DAS), lysophosphatidylcholine (LPC) (18:0), lysophosphatidylcholine (LPC) (20:3), and the indole-derivative classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising a plasma-derived biomarker panel and a protein marker panel: wherein the plasma-derived biomarker panel comprises (N1/N8)-acetylspermidine (AcSperm), diacetylspermine (DAS), lysophosphatidylcholine (LPC) (18:0), lysophosphatidylcholine (LPC) (20:3), and an indole-derivative; wherein the protein biomarker panel comprises CA19-9, LRG1, and TIMP1; wherein the method comprises: obtaining a biological sample from the patient; measuring the levels of the plasma-derived biomarkers and the protein biomarkers in the biological sample; wherein the amount of the plasma-derived biomarkers and the protein biomarkers classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising determining the levels of one or more protein biomarkers and one or more metabolite markers, said method comprising: obtaining a biological sample from the patient; contacting the sample with a first reporter molecule that binds CA19-9 antigen; contacting the sample with a second reporter molecule that binds TIMP1 antigen; contacting the sample with a third reporter molecule that binds LRG1 antigen; and determining the levels of the one or more biomarkers, wherein the one or more biomarkers is selected from the group consisting of (N1/N8)-acetylspermidine (AcSperm), diacetylspermine (DAS), lysophosphatidylcholine (LPC) (18:0), lysophosphatidylcholine (LPC) (20:3), and an indole-derivative; wherein the amount of the first reporter molecule, the second reporter molecule, the third reporter molecule, and the one or more biomarkers classifies the patient as being susceptible to pancreatic ductal adenocarcinoma or not susceptible to pancreatic ductal adenocarcinoma.

In another aspect, the disclosure provides a method of determining susceptibility of a patient to pancreatic ductal adenocarcinoma, comprising obtaining a biological sample from the patient; measuring the levels of CA19-9, TIMP1, and LRG1 antigens in the biological sample; and measuring the levels of one or more metabolite markers selected from the group consisting of (N1/N8)-acetylspermidine (AcSperm), diacetylspermine (DAS), lysophosphatidylcholine (LPC) (18:0), lysophosphatidylcholine (LPC) (20:3), and an indole-derivative in the biological sample; assigning the condition of the patient as either susceptible to pancreatic ductal adenocarcinoma or not susceptible to pancreatic ductal adenocarcinoma, as determined by statistical analysis of the levels of CA19-9 antigen, TIMP1 antigen, LRG1 antigen, (N1/N8)-acetylspermidine (AcSperm), diacetylspermine (DAS), lysophosphatidylcholine (LPC) (18:0), lysophosphatidylcholine (LPC) (20:3), and the indole-derivative in the biological sample.

In another aspect, the disclosure provides a method of treating a patient suspected of susceptibility to pancreatic ductal adenocarcinoma, comprising: analyzing the patient for susceptibility to pancreatic ductal adenocarcinoma with a method as recited in any one of claims 36-39; administering a therapeutically effective amount of a treatment for the adenocarcinoma. In one embodiment, the treatment is surgery, chemotherapy, radiation therapy, targeted therapy, or a combination thereof. In another embodiment, such a method comprises at least one receptor molecule that selectively binds to an antigen selected from the group consisting of CA19-9, TIMP1, and LRG1. In another embodiment, detection of the amount of CA19-9, TIMP1, LRG, (N1/N8)-acetylspermidine (AcSperm), diacetylspermine (DAS), lysophosphatidylcholine (LPC) (18:0), lysophosphatidylcholine (LPC) (20:3), or the indole-derivative comprises the use of a solid particle. In another embodiment, the solid particle is a bead. In another embodiment, at least one of the reporter molecules is linked to an enzyme. In another embodiment, at least one of the protein or metabolite markers generates a detectable signal. In another embodiment, the detectable signal is detectable by a spectrometric method. In another embodiment, the spectrometric method is mass spectrometry. In another embodiment, such a method comprises inclusion of patient history information into the assignment of having pancreatic ductal adenocarcinoma or not having pancreatic ductal adenocarcinoma. In another embodiment, such a method comprises administering at least one alternate diagnostic test for a patient assigned as having pancreatic ductal adenocarcinoma. In another embodiment, the at least one alternate diagnostic test comprises an assay or sequencing of at least one ctDNA.

In another aspect, the disclosure provides a kit for the method as recited in any one of claims 36-40, comprising: a reagent solution that comprises a first solute for detection of CA19-9 antigen; a second solute for detection of LRG1 antigen; a third solute for detection of TIMP1 antigen; a fourth solute for detection of (N1/N8)-acetylspermidine (AcSperm); a fifth solute for detection of diacetylspermine (DAS); a sixth solute for detection of lysophosphatidylcholine (LPC) (18:0); a seventh solute for detection of lysophosphatidylcholine (LPC) (20:3); and an eighth solute for detection of the indole-derivative. In another embodiment, a kit as disclosed herein comprises a first reagent solution that comprises a first solute for detection of CA19-9 antigen; a second reagent solution that comprises a second solute for detection of LRG1 antigen; a third reagent solution that comprises a third solute for detection of TIMP1 antigen; a fourth reagent solution that comprises a fourth solute for detection of (N1/N8)-acetylspermidine (AcSperm); a fifth reagent solution that comprises a fifth solute for detection of diacetylspermine (DAS); a sixth reagent solution that comprises a sixth solute for detection of lysophosphatidylcholine (LPC) (18:0); a seventh reagent solution that comprises a seventh solute for detection of lysophosphatidylcholine (LPC) (20:3); and an eighth reagent solution that comprises an eighth solute for detection of the indole-derivative. In one embodiment, such a kit comprises a device for contacting the reagent solutions with a biological sample. In another embodiment, such a kit comprises at least one surface with means for binding at least one antigen. In another embodiment, the at least one antigen is selected from the group consisting of CA19-9, LRG1, and TIMP1. In another embodiment, the at least one surface comprises a means for binding ctDNA.

In another aspect, the disclosure provides a method of treatment or prevention of progression of pancreatic ductal adenocarcinoma (PDAC) in a patient in whom the levels of CA19-9 antigen, TIMP1 antigen, and LRG1 antigen classifies the patient as having or being susceptible to PDAC comprising one or more of: administering a chemotherapeutic drug to the patient with PDAC; administering therapeutic radiation to the patient with PDAC; and surgery for partial or complete surgical removal of cancerous tissue in the patient with PDAC. In ne embodiment, the levels of CA19-9 antigen, TIMP1 antigen, and LRG1 antigen are elevated. In another embodiment, the levels of CA19-9 antigen, TIMP1 antigen, and LRG1 antigen are elevated in comparison to the levels of CA19-9 antigen, TIMP1 antigen, and LRG1 antigen in a reference patient or group that does not have PDAC. In another embodiment, the reference patient or group is healthy. In another embodiment, the AUC (95% CI) is at least 0.850. In another embodiment, the AUC (95% CI) is at least 0.900. In another embodiment, the classification of the patient as having PDAC has a sensitivity of 0.849 and 0.658 at 95% and 99% specificity, respectively. In another embodiment, the levels of CA19-9 antigen, TIMP1 antigen, and LRG1 antigen are elevated in comparison to the levels of CA19-9 antigen, TIMP1 antigen, and LRG1 antigen in a reference patient or group that has chronic pancreatitis. In another embodiment, the levels of CA19-9 antigen, TIMP1 antigen, and LRG1 antigen are elevated in comparison to the levels of CA19-9 antigen, TIMP1 antigen, and LRG1 antigen in a reference patient or group that has benign pancreatic disease. In another embodiment, the AUC (95% CI) is at least 0.850. In another embodiment, the AUC (95% CI) is at least 0.900. In another embodiment, the classification of the patient as having PDAC has a sensitivity of 0.849 and 0.658 at 95% and 99% specificity, respectively. In another embodiment, the PDAC is diagnosed at or before the borderline resectable stage. In another embodiment, the PDAC is diagnosed at the resectable stage.

In another aspect, the disclosure provides a method of treatment or prevention of progression of pancreatic ductal adenocarcinoma (PDAC) in a patient in whom the levels of CA19-9 antigen, TIMP1 antigen, LRG1, N1/N8)-acetylspermidine (AcSperm), diacetylspermine (DAS), lysophosphatidylcholine (LPC) (18:0), lysophosphatidylcholine (LPC) (20:3), and an indole-derivative classifies the patient as having or being susceptible to PDAC comprising one or more of: administering a chemotherapeutic drug to the patient with PDAC; administering therapeutic radiation to the patient with PDAC; and surgery for partial or complete surgical removal of cancerous tissue in the patient with PDAC. In another embodiment, the levels of CA19-9 antigen, TIMP1 antigen, and LRG1 antigen are elevated. In another embodiment, the levels of CA19-9 antigen, TIMP1 antigen, and LRG1 antigen are elevated in comparison to the levels of CA19-9 antigen, TIMP1 antigen, and LRG1 antigen in a reference patient or group that does not have PDAC. In another embodiment, the reference patient or group is healthy. In another embodiment, the levels of CA19-9 antigen, TIMP1 antigen, and LRG1 antigen are elevated in comparison to the levels of CA19-9 antigen, TIMP1 antigen, and LRG1 antigen in a reference patient or group that has chronic pancreatitis. In another embodiment, the levels of CA19-9 antigen, TIMP1 antigen, and LRG1 antigen are elevated in comparison to the levels of CA19-9 antigen, TIMP1 antigen, and LRG1 antigen in a reference patient or group that has benign pancreatic disease. In another embodiment, the patient is at high-risk of PDAC. In another embodiment, the patient is over age 50 years with new-onset diabetes mellitus, has chronic pancreatitis, has been incidentally diagnosed with mucin-secreting cysts of the pancreas, or is asymptomatic kindred of one of these high-risk groups.

In another aspect, the disclosure provides a method of treating a patient suspected of susceptibility to pancreatic ductal adenocarcinoma, comprising analyzing the patient for susceptibility to pancreatic ductal adenocarcinoma with a method as described herein; administering a therapeutically effective amount of a treatment for the adenocarcinoma. In another embodiment, the treatment is surgery, chemotherapy, radiation therapy, targeted therapy, or a combination thereof.

### BRIEF DESCRIPTION OF THE DRAWINGS

FIG. 1 depicts a flow chart for discovery of the validated biomarker model.

FIG. 2A and FIG. 2B depict biomarker candidates with significantly higher levels in PDAC than healthy controls in the triage set. Performance of the biomarker candidates in the comparison of (FIG. 2A) PDAC (n=75) versus healthy controls (n=27) and (FIG. 2B) PDAC versus chronic pancreatitis patients (n=19) in the triage set. Bars indicate AUC (95% CI). *Indicates that the reverse ordering was used. AUC, area under the curve.

FIG. 3A and FIG. 3B depict performance of the biomarker panel based on TIMP1+LRG1+CA19-9 in the combined validation set. ROC analysis of the biomarker panel developed for (FIG. 3A) PDAC versus healthy control and (FIG. 3B) PDAC versus benign pancreatic disease (“OR” rule combination). Upper line shows the model, and lower line shows CA19-9. AUC, area under the curve.

FIG. 4 depicts a correlation analysis between the biomarker panel (TIMP1, LRG1, and CA19-9) based scores and tumor size values in validation set #2. The underlying linear regression model yields intercept and slope of 3.7329 and −0.2646, respectively (slope 95% CI=−0.745-0.216; Wald-based two-sided p-value=0.27). Tumor size refers to the larger of the two measurements assessed by CT/MRI/EUS.

FIG. 5 depicts performance of the biomarker model based on TIMP1+LRG1+CA19-9 in the test set. ROC analysis of the combination model with fixed coefficients, which was developed in combined validation sets, for PDAC versus healthy control. Upper line shows the model, and lower line shows CA19-9. AUC, area under the curve.

FIG. 6 depicts a schematic of study design and filtering strategy.

FIG. 7 depicts individual AUCs for detected lysophosphatidylcholines, sphingomyelins and ceramides in the discovery cohort. Abbrev: LPC: lysophosphatidycholine; SM: sphingomyelins.

FIG. 8 depicts MSMS spectra for indole-derivative; matched fragments occur at about 118, about 148, and about 188 m/z.

FIG. 9A and FIG. 9B depict AUC curves of individual metabolites and 5-marker metabolite panel in the Training Sets. Performances are based on the combined discovery and ‘confirmatory’ cohort. (FIG. 9A) Receiver operating characteristic (ROC) curves for individual metabolites and the 5-marker metabolite panel for distinguishing PDAC (n=29) from healthy subjects (n=10). (FIG. 9B) ROC curves for individual metabolites and the 5-marker metabolite panel comparing PDAC (n=29) relative to subjects diagnosed with benign pancreatic disease (chronic pancreatitis (n=10) and low-grade cysts (n=50)).

FIG. 10A and FIG. 10B depict validation of individual metabolites and the 5-marker metabolite panel in Test Sets. (FIG. 10A) Receiver operating characteristic (ROC) curves for individual metabolites and the 5-marker metabolite panel for distinguishing resectable PDAC (n=39) from healthy subjects (n=82) (Test Set #1). (FIG. 10B) ROC curves for individual metabolites and the 5-marker metabolite panel comparing resectable PDAC (n=20) relative to subjects diagnosed with benign pancreatic disease (low-grade cysts (n=102)) (Test Set #2).

FIG. 11A and FIG. 11B depict a hyper-panel consisting of a metabolite-panel and a protein-panel improves classification as compared to protein-panel alone. (FIG. 11A and FIG. 11B) ROC Curves for hyper-panel and protein-panel only in the Training Set (29 PDAC vs 10 healthy subjects) and independent validation cohort (Test Set #1; 39 PDAC vs 82 healthy subjects).

FIG. 12A-FIG. 12D depict pancreatic ductal adenocarcinomas catabolize extracellular lysophospholipids. (FIG. 12A) Percentage (%) change in serum-containing media composition of lysophosphatidylcholine (18:0), lysophosphatidylcholine (20:3), and glycerophosphocholine in PANC1 and SU8686 PDAC cell lines following 24, 48, and 72 hours of culturing. (FIG. 12B) Schematic illustrating enzymes involved in catabolism of phosphatidylcholines and lysophosphatidylcholines. (FIG. 12C) mRNA expression+/−SEM of PLA2G10, LYPLA1, and ENPP2 in PDAC and adjacent control tissue. Statistical significance was determined by paired t-test (p: **<0.01, ****<0.001). mRNA expression data was obtained from Oncomine and is based on the Badea dataset (FIG. 12D).

FIG. 13 depicts composition of lipid species in conditioned media. Heatmap depicting % change in composition of lipid species in 24, 48, and 72-hour conditioned serum-containing media from PDAC cell lines PANC-1 and SU8686 as compared to media blank. Abbrev: PC: phosphatidylcholine; PE: phosphatidylethanolamine; LPC: lysophosphatidycholine; LPE: lysophosphatidylethanolamine; Plas: Plasmalogen.

FIG. 14A-FIG. 14C depict pancreatic ductal adenocarcinomas exhibit elevated catabolism of polyamines. (FIG. 14A) Abundances (area units+/−stdev) of N1/N8-acetylspermidine or diacetylspermine in cell lysates of 5 PDAC cell lines (CFPAC-1, MiaPaCa, SU8686, PANC03-27, and SW1990). (FIG. 14B) Abundance (area units+/−stdev) of N1/N8-acetylspermidine or diacetylspermine in serum free media collected 1, 2, 4, and 6 hours post conditioning from 5 PDAC cell lines (CFPAC-1, MiaPaCa, SU8686, PANC03-27, and SW1990). (FIG. 14C) Network displaying enzymes involved in the biosynthesis of polyamines and their acetylated derivatives. Node shade (light gray=decreased; dark gray=increased) and size depicts direction and magnitude of change in mRNA expression of respective enzymes between PDAC and adjacent control tissue. Thickened node border illustrates statistical significance (paired t-test<0.05). Box and whisker plots illustrate distribution of mRNA expression for the respective enzyme between PDAC and adjacent control tissue. mRNA expression data was obtained from Oncomine and is based on the Badea dataset.

### DETAILED DESCRIPTION

Provided are methods for identifying pancreatic cancer in a human subject, the methods generally comprising:
(a) applying a blood sample obtained from the subject to an assay for analysis of at least three biomarkers: CA19-9, TIMP1, and LRG1;
(b) quantifying the amount of the at least three biomarkers present in the blood sample; and
(c) applying statistical analysis based on the amount of biomarkers present to determine a biomarker score with respect to corresponding pancreatic cancer, thereby classifying a subject as either positive or negative for pancreatic cancer.

The methods herein enable screening of high-risk subjects, for example, those with a family history of pancreatic cancer, or patients with other risk factors such as chronic pancreatitis, obesity, heavy smoking, and possibly diabetes. The logistic regression model provided herein can incorporate these factors into a classification method.

For subjects that are classified as PDAC-positive, further methods can be provided to clarify PDAC status. Classification as PDAC-positive can be followed by methods including, but not limited to, computed tomography (CT), endoscopic ultrasound (EUS), or endoscopic retrograde cholangiopancreatography (ERCP).

Detection of CA19-9 can be accomplished by contact with the CA19-9 antigen, which is a carbohydrate structure called sialyl-Lewis A (part of the Lewis family of blood group antigens) with the sequence Neu5Acα2,3Galβ1,3 (Fucα1,4) GlcNAc. Sialyl-Lewis A is synthesized by glycosyltransferases that sequentially link the monosaccharide precursors onto both N-linked and O-linked glycans. It is attached to many different proteins, including mucins, carcinoembryonic antigen, and circulating apolipoproteins. In the standard CA19-9 clinical assay, a monoclonal antibody captures and detects the CA19-9 antigen in a sandwich ELISA format, which measures the CA19-9 antigen on many different carrier proteins (Partyka et al., Proteomics 12 (13): 2213-20, 2012).

Detection of TIMP1 (SEQ ID NO:1; UniProtKB: P01033) can be accomplished by contact with a reporter molecule that specifically binds to TIMP1.

```
SEQ ID NO: 1:
                10 20 30 40
MAPFEPLASG ILLLLWLIAP SRACTCVPPH PQTAFCNSDL
                50 60 70 80
VIRAKFVGTP EVNQTILYQR YEIKMTKMYK GFQALGDAAD
                90 100 110 120
IRFVYTPAME SVCGYFHRSH NRSEEFLIAG KLQDGLLHIT
              130 140 150 160
TCSFVAPWNS LSLAQRRGFT KTYTVGCEEC TVFPCLSIPC
              170 180 190 200
KLQSGTHCLW TDQLLQGSEK GFQSRHLACL PREPGLCTWQ
```

Detection of LRG1 (SEQ ID NO:2; UniProtKB: P02750) can be accomplished by contact with a reporter molecule that specifically binds to LRG1.

```
SEQ ID NO: 2:
                10 20 30 40 50
MSSWSRQRPK SPGGIQPHVS RTLFLLLLLA ASAWGVILSP KDCQVERSDH
                60 70 80 90 100
GSSISCQPPA EIPGYLPADT VHLAVEFFNL THLPANLLQG ASKLQELHLS
              110 120 130 140 150
SNGLESLSPE FLRPVPQLRV LDLTRNALTG LPPGLFQASA ILDTLVLKEN
              160 170 180 190 200
QLEVLEVSWL HGLKALGHLD LSGNRLRKLP PGLLANFILL RTLDLGENQL
              210 220 230 240 250
ETLPPDLLRG PLQLERLHLE GNKLQVLGKD LLLPQPDLRY LFLNGNKLAR
              260 270 280 290 300
VAAGAFQGLR QLDMLDLSNN SLASVPEGLW ASLGQPNWDM RDGFDISGNP
                310 320 330 340
WICDQNLSDL YRWLQAQKDK MFSQNDTRCA GPEAVKGQTL LAVAKSQ
```

A combination of at least the three biomarkers CA19-9, TIMP1, and LRG1 can afford a previously unseen, highly reliable PDAC predictive power. When applied to a blind test set composed of plasma samples from 39 resectable PDAC cases and 82 matched healthy controls, the methods described herein yielded an AUC (95% CI) of 0.887 (0.817-0.957) with a sensitivity of 0.667 at 95% specificity in discriminating early-stage PDAC versus healthy controls. The performance of the biomarker panel demonstrated high accuracy detection of early stage pancreatic cancer and a statistically-significant improvement as compared to CA19-9 alone (p=0.008, test set).

With regard to the detection of the biomarkers detailed herein, the disclosure is not limited to the specific biomolecules reported herein. In some embodiments, other biomolecules can be chosen for the detection and analysis of the disclosed biomarkers including, but not limited to, biomolecules based on proteins, antibodies, nucleic acids, aptamers, and synthetic organic compounds. Other molecules may demonstrate advantages in terms of sensitivity, efficiency, speed of assay, cost, safety, or ease of manufacture or storage. In this regard, those of ordinary skill in the art will appreciate that the predictive and diagnostic power of the biomarkers disclosed herein may extend to the analysis of not just the protein form of these biomarkers, but other representations of the biomarkers as well (e.g., nucleic acid). Further, those of ordinary skill in the art will appreciate that the predictive and diagnostic power of the biomarkers disclosed herein can also be used in combination with an analysis of other biomarkers associated with PDAC. In some embodiments, other biomarkers associated with PDAC can be protein-based biomarkers. In some embodiments, other biomarkers associated with PDAC can be non-protein-based biomarkers, such as, for instance, ctDNA.

TIMP1 and LRG1 complement CA19-9 performance in the validation studies that are disclosed herein. Increased gene expression and/or secretion of TIMP1 has been previously observed in PDAC and found to induce tumor cell proliferation. Although elevated circulating TIMP1 levels have been associated with PDAC, increased levels have also been found in other epithelial tumor types. A role for LRG1 has been suggested in promoting angiogenesis through activation of the TGF-β pathway. Besides PDAC, increased LRG1 plasma levels have also been found in other cancer types.

The performance of the three marker panel demonstrated a statistically-significant improvement over CA19-9 alone in distinguishing early-stage PDAC from matched healthy subject or benign pancreatic disease controls. The three marker panel permits assessment of PDAC among subjects at increased risk, namely those with family history, cystic lesions, chronic pancreatitis or subjects who present with adult-onset type II diabetes, as opposed to screening of asymptomatic subjects of average risk.

Disclosed herein is the first proteomics-based study, performed using both human prediagnostic and mouse early-stage PDAC plasma samples, to conduct sequential validation of identified biomarker candidates in multiple independent sets of samples from resectable PDAC patients and matched controls.

In some embodiments, levels of CA19-9, TIMP1, and LRG1 in a biological sample are measured. In some embodiments, CA19-9, TIMP1, and LRG1 are contacted with reporter molecules, and the levels of respective reporter molecules are measured. In some embodiments, three reporter molecules are provided that specifically bind CA19-9, TIMP1, and LRG1, respectively. Use of reporter molecules can provide gains in convenience and sensitivity for the assay.

In some embodiments, CA19-9, TIMP1, and LRG1 are adsorbed onto a surface that is provided in a kit. In some embodiments, reporter molecules bind to surface-adsorbed CA19-9, TIMP1, and LRG1. Adsorption of biomarkers can be nonselective or selective. In some embodiments, the surface comprises a receptor functionality for increasing selectivity towards adsorption of one or more biomarkers.

In some embodiments, CA19-9, TIMP1, and LRG1 are adsorbed onto three surfaces that are selective for one or more of the biomarkers. A reporter molecule or multiple reporter molecules can then bind to surface-adsorbed biomarkers, and the level of reporter molecule(s) associated with a particular surface can allow facile quantification of the particular biomarker present on that surface.

In some embodiments, CA19-9, TIMP1, and LRG1 are adsorbed onto a surface provided in a kit; relay molecules specific for one or more of these biomarkers can bind to surface-adsorbed biomarkers; and receptor molecules specific for one or more relay molecules can bind to relay molecules. Relay molecules can provide specificity for certain biomarkers, and receptor molecules can enable detection.

In some embodiments, three relay molecules are provided that specifically bind CA19-9, TIMP1, and LRG1, respectively. Relay molecules can be designed for specificity towards a biomarker, or can be selected from a pool of candidates due to their binding properties. Relay molecules can be antibodies generated to bind the biomarkers.

In some embodiments, CA19-9, TIMP1, and LRG1 are adsorbed onto three discrete surfaces provided in a kit; relay molecules specific for one or more of these biomarkers can bind to surface-adsorbed biomarkers; and receptor molecules can bind to relay molecules. Analysis of the surfaces can be accomplished in a stepwise or concurrent fashion.

In some embodiments, the reporter molecule is linked to an enzyme, facilitating quantification of the reporter molecule. In some embodiments, quantification can be achieved by catalytic production of a substance with desirable spectroscopic properties.

In some embodiments, the amount of biomarker is determined using spectroscopy. In some embodiments, the spectroscopy is UV/visible spectroscopy. In some embodiments, the amount of biomarker is determined using mass spectrometry.

The quantity of biomarker(s) found in a particular assay can be directly reported to an operator, or alternately it can be stored digitally and readily made available for mathematical processing. A system can be provided for performing mathematical analysis, and can further report classification as PDAC-positive or PDAC-negative to an operator.

In some embodiments, additional assays known to those of ordinary skill in the art can function within the scope of the present disclosure. Examples of other assays include, but are not limited to, assays utilizing mass-spectrometry, immunoaffinity LC-MS/MS, surface plasmon resonance, chromatography, electrochemistry, acoustic waves, immunohistochemistry, and array technologies.

Also provided herein are methods of treatment for subjects who are classified as PDAC-positive. Treatment for PDAC-positive patients can include, but is not limited to, surgery, chemotherapy, radiation therapy, targeted therapy, or a combination thereof.

The foregoing has outlined rather broadly the features and technical benefits of the disclosure in order that the detailed description may be better understood. It should be appreciated by those skilled in the art that the specific embodiments disclosed may be readily utilized as a basis for modifying or designing other structures or processes for carrying out the same purposes of the disclosure. It is to be understood that the present disclosure is not limited to the particular embodiments described, as variations of the particular embodiments may be made and still fall within the scope of the appended claims.

### Definitions

As used herein, the term “pancreatic cancer” means a malignant neoplasm of the pancreas characterized by the abnormal proliferation of cells, the growth of which cells exceeds and is uncoordinated with that of the normal tissues around it.

As used herein, the term “PDAC” refers to pancreatic ductal adenocarcinoma, which is pancreatic cancer that can originate in the ducts of the pancreas.

As used herein, the term “PDAC-positive” refers to classification of a subject as having PDAC.

As used herein, the term “PDAC-negative” refers to classification of a subject as not having PDAC.

As used herein, the term “pancreatitis” refers to an inflammation of the pancreas. Pancreatitis is not generally classified as a cancer, although it may advance to pancreatic cancer.

As used herein, the term “subject” or “patient” as used herein refers to a mammal, preferably a human, for whom a classification as PDAC-positive or PDAC-negative is desired, and for whom further treatment can be provided.

As used herein, a “reference patient” or “reference group” refers to a group of patients or subjects to which a test sample from a patient suspected of having or being susceptible to PDAC may be compared. In some embodiments, such a comparison may be used to determine whether the test subject has PDAC. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual having a healthy pancreas, or normal, non-compromised pancreatic function. A healthy patient or subject has no symptoms of PDAC or other pancreatic disease. In some embodiments, a healthy patient or subject may be used as a reference patient for comparison to diseased or suspected diseased samples for determination of PDAC in a patient or a group of patients.

The term “treatment” or “treating” as used herein refers to the administration of medicine or the performance of medical procedures with respect to a subject, for either prophylaxis (prevention) or to cure or reduce the extent of or likelihood of occurrence or recurrence of the infirmity or malady or condition or event in the instance where the subject or patient is afflicted. As related to the present disclosure, the term may also mean the administration of pharmacological substances or formulations, or the performance of non-pharmacological methods including, but not limited to, radiation therapy and surgery. Pharmacological substances as used herein may include, but are not limited to, chemotherapeutics that are established in the art, such as Gemcitabine (GEMZAR), 5-fluorouracil (5-FU), irinotecan (CAMPTOSAR), oxaliplatin (ELOXATIN), albumin-bound paclitaxel (ABRAXANE), capecitabine (XELODA), cisplatin, paclitaxel (TAXOL), docetaxel (TAXOTERE), and irinotecan liposome (ONIVYDE). Pharmacological substances may include substances used in immunotherapy, such as checkpoint inhibitors. Treatment may include a multiplicity of pharmacological substances, or a multiplicity of treatment methods, including, but not limited to, surgery and chemotherapy.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for PDAC-positive.

As used herein, the term “logistic regression” refers to a regression method in which the assignment of a prediction from the model can have one of several allowed discrete values. For example, the logistic regression models used herein can assign a prediction, for a certain subject, of either PDAC-positive or PDAC-negative.

As used herein, the term “biomarker score” refers to a numerical score for a particular subject that is calculated by inputting the particular biomarker levels for said subject to a statistical method.

As used herein, the term “cutoff point” refers to a mathematical value associated with a specific statistical method that can be used to assign a classification of PDAC-positive of PDAC-negative to a subject, based on said subject's biomarker score.

As used herein, the term “classification” refers to the assignment of a subject as either PDAC-positive or PDAC-negative, based on the result of the biomarker score that is obtained for said subject.

As used herein, the term “PDAC-positive” refers to an indication that a subject is predicted as susceptible to PDAC, based on the results of the outcome of the methods of the disclosure.

As used herein, the term “PDAC-negative” refers to an indication that a subject is predicted as not susceptible to PDAC, based on the results of the outcome of the methods of the disclosure.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or presence of PDAC in subjects of a certain population.

As used herein, the term “true positive rate” refers to the probability that a given subject classified as positive by a certain method is truly positive.

As used herein, the term “false positive rate” refers to the probability that a given subject classified as positive by a certain method is truly negative.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for positive-PDAC and non-positive-PDAC subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, the term “ALCAM” refers to activated leukocyte cell adhesion molecule.

As used herein, the term “CHI3L1” refers to chitinase-3-like-1.

As used herein, the term “COL18A1” refers to collagen type XVIII alpha 1.

As used herein, the term “IGBFP2” refers to insulin-like growth factor binding protein 2.

As used herein, the term “LCN2” refers to lipocalin 2.

As used herein, the term “LRG1” refers to leucine-rich alpha-2-glycoprotein 1.

As used herein, the term “LYZ” refers to lysozyme 2.

As used herein, the term “PARK7,” refers to protein deglycase DJ-1.

As used herein, the term “REG3A” refers to regenerating family member 3 alpha.

As used herein, the term “SLPI” refers to secretory leukocyte protease inhibitor, also known in the art as antileukoproteinase.

As used herein, the term “pro-CTSS” refers to pro-cathepsin S.

As used herein, the term “total-CTSS” refers to total cathepsin S.

As used herein, the term “THBS1” refers to thrombospondin 1.

As used herein, the term “TIMP1” refers to TIMP metallopeptidase inhibitor 1, also known in the art as metalloproteinase inhibitor 1.

As used herein, the term “TNFRSF1A” refers to tumor necrosis factor receptor superfamily member 1A.

As used herein, the term “WFDC2” refers to WAP four-disulfide core domain 2.

As used herein, the term “CA19-9” refers to carbohydrate antigen 19-9, and is also known in the art as cancer antigen 19-9 and sialylated Lewis^{a} antigen.

As used herein, the term “ctDNA” refers to cell-free or circulating tumor DNA. ctDNA is tumor DNA found circulating freely in the blood of a cancer patient. Without being limited by theory, ctDNA is thought to originate from dying tumor cells and can be present in a wide range of cancers but at varying levels and mutant allele fractions. Generally, ctDNA carry unique somatic mutations formed in the originating tumor cell and not found in the host's healthy cells. As such, the ctDNA somatic mutations can act as cancer-specific biomarkers.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, acetylspermidine, diacetylspermine, lysophosphatidylcholine (18:0), lysophosphatidylcholine (20:3) and an indole-derivative.

As used herein, an “indole-derivative” refers to compounds that are derived from indole. Indole is an aromatic heterocyclic organic compound with formula C₈H₇N. It has a bicyclic structure, consisting of a six-membered benzene ring fused to a five-membered nitrogen-containing pyrrole ring. An indole-derivative as described herein may be any derivative of indole. Representative examples include, but are not limited to, tryptophan, indole-3-ethanol, 10,11-Methylenedioxy-20 (S)-CPT, 9-Methyl-20 (S)-CPT, 9-Amino-10,11-methylenedioxy-20 (S)-CPT, 9-Chloro-10,11-methylenedioxy-20 (S)-CPT, 9-Chloro-20 (S)-CPT, 10-Hydroxy-20 (S)-CPT, 9-Amino-20 (S)-CPT, 10-Amino-20 (S)-CPT, 10-Chloro-20 (S)-CPT, 10-Nitro-20 (S)-CPT, 20 (S)-CPT, 9-hydroxy-20 (S)-CPT, (SR)-Indoline-2-carboxylic acid, IAA, IAA-L-Ile, IAA-L-Leu, IBA, ICA-OEt, ICA, Indole-3-acrylic acid, Indole-3-carboxylic acid methyl ester, Indole-3-carboxylic acid, Indole-4-carboxylic acid methyl ester, Boc-L-Ig1-OH.

### Diagnosis, Staging, and Treatment of Pancreatic Cancer.

The most common way to classify pancreatic cancer is to divide it into 4 categories based on whether it can be removed with surgery and where it has spread: resectable, borderline resectable, locally advanced, or metastatic. Resectable pancreatic cancer can be surgically removed. The tumor may be located only in the pancreas or extends beyond it, but it has not grown into important arteries or veins in the area. There is no evidence that the tumor has spread to areas outside of the pancreas. Using standard methods common in the medical industry today, only about 10% to 15% of patients are diagnosed with this stage. Borderline resectable describes a tumor that may be difficult, or not possible, to remove surgically when it is first diagnosed, but if chemotherapy and/or radiation therapy is able to shrink the tumor first, it may be able to be removed later with negative margins. A negative margin means that no visible cancer cells are left behind. Locally advanced pancreatic cancer is still located only in the area around the pancreas, but it cannot be surgically removed because it has grown into nearby arteries or veins or to nearby organs. However, there are no signs that it has spread to any distant parts of the body. Using standard methods common in the medical industry today, approximately 35% to 40% of patients are diagnosed with this stage. Metastatic means the cancer has spread beyond the area of the pancreas and to other organs, such as the liver or distant areas of the abdomen. Using standard methods common in the medical industry today, approximately 45% to 55% of patients are diagnosed with this stage. Alternatively, the TNM Staging System, commonly used for other cancers, may be used (but is not common in pancreatic cancer). This system is based on tumor size (T), spread to lymph nodes (N), and metastasis (M).

Options for treatment of pancreatic cancer include surgery for partial or complete surgical removal of cancerous tissue (for example a Whipple procedure, distal pancreatectomy, or total pancreatectomy), administering one or more chemotherapeutic drugs, and administering therapeutic radiation to the affected tissue (e.g., conventional/standard fraction radiation therapy stereotactic body radiation (SBRT)). Chemotherapeutic drugs approved for treatment of pancreatic cancer include, but are not limited to, capecitabine (Xeloda), erlotinib (Tarceva), fluorouracil (5-FU), gemcitabine (Gemzar), irinotecan (Camptosar), leucovorin (Wellcovorin), nab-paclitaxel (Abraxane), nanoliposomal irinotecan (Onivyde), and oxaliplatin (Eloxatin).

Pancreatic cancer is treated most effectively when diagnosed early, preferably at or before the borderline resectable stage and more preferably at the resectable stage.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of ordinary skill in the art should, in light of the present disclosure, appreciate that many changes can be made in the specific embodiments which are disclosed and still obtain a like or similar result without departing from the spirit and scope of the disclosure.

### Example 1: Mass Spectrometric Methods

Quantitative mass spectrometry (MS) analysis of human plasma samples was done as previously described (Faca et al., PLOS Med. 5 (6): e123, 2008). One pool consisting of pancreatic cases whose blood was collected before onset of symptoms and diagnosis was labeled with heavy 1,2,3-13C-acrylamide isotope while a control pool was labeled with light acrylamide prior to mixing of the pools. Proteins were separated by an automated online 2D-HPLC system controlled by Workstation Class-VP 7.4 (Shimadzu Corporation). Separation consisted of anion exchange chromatography followed by reversed-phase chromatography. Each fraction was lyophilized, in-solution digested, and analyzed by MS using an LTQ-Orbitrap (Thermo) mass spectrometer coupled with a NanoLC-ID (Eksigent).

Acquired LC-MS/MS data were processed by the Computational Proteomics Analysis System (CPAS) pipeline (Rauch et al., J. Proteome Res. 5 (1): 112-21, 2006). X!Tandem, with the custom scoring-plugin Comet, was used as the search engine against the database of human International Protein Index (IPI) version 3.13. Search algorithm parameters were set for trypsin specificity and a maximum of two missed cleavages. Mass tolerance was 1.5 Da for precursor ions and 0.5 Da for fragment ions. Cysteine alkylation with [¹²C] acrylamide (+71.03657) was set as a fixed modification, and [¹³C] acrylamide (+3.01006) and oxidation of methionine (+15.99491) as variable modifications. Identified peptides were further validated through PeptideProphet (Keller et al., Anal. Chem. 74 (20): 5383-92, 2002) and proteins inferred via ProteinProphet (Nesvizhskii et al., Anal. Chem. 75 (17): 4646-58, 2003). Protein identifications were filtered with a 5% error rate based on the ProteinProphet evaluation. Protein quantitative information was extracted with a designated tool Q3 to quantify each pair of peptides containing cysteine residues identified by MS/MS (Faca et al., J. Proteome Res. 5 (8): 2009-18, 2006). Only peptides with a minimum of 0.75 PeptideProphet score, and maximum of 20 ppm fractional delta mass were selected for quantitation. Ratios of [¹³C] acrylamide-labeled to [¹²C] acrylamide-labeled peptides were plotted on a histogram (log 2 scale), and the median of the distribution was centered at zero. All normalized peptide ratios for a specific protein were averaged to compute an overall protein ratio.

The analysis resulted in the identification of 1,732 proteins using ProteinProphet scores of 0.8 or higher, with an error rate less than 5%. Results also included quantification of 395 proteins with at least two quantified peptides used for downstream analysis.

### Example 2: ELISA Methods

For all ELISA experiments, each sample was assayed in duplicate, and the absorbance or chemiluminescence was measured with a SpectraMax M5 microplate reader (Molecular Devices). An internal control sample was run in every plate and each value of the samples was divided by the mean value of the internal control in the same plate to correct inter-plate variability.

### NPC2

Murine monoclonal antibodies (#635 and #675) against recombinant NPC2 (aa 20-151; SEQ ID NO:3; UniProtKB: P61916) were generated and used in a sandwich ELISA.

```
SEQ ID NO: 3:
                10 20 30 40
MRFLAATFLL LALSTAAQAE PVQFKDCGSV DGVIKEVNVS
                50 60 70 80
PCPTQPCQLS KGQSYSVNVI FISNIQSKSS KAVVHGILMG
                90 100 110 120
VPVPFPIPEP DGCKSGINCPI QKDKTYSYL NKLPVKSEYP
              130 140 150
SIKLVVEWQL QDDKNQSLFC WEIPVQIVSH L
```

Ninety-six well polystyrene plates (Corning, Canton, NY, USA) were coated with 1 μg/mL of anti-NPC2 mouse monoclonal antibody (#635) as capture antibody, followed by blocking with Reagent Diluent (R&D Systems). Plasma samples were diluted 1:200 and serial dilution of recombinant protein was applied to develop a standard curve. Biotinylated anti-NPC2 murine monoclonal antibody (#675) at 1:4000 dilutions was used for detection. After washing, each well was incubated with Streptavidin-HRP followed by incubation of color reagents and stop solution (R&D Systems).

### Example 3: Blood Sample Sets

Independent multiple blood sample cohorts were drawn from a pool consisting of PDAC cases (n=187), benign pancreatic disease (n=93), and healthy controls (n=169). All human blood samples were obtained following Institutional Review Board (University of Michigan Comprehensive Cancer Center, Evanston Hospital, University of Utah, University of Texas MD Anderson Cancer Center and International Agency for Research on Cancer) approval and informed consent.

### Initial Discovery Set

For studies using in-depth quantitative MS, a pool of plasma was constituted from 6 pre-diagnostic PDAC cases (sex, male; median age, 66.5 years; range, 62-76 years) and 6 matched controls (sex, male; median age, 67.0 years, range: 61-76 years). These samples were collected from subjects that were subsequently diagnosed with stage IA (N=1), IB (N=2), and IIB (N=3) PDAC an average of 9.3 months (range, 8-12 months) after sample collection as part of the Carotene and Retinol Efficacy Trial and from 6 controls from the same cohort that were matched for age, sex, and smoking history and that were not diagnosed with cancer over a 4-year follow-up period.

### Triage Set

Plasma samples obtained from the University of Michigan Comprehensive Cancer Center under the auspices of the Early Detection Research Network, consisting of 75 PDAC cases, 27 healthy controls, and 19 chronic pancreatitis cases, were used for initial validation and biomarker selection (triage set).

### Validation Sets

An additional set of plasma samples from 73 patients with early-stage PDAC, 60 healthy controls, 60 patients with chronic pancreatitis, and 14 patients with benign pancreatic cysts, were used for biomarker sequential validation and panel development. All chronic pancreatitis samples were collected in an elective setting in the clinic in the absence of an acute flare-up.

Validation set #1, from Evanston Hospital, consisted of stages IB to IIB PDAC cases (n=10), healthy controls (n=10), and chronic pancreatitis cases (n=10); validation set #2, the University of Utah, consisted of early-stage (IA to IIA) PDAC cases (n=42), healthy controls (n=50), and chronic pancreatitis cases (n=50); and validation set #3, the University of Texas MD Anderson Cancer Center, consisted of resectable PDAC cases (n=21) and benign pancreatic cyst cases (n=14).

Demographics for the three validation sets are presented in Table 1.

### Test Set

An additional independent plasma sample set for testing the combined biomarker panel was obtained from the International Agency for Research on Cancer, consisting of 39 early-stage PDAC and 82 healthy controls. Demographics for the test set are presented in Table 2.

TABLE 1
Subject demographics in the three validation sets.

| | | Validation set #1 | | | Validation set #2 | | | Validation set #3 | |
|---|---|---|---|---|---|---|---|---|---|
| | | Pancreatic | Healthy | Chronic | Pancreatic | Healthy | Chronic | Pancreatic | Pancreatic |
| | | cancer | controls | pancreatitis | cancer | controls | pancreatitis | cancer | cyst |
| Total (n) | | 10 | 10 | 10 | 42 | 50 | 50 | 21 | 14 |
| Gender (n) | Male | 4 | 4 | 6 | 26 | 31 | 31 | 11 | 3 |
| | Female | 6 | 6 | 4 | 16 | 19 | 19 | 10 | 11 |
| Age (mean (SD)) | | 74.2 (8.6) | 60.2 (10.4) | 61.6 (13.3) | 64.4 (12.0) | 68.6 (8.3) | 57.9 (14.2) | 64.3 (6.4) | 67.4 (18.0) |
| Stage (n) | IA | — | — | — | 3 | — | — | — | — |
| | IB | 2 | — | — | 9 | — | — | — | — |
| | IIA | 1 | — | — | 30 | — | — | — | — |
| | IIB | 7 | — | — | — | — | — | — | — |
| Clinical | Potentially | — | — | — | — | — | — | 18 | — |
| Stage (n) | Resectable | | | | | | | | |
| | Borderline | — | — | — | — | — | — | 3 | — |
| | Resectable | | | | | | | | |

| | | | | | | | | |
|---|---|---|---|---|---|---|---|---|
| Tumor dimension in cm | — | — | — | 3.2 (1.9) | — | — | — | — |
| (mean (SD))* | | | | | | | | |

*Tumor dimension was available only for validation set #2 and indicates the larger of the two measurements evaluated by computed tomography (CT)/magnetic resonance imaging (MRI)/endoscopic ultrasound (EUS).

TABLE 2
Subject characteristics in the test set.

| | | Pancreatic | Healthy |
|---|---|---|---|
| | | cancer | controls |
| Total (n) | | 39 | 82 |
| Gender (n) | Male | 21 | 43 |
| | Female | 18 | 39 |
| Age (mean (SD)) | | 62.0 (11.0) | 62.8 (10.0) |
| Tobacco | Never | 16 | 41 |
| smoking | | | |
| | Ex-smoker | 12 | 24 |
| | Current smoker | 11 | 17 |
| Alcohol | Never | 23 | 41 |
| drinking | | | |
| | Ex-drinker | 9 | 8 |
| | Current drinker | 7 | 32 |
| | Missing | — | 1 |
| Stage (n) | IA | 6 | — |
| | IB | 10 | — |
| | Resectable | 23 | — |
| | (No TNM data) | | |

### Example 4: Statistical Methods

Raw assay data were log 2-transformed, after imputation of the lowest detected value for each assay, to the values below limit of detection. A one-sided Wilcoxon rank sum test was used to compute p values comparing PDAC cases with healthy controls, chronic pancreatitis cases, and pancreatic cyst cases. The applied test was one-sided as aimed to test the null hypothesis of AUC=0.50 versus the alternative hypothesis AUC>0.50. Receiver operating characteristic (ROC) curve analysis was performed to assess the performance of biomarkers in distinguishing PDAC cases from healthy controls, chronic pancreatitis cases, and pancreatic cyst cases. Owing to the small sample size of each set, validation sets #1, #2, and #3 were merged for model development by standardizing the data such that the mean was 0 and standard deviation was 1 for healthy controls. Because validation set #3 did not include healthy controls, the results were standardized such that the benign pancreatic cyst samples had the same mean and standard deviation as chronic pancreatitis samples. Statistical analyses were performed using MATLAB R2014b and SAS version 9.3. p<0.05 was considered statistically significant in all the analyses.

All possible combinations of seven validated biomarker candidates were explored to select a logistic regression model to discriminate pancreatic cancer from healthy control, chronic pancreatitis and pancreatic cyst based on the Akaike information criterion (AIC). A total of 127 logistic regression models were fitted. Standard errors, confidence intervals, and p values were obtained by 1000 times bootstrap taking into account the variability of the coefficients. The p values for comparing the biomarker panel and CA19-9 alone were calculated by 1000 times bootstrap and refers to the null hypothesis of AUC (panel)=AUC (CA19-9) versus the alternative AUC (panel)>AUC (CA19-9). Likelihood ratio test was also applied to compare the goodness of fit of the biomarker panel to CA19-9 alone. The LeaveMOut cross-validation technique was applied to validate the obtained logistic regression models. Data were split into a training and a test set, which corresponded to ⅔ and ⅓ of the original data, respectively. The models were validated by 1000 repetitions of such a splitting scheme and averaging the obtained 1000 AUCs from the test sets. A modified design covariate matrix was applied to build a logistic regression model with OR rule able to discriminate pancreatic cancer from chronic pancreatitis and benign pancreatic cysts patients: [I(Ca19-9>=a) Ca19-9*I(Ca19-9>=a) I(Ca19-9<a) TIMP1*I(Ca19-9<a) LRG1*I(Ca19-9<a) CA19-9*I(Ca19-9<a)]. All possible values of the CA19-9 threshold “a” were scanned to attain the highest possible AUC by 1000 times bootstrap. The measurements that were not initially selected by the bootstrap were used to generate the predicted scores and evaluate the AUC. The procedure was repeated 1000 times and two-tailed p-values were calculated on the obtained 1000 AUCs. The highest AUC was obtained with “a”=1.6.

To avoid over-fitting in the development in the test set of a logistic regression model which included covariates (represented by recruiting center, gender, age, smoking status, and alcohol drinking) together with the three biomarkers TIMP1, LRG1, CA19-9 a two-step strategy was followed. First a covariate-based score was generated by fitting a logistic regression model which included covariates only, and then the covariate-based score was added to the three-biomarker logistic regression model as a single covariate.

### Example 5: Selection of Biomarker Panel Candidates

The NPC2 assay, as described above, was utilized for this study. At least 17 additional biomarker panel candidates are listed in Table 3.

TABLE 3
Exemplary additional biomarker panel candidates.

| Marker | Vendor | Catalog # | Plasma Dilution |
|---|---|---|---|
| ALCAM | R&D Systems | DY656 | 1:500 |
| CHI3L1 | Quidel | 8020 | 1:4 |
| COL18A1 | R&D Systems | DY1098 | 1:300 |
| IGFBP2 | R&D Systems | DY674 | 1:250 |
| LCN2 | BioPorto | KIT 036 | 1:1500 |
| LRG1 | IBL-America | 27769 | 1:1000 |
| LYZ | ALPCO | K6900 | 1:250 |
| PARK7 | R&D Systems | DY3995 | 1:50 |
| REG3A | DynaBio | PancrePAP | 1:100 |
| SLPI | R&D Systems | DPI00 | 1:20 |
| pro-CTSS | R&D Systems | DY2227 | 1:100 |
| total-CTSS | R&D Systems | DY1183 | 1:50 |
| THBS1 | R&D Systems | DY3074 | 1:1500 |
| TIMP1 | R&D Systems | DY970-05 | 1:500 |
| TNFRSF1A | R&D Systems | DY225-05 | 1:10 |
| WFDC2 | IBL-America | 404-10US | 1:10 |
| CA19-9 | Alpha Diagnostic | 1840 | 1:5 |

### Example 6: Discovery of Biomarker Panel Composition

### Triage

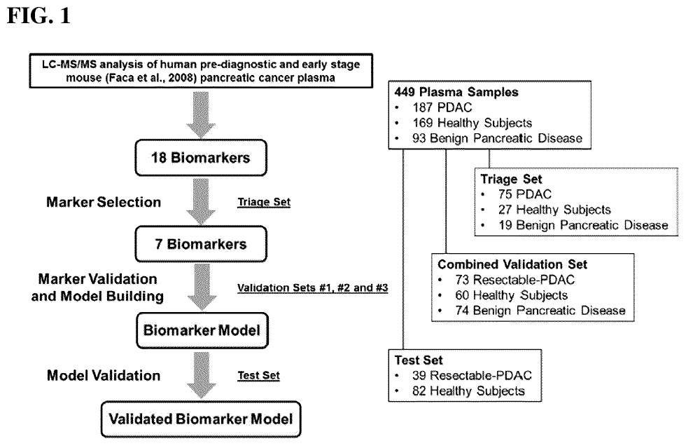

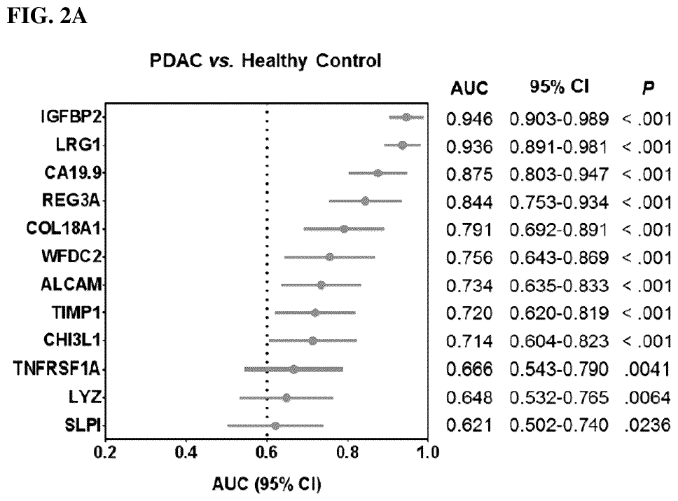

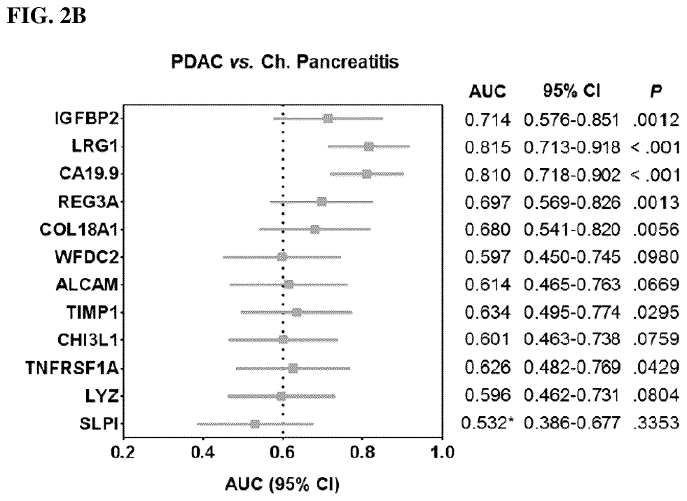

A flow diagram for the study is presented in FIG. 1. Briefly, the pool of 18 biomarker candidates was trimmed by screening against the triage set. The levels of 12 biomarkers were higher to a statistically-significant degree in PDAC compared to healthy controls, each with an area under the curve (AUC)>0.60 and p<0.05 (Wilcoxon rank sum test) (FIG. 2A). The levels of seven of these biomarkers (IGFBP2, LRG1, CA19-9, REG3A, COL18A1, TIMP1, and TNFRSF1A) were also higher to a statistically-significant degree in PDAC cases compared to chronic pancreatitis cases (p<0.05, Wilcoxon rank sum test) with >0.60 of AUC (FIG. 2B). These 7 biomarker candidates were chosen as a triage panel for further evaluation against validation sets #1, #2 and #3.

### Validation

The 7 biomarker candidates in the triage panel were then subjected to analysis with the three validation sets described above. AUC values for all 7 biomarkers selected in the triage set indicate that their plasma levels were consistently elevated in PDAC patients compared with matched controls in validation set #1, #2 and #3 (Tables 4, 5, and 6). The AUCs for these 7 markers, except for IGFBP2 in the comparison of PDAC versus chronic pancreatitis cases in validation set #2, were >0.60 in discriminating PDAC cases from healthy controls as well as chronic pancreatitis cases in both validation set #1 and #2. In addition, 4 biomarkers (CA19-9, TIMP1, LRG1, and IGFBP2) also yielded AUCs>0.60 in plasma samples from PDAC cases compared with benign pancreatic cyst cases in validation set #3 (Table 6).

TABLE 4
Performance of triage panel against validation set #1.

| | Validation set #1 | | | | | | | | | |
|---|---|---|---|---|---|---|---|---|---|---|
| | Pancreatic cancer vs. Healthy control | | | | | Pancreatic cancer vs. Chronic pancreatitis | | | | |
| | | | | Sensitivity | Specificity | | | | Sensitivity | Specificity |
| | | | | at 95% | at 95% | | | | at 95% | at 95% |
| Marker | p | AUC | 95% CI | specificity | sensitivity | p | AUC | 95% CI | specificity | sensitivity |
| LRG1 | <0.001 | 0.940 | 0.841-1.000 | 0.600 | 0.700 | 0.01 | 0.800 | 0.600-1.000 | 0.300 | 0.500 |
| IGFBP2 | 0.001 | 0.890 | 0.722-1.000 | 0.600 | 0.200 | 0.03 | 0.750 | 0.525-0.975 | 0.400 | 0.200 |
| REG3A | 0.006 | 0.840 | 0.650-1.000 | 0.200 | 0.500 | 0.10 | 0.675 | 0.399-0.951 | 0.000 | 0.450 |
| TIMP1 | 0.007 | 0.820 | 0.629-1.000 | 0.500 | 0.400 | 0.08 | 0.690 | 0.447-0.933 | 0.300 | 0.200 |
| CA19-9 | 0.04 | 0.740 | 0.476-1.000 | 0.600 | 0.000 | 0.10 | 0.680 | 0.426-0.934 | 0.400 | 0.100 |
| COL18A1 | 0.08 | 0.690 | 0.437-0.943 | 0.300 | 0.000 | 0.14 | 0.650 | 0.393-0.907 | 0.300 | 0.000 |
| TNFRSF1A | 0.09 | 0.685 | 0.430-0.940 | 0.200 | 0.000 | 0.09 | 0.685 | 0.428-0.942 | 0.100 | 0.000 |

TABLE 5
Performance of triage panel against validation set #2.

| | Validation set #2 | | | | | | | | | |
|---|---|---|---|---|---|---|---|---|---|---|
| | Pancreatic cancer vs. Healthy control | | | | | Pancreatic cancer vs. Chronic pancreatitis | | | | |
| | | | | Sensitivity | Specificity | | | | Sensitivity | Specificity |
| | | | | at 95% | at 95% | | | | at 95% | at 95% |
| Marker | p | AUC | 95% CI | specificity | sensitivity | p | AUC | 95% CI | specificity | sensitivity |
| LRG1 | <0.001 | 0.843 | 0.761-0.925 | 0.381 | 0.260 | 0.002 | 0.675 | 0.563-0.788 | 0.095 | 0.100 |
| IGFBP2 | <0.001 | 0.787 | 0.695-0.878 | 0.357 | 0.440 | 0.08 | 0.587 | 0.469-0.705 | 0.143 | 0.180 |
| REG3A | <0.001 | 0.854 | 0.773-0.934 | 0.476 | 0.342 | <0.001 | 0.694 | 0.586-0.802 | 0.262 | 0.180 |
| TIMP1 | <0.001 | 0.886 | 0.821-0.952 | 0.381 | 0.560 | <0.001 | 0.746 | 0.647-0.845 | 0.190 | 0.360 |
| CA19-9 | <0.001 | 0.885 | 0.808-0.962 | 0.690 | 0.210 | <0.001 | 0.853 | 0.770-0.937 | 0.500 | 0.224 |
| COL18A1 | <0.001 | 0.812 | 0.723-0.901 | 0.333 | 0.260 | 0.001 | 0.686 | 0.575-0.796 | 0.071 | 0.140 |
| TNFRSF1A | <0.001 | 0.745 | 0.646-0.845 | 0.262 | 0.280 | <0.001 | 0.698 | 0.591-0.805 | 0.119 | 0.240 |

TABLE 6
Performance of triage panel against validation set #3.
Validation set #3
Pancreatic cancer vs. Cyst

| | | | | Sensitivity | Specificity |
|---|---|---|---|---|---|
| | | | | at 95% | at 95% |
| Marker | p | AUC | 95% CI | specificity | sensitivity |
| LRG1 | 0.09 | 0.639 | 0.445-0.834 | 0.048 | 0.214 |
| IGFBP2 | 0.07 | 0.653 | 0.460-0.847 | 0.048 | 0.286 |
| REG3A | 0.18 | 0.594 | 0.399-0.788 | 0.190 | 0.071 |
| TIMP1 | 0.002 | 0.798 | 0.637-0.958 | 0.095 | 0.432 |
| CA19-9 | <0.001 | 0.920 | 0.827-1.000 | 0.810 | 0.643 |
| COL18A1 | 0.36 | 0.537 | 0.335-0.740 | 0.143 | 0.071 |
| TNFRSF1A | 0.33 | 0.548 | 0.346-0.749 | 0.000 | 0.071 |

### Panel Construction

To develop a biomarker panel for early-stage PDAC, the results of validation sets #1, #2, and #3 were standardized and combined. In the combined validation set the levels of all 7 biomarkers were higher to a statistically-significant degree (AUC>0.60; p<0.05, Wilcoxon rank-sum test) in PDAC cases than in healthy controls and benign pancreatic disease cases (chronic pancreatitis and benign pancreatic cyst cases combined) (Table 7). Next, a biomarker panel for early-stage PDAC based on a logistic regression model was developed.

The resulting regression model can be:

logit (p)=−1.97+1.7005×logTIMP1+0.93856×logLRG1+0.60639×logCA19.9 where p denotes the probability of being a case in the given sample. This model is a regular logistic regression model that makes use of the logit link function. The binary disease status is playing the role of the response and the markers play the role of the covariates. The algorithm for fitting such regression models is a standard one and is based on an iterative re-weighted procedure which is described in detail in standard textbooks of generalized linear models (McCullogh et al., Generalized Linear and Mixed Models (2008); Wiley Series in Probability and Statistics, John Wiley & Sons, Inc., Hoboken, New Jersey). However, even though this standard approach applies for model fitting it cannot provide inference for the underlying AUC. To provide p-values and confidence intervals that refer to the AUC, a bootstrap scheme was employed in which re-estimation of the coefficients was done within each bootstrap sample (1000 in total) in order to be able to take into account the variability of the estimated coefficients.

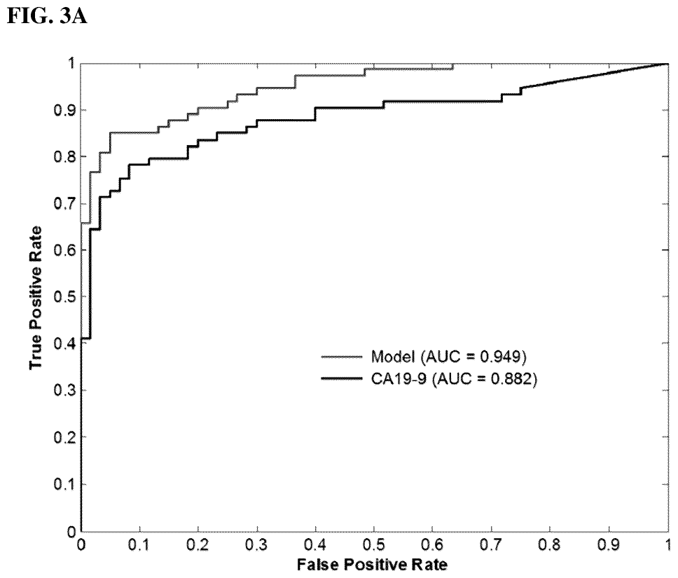

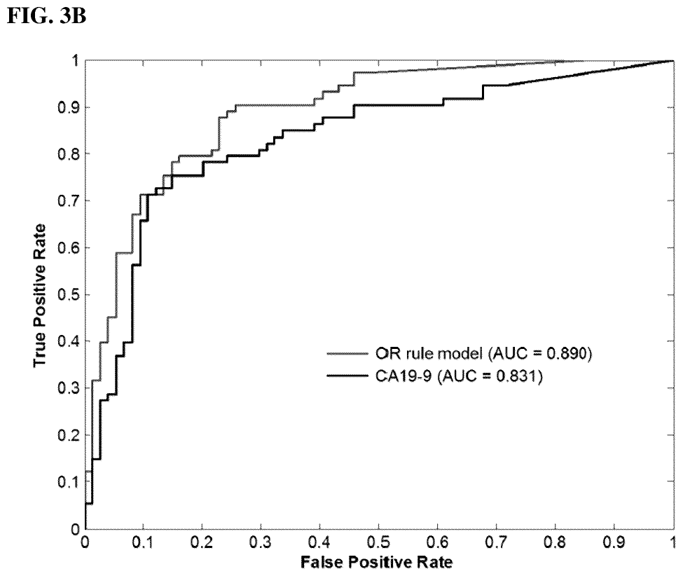

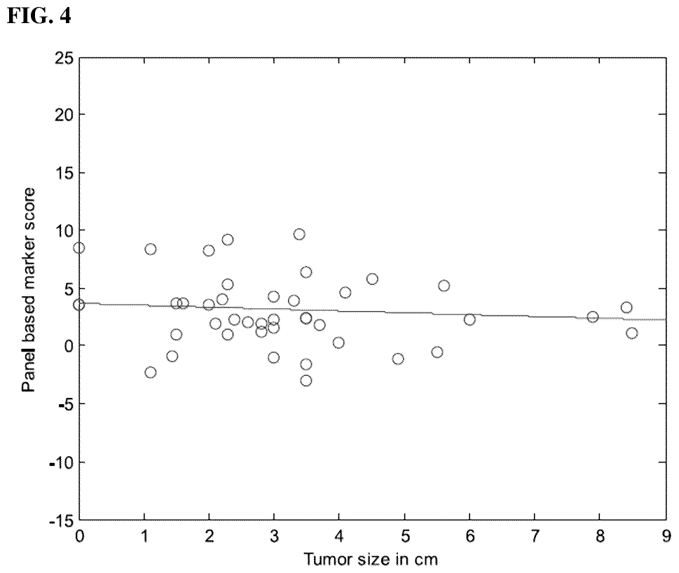

The LeaveMOut cross-validation technique was applied to validate the resulting logistic regression model. In the comparison of PDAC cases with healthy controls, the resulting panel consisted of TIMP1, LRG1, and CA19-9 yielding an AUC (95% CI) of 0.949 (0.917-0.981) and a cross-validation related average AUC of 0.936, which was greater to a statistically-significant degree than the AUC of CA19-9 alone (AUC (95% CI)=0.882 (0.809-0.956); p=0.003, bootstrap; p<0.001, likelihood ratio test; Table 8 and FIG. 3A). The panel yielded a sensitivity of 0.849 and 0.658 at 95% and 99% specificity, respectively, whereas sensitivity at 95% and 99% specificity for CA19-9 alone was 0.726 and 0.411, respectively. A significant improvement over CA19-9 alone in the comparison of PDAC cases with healthy controls was also observed when a model based on the same biomarker combination (TIMP1, LRG1, and CA19-9) was trained in validation set #2 and tested with fixed coefficients in validation set #1 (p=0.04, bootstrap in training set; p=0.02, bootstrap in test set; (Table 9). The results also indicate that in validation set #2, for which tumor size was available, the panel-based biomarker score was not correlated with statistical significance to tumor size. Without being limited by theory, this suggests the ability of the biomarker combination to detect tumors of small dimension (FIG. 4).

A logistic regression model based on the same biomarker combination (TIMP1, LRG1, and CA19-9) was developed to discriminate PDAC from benign pancreatic disease cases (AUC (95% CI)=0.846 (0.781-0.911) and a cross-validation related average AUC=0.830, Table 8). Whether an “OR” rule-based linear regression model, whereby either CA19-9 alone or the combination of all three markers, would enable discrimination between PDAC and benign pancreatic disease cases was also explored. The “OR” rule combination of TIMP1, LRG1, and CA19-9 yielded an AUC (95% CI) of 0.890 (0.802-0.978), which was greater to a statistically-significant degree than that of CA19-9 alone (AUC (95% CI)=0.831 (0.754-0.907); p<0.001 bootstrap; p<0.001, likelihood ratio test; Table 8 and FIG. 3B).

The regression model for discrimination of PDAC from benign pancreatic disease can be:

$$log it ( p ) = - 1.2497 + 0.50306 \times log TIMP 1 + 0.25355 \times log LRG 1 + 0.51564 \times log CA 19.9$$

where log refers to the logarithm with base 2. This was obtained by fitting a regular logistic regression model by employing the logit link function and using the binary disease status as the response and the markers as the covariates. The algorithm for fitting such regression models is a standard one and is based on an iterative re-weighted procedure which is described in detail in standard textbooks of generalized linear models (McCullogh et al., supra). An OR rule was further considered in which a tradeoff between the CA19-9 alone and the three marker panel was considered based on a decision value that was varied through a grid search. Namely a regular logistic regression model was considered for which the design matrix was contributing either only through the CA19-9 or through all three markers. Based on a fine grid of points of the threshold that would determine this contribution, an exemplary AUC was extracted that could be derived after repeatedly fitting all models for every point of the grid.

The panel yielded a sensitivity of 0.452 at 95% specificity, which represents an improvement over a sensitivity of 0.288 at 95% specificity for CA19-9 alone. The “OR” rule combination of TIMP1, LRG1, and CA19-9 resulted in high diagnostic accuracy when applied to the comparison of PDAC patients versus healthy controls yielding an AUC (95% CI) of 0.955 (0.890-1) (p vs. CA19-9: p<0.001 bootstrap; p<0.001, likelihood ratio test; Table 8).

Odds ratios at the Youden index-based optimal cut-off points was estimated. For the model for early-stage PDAC cases versus healthy controls, log (odds ratio) was 4.67 (95% CI=3.29-6.05) at the cut-off point with sensitivity of 0.849 and specificity of 0.950. For the model for early-stage PDAC cases versus benign pancreatic disease cases, log (odds ratio) was 2.98 (95% CI=2.04-3.91) at the cut-off point with sensitivity of 0.863 and specificity of 0.757.

TABLE 7
Performance of biomarkers in combined validation set.

| | | | | | Sensitivity at | Specificity at |
|---|---|---|---|---|---|---|
| | Marker | p | AUC | 95% CI | 95% specificity | 95% sensitivity |
| Pancreatic | CA19-9 | <0.001 | 0.882 | 0.809-0.956 | 0.726 | 0.228 |
| cancer vs. | TIMP1 | <0.001 | 0.880 | 0.805-0.956 | 0.411 | 0.500 |
| Healthy | LRG1 | <0.001 | 0.847 | 0.768-0.926 | 0.425 | 0.250 |
| control | REG3A | <0.001 | 0.819 | 0.735-0.903 | 0.452 | 0.094 |
| | IGFBP2 | <0.001 | 0.800 | 0.715-0.885 | 0.425 | 0.333 |
| | COL18A1 | <0.001 | 0.749 | 0.660-0.837 | 0.329 | 0.233 |
| | TNFRSF1A | <0.001 | 0.692 | 0.597-0.788 | 0.206 | 0.150 |
| Pancreatic | CA19-9 | <0.001 | 0.819 | 0.743-0.895 | 0.288 | 0.243 |
| cancer vs. | TIMP1 | <0.001 | 0.732 | 0.644-0.821 | 0.219 | 0.333 |
| Chronic | LRG1 | <0.001 | 0.682 | 0.592-0.771 | 0.110 | 0.117 |
| pancreatitis | REG3A | <0.001 | 0.656 | 0.563-0.749 | 0.219 | 0.094 |
| | IGFBP2 | 0.005 | 0.624 | 0.529-0.719 | 0.274 | 0.167 |
| | COL18A1 | 0.005 | 0.628 | 0.531-0.725 | 0.082 | 0.133 |
| | TNFRSF1A | 0.002 | 0.643 | 0.548-0.738 | 0.096 | 0.100 |
| Pancreatic | CA19-9 | <0.001 | 0.831 | 0.754-0.907 | 0.288 | 0.259 |
| cancer vs. | TIMP1 | <0.001 | 0.742 | 0.657-0.828 | 0.206 | 0.324 |
| Benign | LRG1 | <0.001 | 0.679 | 0.580-0.772 | 0.110 | 0.135 |
| pancreatic | REG3A | <0.001 | 0.651 | 0.560-0.743 | 0.192 | 0.090 |
| disease* | IGFBP2 | 0.002 | 0.632 | 0.542-0.722 | 0.219 | 0.189 |
| | COL18A1 | 0.004 | 0.627 | 0.534-0.719 | 0.082 | 0.149 |
| | TNFRSF1A | 0.001 | 0.643 | 0.551-0.736 | 0.082 | 0.122 |

*Benign pancreatic disease (chronic pancreatitis cases and benign pancreatic cyst cases).

TABLE 8
Performance of biomarker panel in the combined validation set.

| | | | Sensitivity | Sensitivity | Specificity | Specificity | CV- | p (vs. CA19-9) | |
|---|---|---|---|---|---|---|---|---|---|
| | | | at 95% | at 99% | at 95% | at 99% | AUC | | Likelihood |
| Model | AUC | 95% CI | specificity | specificity | sensitivity | sensitivity | (SD) | Bootstrap | ratio test |
| Pancreatic cancer vs. | | | | | | | | | |
| Healthy Control | | | | | | | | | |
| TIMP1 + LRG1 + | 0.949 | 0.917-0.981 | 0.849 | 0.658 | 0.633 | 0.367 | 0.936 | 0.003 | <0.001 |
| CA19-9 (linear) | | | | | | | (0.030) | | |
| TIMP1 + LRG1 + | 0.955 | 0.890-1 | 0.849 | 0.575 | 0.667 | 0.389 | 0.968 | <0.001 | <0.001 |
| CA19-9 (“OR” rule) | | | | | | | (0.022) | | |
| Pancreatic cancer vs. | | | | | | | | | |
| Benign pancreatic disease* | | | | | | | | | |
| TIMP1 + LRG1 + | 0.846 | 0.781-0.911 | 0.356 | 0.110 | 0.351 | 0.108 | 0.830 | 0.18 | 0.02 |
| CA19-9 (linear) | | | | | | | (0.049) | | |
| TIMP1 + LRG1 + | 0.890 | 0.802-0.978 | 0.452 | 0.123 | 0.541 | 0.282 | 0.887 | <0.001 | <0.001 |
| CA19-9 (“OR” rule) | | | | | | | (0.041) | | |

*Benign pancreatic disease (chronic pancreatitis cases and benign pancreatic cyst cases).
CV-AUC: cross-validation related average AUC.

TABLE 9
Performance of biomarker panel in the validation set #1 and #2.

| | | | | Sensitivity | Sensitivity | Specificity | Specificity | | p (vs. CA19-9)* | |
|---|---|---|---|---|---|---|---|---|---|---|
| | | | | at 95% | at 99% | at 95% | at 99% | CA19-9 | | Likelihood |
| | | AUC | 95% CI | specificity | specificity | sensitivity | sensitivity | AUC | Bootstrap | ratio test |
| Training | Validation set #2 | 0.937 | 0.892-0.983 | 0.762 | 0.619 | 0.680 | 0.460 | 0.885 | 0.04 | <0.001 |
| Testing | Validation set #1 | 0.930 | 0.826-1 | 0.800 | 0.800 | 0.600 | 0.600 | 0.740 | −0.02 | — |

*Statistical tests were one-sided.

### Example 7: Evaluation of Biomarker Panel

Further blinded validation of the panel of three biomarkers TIMP1, LRG1, and CA19-9 was performed using the test set. The levels of all 3 biomarkers were significantly higher in PDAC cases than in healthy controls with AUC (95% CI) of 0.821 (0.736-0.906) for CA19-9, 0.730 (0.626-0.834) for TIMP1, and 0.832 (0.755-0.909) for LRG1 (Table 10). A linear combination of the three markers yielded an AUC (95% CI) of 0.903 (0.838-0.967), which was greater to a statistically-significant degree than the AUC of CA19-9 alone (p=0.001, bootstrap; p<0.001, likelihood ratio test; Table 11). Moreover, the linear combination of TIMP1, LRG1, CA19-9 and covariates (represented by recruiting center, gender, age, smoking status, and alcohol consumption) yielded an AUC (95% CI) of 0.929 (0.878-0.980), which represents a statistically-significant improvement over CA19-9 and covariates combination alone (AUC (95% CI)=0.848 (0.778-0.920); p=0.01, bootstrap; p<0.001, likelihood ratio test; Table 11). Inclusion of covariates resulted in a statistically-significant improvement in performance as compared to the three biomarker panel alone (p=0.03, bootstrap; p=0.004, likelihood ratio test; Table 11).

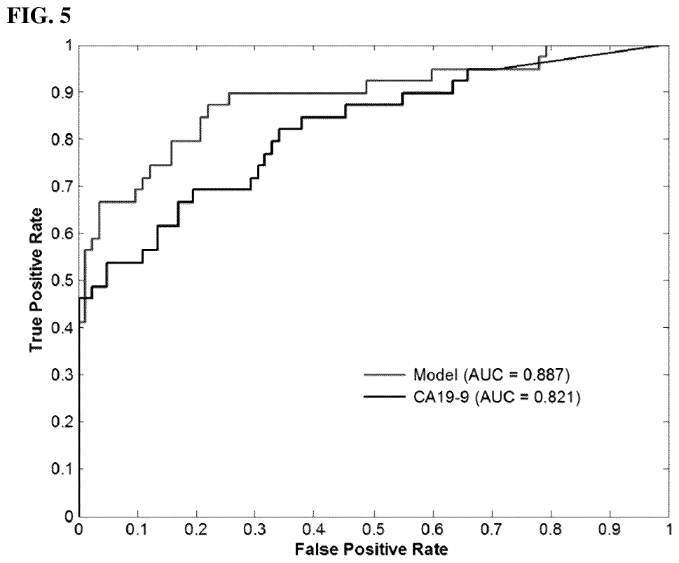

Of note, the logistic regression model of CA19-9, TIMP1 and LRG1 with fixed coefficients which was developed in the combined validation sets for PDAC versus healthy controls yielded an AUC of 0.887, also with statistically-significant improved performance compared to CA19-9 alone (p=0.008, likelihood ratio test; Table 10 and FIG. 5). The model yielded a sensitivity of 0.667 and 0.410 at 95% and 99% specificity, respectively, whereas sensitivity at 95% and 99% specificity for CA19-9 alone was 0.538 and 0.462, respectively. Log-transformed odds ratios at the Youden index-based optimal cut-off points was 3.19 (95% CI=2.11-4.26) at the cut-off point with sensitivity of 0.872 and specificity of 0.780.

### Example 8: Specificity and Sensitivity in the Range of Regression Model Diagnostic Scores

It will be appreciated by those of ordinary skill in the art that different methods or assays of biomarker detection, quantitation, and analysis, which can include using different reagents, will produce different results which may require modification of the regression model. In particular, different assays can produce results expressed, for example, in different units. Further, duplicate reactions in duplicate assays of the same samples can also produce different raw results. However, it is the combined detection, quantitation, and analysis of at least the three biomarkers TIMP1, LRG1, and CA19-9 that, when incorporated into a regression model as disclosed herein, produce a definitive diagnosis of PDAC.

A range in the results reported for each particular assay used to detect, quantify, and analyze the three biomarkers will have a range in the resulting PDAC-predictive score that depends, in part, on the degree of sensitivity or specificity (Table 12; where the preferred cutoff based on the Youden Index is 0.8805 with a specificity of 0.95 and sensitivity of 0.8493). The regression model used to generate the PDAC-predictive score can depend on the specific assays utilized to test the markers. As understood by those of skill in the art, different assays can target different epitopes of the three biomarkers or have different affinities and sensitivities. As such, the regression model algorithm used to generate the PDAC-predictive score can be modified to take these assay variations into consideration.

### Example 9: Assaying Samples and PDAC-Patient Diagnosis

In one example, a patient being screened for PDAC-based on the three-biomarker panel disclosed herein—has a blood sample drawn (or other fluid or tissue biopsy) and assayed by ELISA (or other assay) to quantitate the levels of TIMP1, LRG1, and CA19-9 in the patient. Normalized values for at least these biomarkers that take into account the specific assay used (e.g., ELISA; Table 3) could be, for example, TIMP1=0.6528 ng/ml; LRG1=2.0498 ng/ml; and CA19-9=1.8160 U/mL. Raw assay data are then log 2-transformed, computing the mean and standard deviation for the healthy samples in each cohort. The data is then standardized so that healthy samples have a mean of 0 and a standard deviation of 1: where (Readⱼ−mean_{healthy})/(std_{healthy}), where j is the jth sample.

When analyzed using the following regression model:

logit (p)=−1.97+1.7005×logTIMP1+0.93856×logLRG1+0.60639×logCA19.9 the above patient would have a combined score of 2.1653. In view of the preferred cutoff for consideration of both specificity and sensitivity (Table 12), a patient with such a combined score would have PDAC with near certainty and consequently be directed for follow-up testing and treatment for PDAC using other modalities discussed herein and known to those of skill in the art. Using the regression model described herein, the more positive the combined PDAC-predictive score, the more certainty the patient has PDAC. Conversely, the more negative the combined PDAC-predictive score, the more certainty the patient does not have PDAC.

By contrast, in another example, normalized values for biomarkers TIMP1, LRG1, and CA19-9 that take into account the specific assay used could be, for example, TIMP1=−2.0370 ng/ml; LRG1=−1.5792 ng/mL; and CA19-9=1.0712 U/mL. When analyzed using the same regression model as above, such a patient would have a combined score of −6.2666. In view of the preferred cutoff for consideration of both specificity and sensitivity (Table 12), a patient with such a combined score would, with near certainty, not have PDAC and, therefore, would or would not need to be followed for additional testing based on the strength of any other clinical conditions.

TABLE 10
Performance of biomarker model in the test set.

| | | | | Sensitivity | Sensitivity | Specificity | Specificity | |
|---|---|---|---|---|---|---|---|---|
| | | | | at 95% | at 99% | at 95% | at 99% | p (vs. |
| Marker | AUC | p | 95% CI | specificity | specificity | sensitivity | sensitivity | CA19-9)* |
| CA19-9 | 0.821 | <0.001 | 0.736-0.906 | 0.538 | 0.462 | 0.286 | 0.067 | — |
| TIMP1 | 0.730 | <0.001 | 0.626-0.834 | 0.359 | 0.333 | 0.085 | 0.000 | — |
| LRG1 | 0.832 | <0.001 | 0.755-0.909 | 0.462 | 0.179 | 0.366 | 0.220 | — |
| Model (fixed coefficients): | 0.887 | <0.001 | 0.817-0.957 | 0.667 | 0.410 | 0.220 | 0.207 | 0.008 |
| TIMP1 + LRG1 + CA19-9 | | | | | | | | |

TABLE 11
Performance of biomarker panel in the test set.

| | | | | Sensitivity | Sensitivity | Specificity | Specificity | p (vs. CA19-9) | |
|---|---|---|---|---|---|---|---|---|---|
| | | | | at 95% | at 99% | at 95% | at 99% | | Likelihood |
| Marker | AUC | P* | 95% CI | specificity | specificity | sensitivity | sensitivity | Bootstrap | ratio test |
| CA19-9 | 0.821 | <0.001 | 0.736-0.906 | 0.538 | 0.462 | 0.286 | 0.067 | — | — |
| CA19-9 + Covariates^{†} | 0.848 | <0.001 | 0.778-0.920 | 0.513 | 0.308 | 0.407 | 0.321 | 0.02 | 0.007 |
| Panel: TIMP1 + LRG1 + CA19-9 | 0.903 | <0.001 | 0.838-0.967 | 0.692 | 0.282 | 0.247 | 0.247 | 0.001 | <0.001 |
| Panel + Covariates^{†} | 0.929 | <0.001 | 0.878-0.980 | 0.821 | 0.077 | 0.481 | 0.407 | 0.01^{‡} | <0.001^{‡} |
| | | | | | | | | 0.03^{§} | 0.004^{§} |

*p values were calculated using a one-sided Wilcoxon rank sum test.
^{†}Covariates: recruiting center, gender, age (continuous), smoking status (current, ex-, never smoker), and alcohol drinking (current, ex-, never drinker). One healthy control subject with missing alcohol consumption information was not included in this analysis.
^{‡}p versus CA19-9 + Covariates
^{§}p versus Panel

TABLE 12
Sensitivity and specificity at different cutoffs of the biomarker
panel-based (TIMP1, LRG1, and CA19-9) scores
in the combination validation set.

| Cutoff | Specificity | Sensitivity |
|---|---|---|
| −15.0 | 0.000 | 1.000 |
| −14.5 | 0.000 | 1.000 |
| −14.0 | 0.000 | 1.000 |
| −13.5 | 0.000 | 1.000 |
| −13.0 | 0.000 | 1.000 |
| −12.5 | 0.000 | 1.000 |
| −12.0 | 0.000 | 1.000 |
| −11.5 | 0.017 | 1.000 |
| −11.0 | 0.017 | 1.000 |
| −10.5 | 0.017 | 1.000 |
| −10.0 | 0.017 | 1.000 |
| −9.5 | 0.017 | 1.000 |
| −9.0 | 0.017 | 1.000 |
| −8.5 | 0.017 | 1.000 |
| −8.0 | 0.017 | 1.000 |
| −7.5 | 0.017 | 1.000 |
| −7.0 | 0.033 | 1.000 |
| −6.5 | 0.050 | 1.000 |
| −6.0 | 0.067 | 1.000 |
| −5.5 | 0.083 | 1.000 |
| −5.0 | 0.100 | 1.000 |
| −4.5 | 0.117 | 1.000 |
| −4.0 | 0.183 | 1.000 |
| −3.5 | 0.267 | 1.000 |
| −3.0 | 0.367 | 0.986 |
| −2.5 | 0.467 | 0.986 |
| −2.0 | 0.567 | 0.973 |
| −1.5 | 0.633 | 0.945 |
| −1.0 | 0.733 | 0.932 |
| −0.5 | 0.800 | 0.890 |
| 0.0 | 0.850 | 0.877 |
| 0.5 | 0.883 | 0.849 |
| 1.0 | 0.950 | 0.836 |
| 1.5 | 0.967 | 0.767 |
| 2.0 | 1.000 | 0.658 |
| 2.5 | 1.000 | 0.521 |
| 3.0 | 1.000 | 0.507 |
| 3.5 | 1.000 | 0.411 |
| 4.0 | 1.000 | 0.274 |
| 4.5 | 1.000 | 0.247 |
| 5.0 | 1.000 | 0.205 |
| 5.5 | 1.000 | 0.178 |
| 6.0 | 1.000 | 0.164 |
| 6.5 | 1.000 | 0.151 |
| 7.0 | 1.000 | 0.151 |
| 7.5 | 1.000 | 0.151 |
| 8.0 | 1.000 | 0.137 |
| 8.5 | 1.000 | 0.096 |
| 9.0 | 1.000 | 0.096 |
| 9.5 | 1.000 | 0.055 |
| 10.0 | 1.000 | 0.041 |
| 10.5 | 1.000 | 0.041 |
| 11.0 | 1.000 | 0.041 |
| 11.5 | 1.000 | 0.027 |
| 12.0 | 1.000 | 0.027 |
| 12.5 | 1.000 | 0.027 |
| 13.0 | 1.000 | 0.027 |
| 13.5 | 1.000 | 0.027 |
| 14.0 | 1.000 | 0.027 |
| 14.5 | 1.000 | 0.027 |
| 15.0 | 1.000 | 0.014 |
| 15.5 | 1.000 | 0.014 |
| 16.0 | 1.000 | 0.014 |
| 16.5 | 1.000 | 0.014 |
| 17.0 | 1.000 | 0.014 |
| 17.5 | 1.000 | 0.014 |
| 18.0 | 1.000 | 0.014 |
| 18.5 | 1.000 | 0.014 |
| 19.0 | 1.000 | 0.014 |
| 19.5 | 1.000 | 0.014 |
| 20.0 | 1.000 | 0.014 |
| 20.5 | 1.000 | 0.014 |
| 21.0 | 1.000 | 0.000 |
| 21.5 | 1.000 | 0.000 |
| 22.0 | 1.000 | 0.000 |
| 22.5 | 1.000 | 0.000 |
| 23.0 | 1.000 | 0.000 |
| 23.5 | 1.000 | 0.000 |
| 24.0 | 1.000 | 0.000 |
| 24.5 | 1.000 | 0.000 |
| 25.0 | 1.000 | 0.000 |

### Example 10: A Panel Combining Plasma Metabolite and Protein Markers for the Detection of Early Stage Pancreatic Cancer

Using an untargeted metabolomics approach, a plasma-derived metabolite biomarker panel was developed for resectable pancreatic ductal adenocarcinoma (PDAC). A multi-assay metabolomics approach using liquid chromatography/mass spectrometry was applied on plasmas collected from 20 (10 early and 10 late stage) PDAC cases and 20 matched controls (10 healthy subjects; 10 subjects with chronic pancreatitis) to identify candidate metabolite markers for PDAC; candidate markers were narrowed based on a second ‘confirmatory’ cohort consisting of 9 PDACs and 50 subjects with benign pancreatic disease (BPD). Blinded validation was performed in an independent cohort consisting of 39 resectable PDAC cases and 82 matched controls. Five metabolites, including acetylspermidine, diacetylspermine, lysophosphatidylcholine (18:0), lysophosphatidylcholine (20:3) and an indole-derivative, were identified in discovery and ‘confirmatory’ cohorts as candidate biomarkers markers for PDAC. A metabolite panel was developed based on logistic regression models and evaluated for its ability to distinguish PDAC from healthy controls in the combined discovery and ‘confirmatory’ cohort. The resulting panel yielded an area under the curve (AUC) of 0.90 (95% C.I.: 0.818-0.989). Blinded validation of the metabolite panel yielded an AUC of 0.89 (95% C.I.: 0.828-0.956) in the independent validation cohort. Importantly evaluation of the metabolite markers in combination with our previously identified protein markers (CA19-9, TIMP1 and LRG1) yielded an AUC of 0.92 in the validation cohort, which was statistically significantly greater than the protein panel alone (AUC=0.86; p-value: 0.024), highlighting the complementary nature of the metabolite panel when combined with a three-protein marker panel.

Pancreatic ductal adenocarcinoma (PDAC) is the third leading cause of cancer-related mortality in both men and women in the United States, with an overall 5-year survival rate of only ˜8%. Unfortunately, diagnosis of PDAC at an early stage is uncommon and usually incidental in the majority of patients (˜85%) presenting with locally advanced or metastatic disease.

Currently, no clinical marker(s) exist that display desired performance characteristics for early stage PDAC in asymptomatic individuals. The current use of CA19-9 as a screen biomarker is limited by its variable accuracy, reduced performance in pre-diagnostic stages of the disease, and its inability to be detected in ˜10% of subjects with fucosyltransferase deficiency. Consequently, there is a critical need for additional markers that display collectively higher sensitivity and specificity for reliable detection of low volume PDAC in asymptomatic individuals. In this context, blood-based biomarker(s) are ideal and represent a relatively non-invasive and cost-effective method for detecting disease at early stages.

Recently, development and sequential validation of a protein-based biomarker panel for detecting early-stage PDAC, capable of complementing CA19-9 was performed. Although classification performance improved relative to CA19-9 alone, room for improvement remained. Thus, there is a need to test the relative contribution of different types of biomarkers, such as metabolites, to enable the development of an optimal biomarker combination model for this challenging application.

In the present study, an untargeted metabolomics approach was applied to develop a plasma-derived metabolite biomarker panel for PDAC. The fixed biomarker panel was subsequently blindly validated in an independent test cohort consisting of 39 resectable PDAC cases and 82 matched healthy controls in addition to being compared against a previously identified protein panel. The performance of the metabolite panel was additionally tested to distinguish PDAC cases from subjects diagnosed with benign pancreatic cysts.

### Study Population

All human blood samples were obtained following Institutional Review Board approval and informed consent. For initial metabolite discovery studies, plasma samples from 20 patients with PDAC, including 10 early-stage and 10 late stage PDAC, 10 healthy controls, and 10 patients with chronic pancreatitis were obtained from the Evanston Hospital (discovery set). All chronic pancreatitis samples were collected in an elective setting in the clinic in the absence of an acute flare-up. Plasma samples obtained from the Indiana University School of Medicine, consisting of 50 patients with low dysplastic grade pancreatic cyst and 9 patients with invasive IPMN (5 early-stage and 4 late-stage adenocarcinoma) were used for biomarker sequential selection and initial validation (confirmation set). All patients underwent surgical resection of their cystic lesion, and plasma samples were collected prior to surgery. Dysplastic grade was histopathology confirmed after surgical resection and determined according to WHO criteria. An additional independent plasma sample set for testing the combined biomarker panel was obtained from the International Agency for Research on Cancer, consisting of 39 early-stage PDAC and 82 healthy controls (Test Set #1). A second sample set from the Indiana University School of Medicine, consisting of 102 patients with low dysplastic grade pancreatic cyst, 12 patients with resectable invasive IPMN, and resectable 8 PDAC patients with IPMN was applied as a Test Set #2. Study flow diagram and clinical characteristics of the patients in the validation sets and test set are presented in FIG. 6 and Tables 13 and 14.

TABLE 13
Subject Characteristics in the Discovery Sets.

| | | Discovery Cohort (Set #1) | | |
|---|---|---|---|---|
| | | Pancreatic | Healthy | Chronic |
| | | cancer | controls | pancreatitis |
| Total (n) | | 20 | 10 | 10 |
| Gender (n) | Male | 10 | 4 | 6 |
| | Female | 10 | 6 | 4 |
| Age (mean (SD)) | | 70.4 (10.0) | 60.2 (10.4) | 61.6 (13.3) |
| Stage (n) | IB | 2 | — | — |
| | IIA | 1 | — | — |
| | IIB | 7 | — | — |
| | IV | 10 | — | — |

| | | ‘Confirmatory’ Cohort (Set #2) | |
|---|---|---|---|
| | | Pancreatic | Low grade |
| | | cancer | pancreatic cyst |
| Total (n) | | 9 | 50 |
| Gender (n) | Male | 3 | 18 |
| | Female | 6 | 32 |
| Age (mean (SD)) | | 73.1 (8.1) | 62.5 (17.5) |
| Tobacco | Never | 5 | 22 |
| smoking | | | |
| | Ex-smoker | 3 | 16 |
| | Current smoker | 1 | 11 |
| | Missing | — | 1 |
| Type 2 diabetes | Yes | 4 | 34 |
| | No | 5 | 16 |
| Alcohol | Never | 6 | 31 |
| drinking | | | |
| | Ex-drinker | — | 8 |
| | Current drinker | 3 | 9 |
| | Missing | — | 2 |
| Cystic lesion | IPMN | 9 | 34 |
| | MCN | — | 11 |
| Stage (n) | SCN | — | 5 |
| | IA | 1 | — |
| | IIA | 2 | — |
| | IIB | 2 | — |
| | IV | 4 | — |

TABLE 14
Subject Characteristics in the Test Sets.

| | | Test Set #1 | |
|---|---|---|---|
| | | Pancreatic | Healthy |
| | | cancer | controls |
| Total (n) | | 39 | 82 |
| Gender (n) | Male | 21 | 43 |
| | Female | 18 | 39 |
| Age (mean (SD)) | | 62.0 (11.0) | 62.8 (10.0) |
| Tobacco | Never | 16 | 41 |
| smoking | | | |
| | Ex-smoker | 12 | 24 |
| | Current smoker | 11 | 17 |
| Alcohol | Never | 23 | 41 |
| drinking | | | |
| | Ex-drinker | 9 | 8 |
| | Current drinker | 7 | 32 |
| | Missing | — | 1 |
| Stage (n) | IA | 6 | — |
| | IB | 10 | — |
| | Resectable | 23 | — |
| | (No TNM data) | | |
| | | Test Set #2 | |
| | | Pancreatic | Low grade |
| | | cancer | pancreatic cyst |
| Total (n) | | 20 | 102 |
| Gender (n) | Male | 12 | 43 |
| | Female | 8 | 59 |
| Age (mean (SD)) | | 69.6 (11.4) | 64.5 (12.6) |
| Tobacco | Never | 9 | 51 |
| smoking | | | |
| | Ex-smoker | 7 | 22 |
| | Current smoker | 4 | 29 |
| Type 2 diabetes | Yes | 7 | 20 |
| | No | 13 | 78 |
| | Missing | — | 4 |
| Alcohol | Never | 8 | 66 |
| drinking | | | |
| | Ex-drinker | 2 | 9 |
| | Current drinker | 9 | 27 |
| | Missing | 1 | — |
| Cystic lesion | IPMN | 12 | 92 |
| | MCN | — | 7 |
| | SCN | — | 3 |
| | Adenocarcinoma | 8 | — |
| | with IPMN | | |
| Stage (n) | IA | 6 | — |
| | IIA | 4 | — |
| | IIB | 10 | — |

### Cell Line Metabolomic Experiments

PDAC cell lines (CFPAC, MiaPaCa, SU8686, BxPC3, CAPAN2, PANC03.27 and SW 1990) were grown in RPMI-1640 with 10% FBS. The identity of each cell line was confirmed by DNA fingerprinting via short tandem repeats at the time of mRNA and total protein lysate preparation using the PowerPlex 1.2 kit (Promega). Fingerprinting results were compared with reference fingerprints maintained by the primary source of the cell line. Cells were seeded in 6-cm dishes (Thermo Scientific) to reach a 70% (50-80%) confluency, 24 hours post initial seeding. Post 24 hours, cell lysates were washed 2× with pre-chilled 0.9% NaCl followed by addition of 1 mL of pre-chilled extraction buffer (3:1 isopropanol:ultrapure water) to quench and remove cell media. Cells were then scraped using a 25-cm Cell Scraper (Sarstedt) in extraction solvent and transferred to a 1.5-mL Eppendorf tube. After vortexing briefly, the extracted cell lysates were centrifuged at 4° C. for 10 min at 2,000×g. Thereafter, 1 mL of the supernatant containing the extracted metabolites were transferred to 1.5-mL Eppendorf tubes and stored in −20° C. until needed for metabolomic analysis.

### Exometabolome Experiments

Cells were grown in 1 ml of RPMI 1640+10% FBS in 12-well dishes (Costar) to reach a 70% (50-80%) confluency, 24 hours post initial seeding. On the day of the experiment, the cells were washed 2 times with 500 μL serum-free RPMI (Fisher Scientific) containing 5 mM glucose and 0.5 mM glutamine. Serum-free RPMI (300 μL) containing 5 mM glucose and 0.5 mM glutamine was then added to each well and the cells were incubated. After a predetermined incubation time (1, 2, 4, and 6 hours) 250 μL of the conditioned media was collected. For baseline (TO), 250 μL of media was collected directly after the addition of 300 μL. All time points were performed in triplicates or quadruplicates. Blank samples containing media only were included and collected at T0 and T6. The 6-hour samples were used to count cell numbers for data normalization. Once all the media samples were collected, the tubes were centrifuged at 2000×g for 10 min to remove residual debris and the supernatants transferred to 1.5-mL Eppendorf tubes and stored at −80° C. until used for metabolomics analysis.

### Primary Metabolites and Biogenic Amines

Plasma metabolites were extracted from pre-aliquoted EDTA plasma (10 μL) with 30 μL of LCMS grade methanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 10 μL of 100 mM ammonium formate, pH 3. For Hydrophilic Interaction Liquid Chromatography (HILIC) analysis, the samples were diluted with 60 μL LCMS grade acetonitrile (ThermoFisher), whereas samples for C18 analysis were diluted with 60 μL water (GenPure ultrapure water system, ThermoFisher). Each sample solution was transferred to 384-well microplate (Eppendorf) for LCMS analysis.

For cell lysates, 100 μL (3:1 isopropanol:ultrapure water) was aliquoted into two 300 μL, 96-well plates (Eppendorf) and evaporated to dryness under vacuum. The samples were then reconstituted as follows: for the HILIC assays, the dried samples were dissolved in 65 μL of ACN (Fisher Scientific): 100 mM Ammonium Formate pH 3 (9:1), whereas for the reverse phase C18 assays, the dried samples were dissolved in 65 μL of H₂O: 100 mM Ammonium Formate pH 3 (9:1). The samples were then spun down to remove any insoluble materials and transferred to a 384-well plate for high throughput analysis using LCMS.

Frozen media samples were thawed on ice and 30 μl transferred to a 96-well microplate (Eppendorf) containing 30 μL of 100 mM ammonium formate, pH 3.0. The microplates were heat sealed, vortexed for 5 min at 750 rpm, and centrifuged at 2000×g for 10 minutes at room temperature. For Hydrophilic Interaction Liquid Chromatography (HILIC) analysis, 25 μL of sample was transferred to a new 96-well microplate containing 75 μL acetonitrile, whereas samples for C18 analysis were transferred to a new 96-well microplate containing 75 μL water (GenPure ultrapure water system, ThermoFisher). Each sample solution was transferred to 384-well microplate (Eppendorf) for LCMS analysis.

For each batch, samples were randomized and matrix-matched reference quality controls and batch-specific pooled quality controls were included.

### Complex Lipids

Pre-aliquoted EDTA plasma samples (10 μL) were extracted with 30 μL of LCMS grade 2-propanol (ThermoFisher) in a 96-well microplate (Eppendorf). Plates were heat-sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 90 μL of 1:3:2 100 mM ammonium formate, pH 3 (Fischer Scientific): acetonitrile: 2-propanol and transferred to a 384-well microplate (Eppendorf) for lipids analysis using LCMS.

For cell lysates, in a 300 μL, 96-well plate, 10 μL supernatant (3:1 isopropanol:ultrapure water) of the extracted cell lysate metabolites was diluted with 90 μL of 1:3:2 100 mM ammonium formate, pH 3:acetonitrile: 2-propanol (Fisher Scientific) and transferred to a 384-well microplate (Eppendorf) for analysis using LCMS.

For each batch, samples were randomized and matrix-matched reference quality controls and batch-specific pooled quality controls were included.

### Untargeted Analysis of Primary Metabolites and Biogenic Amines

Untargeted metabolomics analysis was conducted on Waters Acquity™ UPLC system with 2D column regeneration configuration (I-class and H-class) coupled to a Xevo G2-XS quadrupole time-of-flight (qTOF) mass spectrometer. Chromatographic separation was performed using HILIC (Acquity™ UPLC BEH amide, 100 Å, 1.7 μm 2.1×100 mm, Waters Corporation, Milford, U.S.A) and C18 (Acquity™ UPLC HSS T3, 100 Å, 1.8 μm, 2.1×100 mm, Water Corporation, Milford, U.S.A) columns at 45° C.

Quaternary solvent system mobile phases were (A) 0.1% formic acid in water, (B) 0.1% formic acid in acetonitrile and (D) 100 mM ammonium formate, pH 3. Samples were separated using the following gradient profile: for the HILIC separation a starting gradient of 95% B and 5% D was increase linearly to 70% A, 25% B and 5% D over a 5-min period at 0.4 mL/min flow rate, followed by 1 min isocratic gradient at 100% A at 0.4 mL/min flow rate. For C18 separation, a chromatography gradient was as follows: starting conditions, 100% A, with a linear increase to final conditions of 5% A, 95% B, followed by isocratic gradient at 95% B, 5% D for 1 min.

A binary pump was used for column regeneration and equilibration. The solvent system mobile phases were (A1) 100 mM ammonium formate, pH 3, (A2) 0.1% formic in 2-propanol and (B1) 0.1% formic acid in acetonitrile. The HILIC column was stripped using 90% A2 for 5 min followed by 2 min equilibration using 100% B1 at 0.3 mL/min flowrate. Reverse phase C18 column regeneration was performed using 95% A1, 5% B1 for 2 min followed by column equilibration using 5% A1, 95% B1 for 5 min.

### Untargeted Analysis of Complex Lipids

For the lipidomic assay, untargeted metabolomics analysis was conducted on a Waters Acquity™ UPLC system with 2D column regeneration configuration (I-class and H-class) coupled to a Xevo G2-XS quadrupole time-of-flight (qTOF) mass spectrometer. Chromatographic separation was performed using a C18 (Acquity™ UPLC HSS T3, 100 Å, 1.8 μm, 2.1×100 mm, Water Corporation, Milford, U.S.A) column at 55° C. The mobile phases were (A) water, (B) Acetonitrile, (C) 2-propanol and (D) 500 mM ammonium formate, pH 3. A starting elution gradient of 20% A, 30% B, 49% C, and 1% D was increased linearly to 10% B, 89% C and 1% D for 5.5 min, followed by isocratic elution at 10% B, 89% C and 1% D for 1.5 min and column equilibration with initial conditions for 1 min.

### Mass Spectrometry Data Acquisition

Mass spectrometry data was acquired in sensitivity, positive and negative electrospray ionization mode within 50-1200 Da range for primary metabolites and 100-2000 Da for complex lipids. For the electrospray acquisition, the capillary voltage was set at 1.5 kV (positive), 3.0 kV (negative), sample cone voltage of 30 V, source temperature of 120° C., cone gas flow of 50 L/h, and desolvation gas flow rate of 800 L/h with scan time of 0.5 sec in continuum mode. Leucine Enkephalin; 556.2771 Da (positive) and 554.2615 Da (negative) for lockspray correction and scans were performed at 0.5 min. The injection volume for each sample was 3 μL, unless otherwise specified. The acquisition was carried out with instrument auto gain control to optimize instrument sensitivity over the sample acquisition time.

Pooled quality control samples were analyzed after a defined number of samples to assess replicate precision and allow LOESS correction by injection order. Additional data was captured using the MSe function for pooled quality control samples.

### Data Processing

Peak picking and retention time alignment of LC-MS and MSe data were performed using Progenesis QI (Nonlinear, Waters). Data processing and peak annotations were performed using an in-house automated pipeline. Annotations were determined by matching accurate mass and retention times using customized libraries created from authentic standards and/or by matching experimental tandem mass spectrometry data against the NIST MSMS, LipidBlast or HMDB v3 theoretical fragmentations. To correct for injection order drift, each feature was normalized using data from repeat injections of quality control samples collected every 10 injections throughout the run sequence. Measurement data were smoothed by Locally Weighted Scatterplot Smoothing (LOESS) signal correction (QC-RLSC) as previously described (1). Only detected features exhibiting a relative standard deviation (RSD) less than 30 in quality control samples were considered for further statistical analysis. To reduce data matrix complexity, annotated features with multiple adducts or acquisition mode repeats were collapsed to one representative unique feature. Features were selected based on replicate precision (RSD<30), intensity and best isotope similarity matching to theoretical isotope distributions. Values are reported as ratios relative to the median of historical quality control reference samples ran with every analytical batch for the given analyte.

### Enzyme-Linked Immunosorbent Assay

Plasma protein concentrations for CA19-9, LRG1, and TIMP1 were determined as previously described (Capello et al., 2017). For all ELISA experiments, each sample was assayed in duplicate and the absorbance or chemiluminescence measured with a SpectraMax M5 microplate reader (Molecular Devices, Sunnyvale, CA). An internal control sample was run in every plate and each value of the samples was divided by the mean value of the internal control in the same plate to correct for interpolate variability.

### Gene Expression Data and Networks

Gene expression for the Badea dataset was downloaded from oncomine database. Networks were visualized using cytoscape.

### Statistical Analyses

Receiver operating characteristic (ROC) curve analysis was performed to assess the performance of biomarkers in distinguishing PDAC cases from healthy controls and subjects diagnosed with benign pancreatic disease (chronic pancreatitis or pancreatic cysts).

The AUC that corresponds to the individual performance of all biomarkers is estimated using the area under the empirical estimator of the receiver operating characteristic curve (ROC). The standard error (S.E.) and the corresponding 95% confidence intervals presented for the individual performance of each biomarker were based on the bootstrap procedure in which re-sampling was performed with replacement separately for the controls and the diseased 1000 bootstrap samples. It was noted that for markers LPC (18:0), LPC (20:3), and indole-3-lactate, the inverse directionality was taken into account, since these markers tend to exhibit higher measurements for the controls compared to the ones that correspond to the cancer related samples. The model building was based on a logistic regression model using the logit link function. The estimated AUC of the proposed metabolite panel (0.9034) was derived by using the empirical estimator of the linear combination that corresponds to the model. The 95% confidence interval reported for the metabolite panel based AUC (0.8180-0.9889) takes into account the fact that the coefficients of the underlying logistic regression model were estimated, and hence exhibit variability, by using the bootstrap with 1000 iterations, for which in every bootstrap iteration the coefficients of the model are re-estimated in order to provide proper inference. The hyper-panel, i.e. the panel that refers to the combination of the two underlying panels-one for the proteins and one for the metabolites—has been developed using those two panels as two composite markers, considering their respective coefficients fixed (one composite marker for the proteins and one for the metabolites). The hyper-panel was developed by combining those two underlying composite markers using a logistic regression model in which we considered the logit link function.

### Results

### Identification of Pancreatic Cancer Metabolite Biomarkers

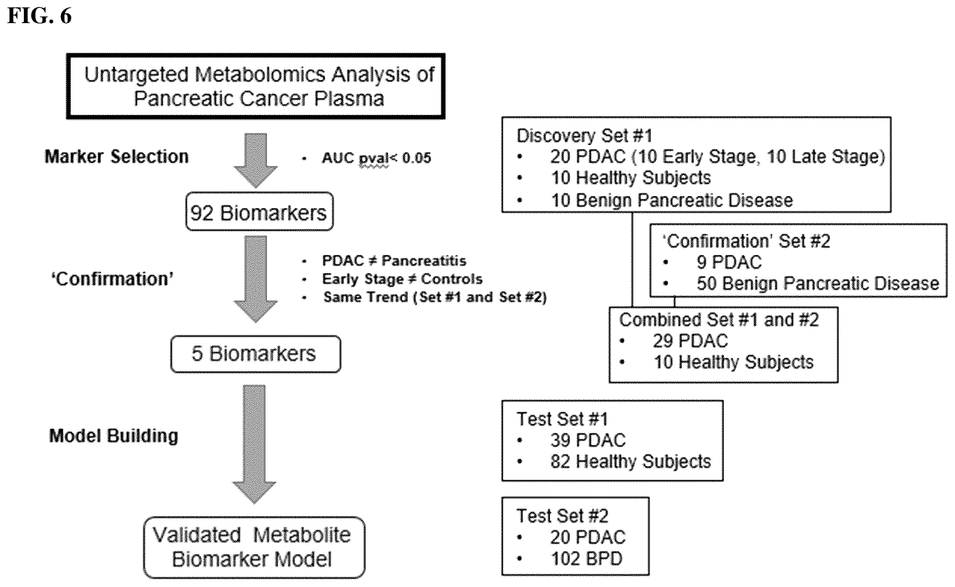

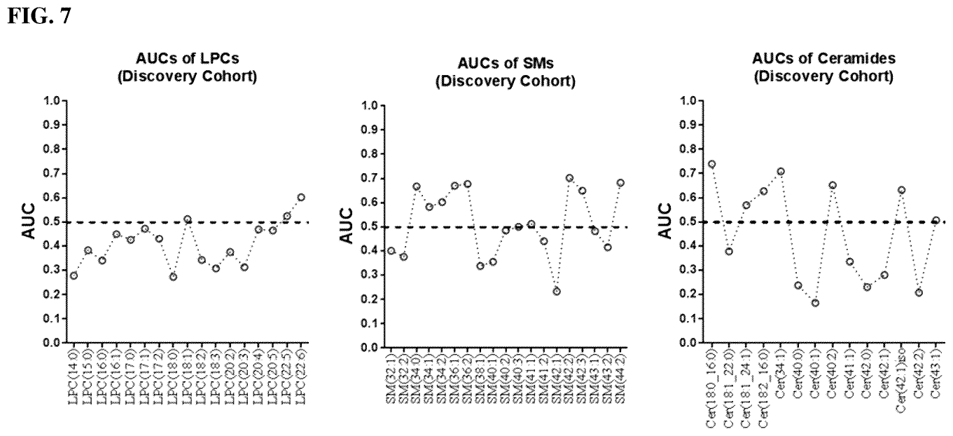

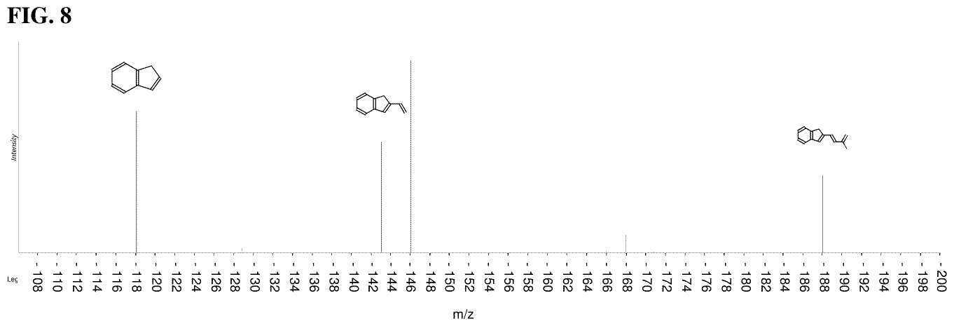

Untargeted metabolomics analysis was conducted on a discovery cohort (Set #1) consisting of 20 PDAC cases (10 early and 10 late stage) and 20 matched controls (10 healthy subjects and 10 subjects with chronic pancreatitis (CP) (FIG. 6). Candidate biomarkers were initially selected based on significant ROC AUCs (two-tailed Wilcox rank-sum Test<0.05) resulting in 91 metabolites (Table 15). To further narrow the candidate list, metabolomic analyses were conducted on an independent ‘confirmatory’ cohort (Set #2) consisting of 9 PDAC (5 early and 4 late stage) and 50 subjects with benign pancreatic disease (BPD) (benign pancreatic cysts). Of the 91 original features, 16 retained significant AUCs and maintained the same relative direction of change (increase/decrease) as observed in Set #1 (Table 16). Candidate metabolites were additionally refined to exclude (1) metabolites that exhibited similar levels between early-stage PDAC and subjects with CP (one-tailed Mann-Whitney U test p<0.1) and (2) metabolites that differed between CP and healthy controls (one-tailed Mann-Whitney U test p<0.1) (Table 16). In the case of individual lipid species, to mitigate non-specificity due to external factors such as dietary patterns, emphasis was given to those lipids that showed uniformity in the performance characteristics amongst the entire lipid class (i.e. >80% of the detected individual lipids in a given lipid class exhibited concordant increases/decreases in cases relative to controls (FIG. 7). A total of 5 metabolites were selected that met the aforementioned criteria. These five metabolites were (N1/N8)-acetylspermidine (AcSperm), diacetylspermine (DAS), lysophosphatidylcholine (LPC) (18:0). LPC (20:3), and an indole-derivative (FIG. 8 and Table 17).

TABLE 15
Ninety-two selected candidate metabolites based on discovery cohort.

| | | | | | | | Healthy′ | Chronic |
|---|---|---|---|---|---|---|---|---|
| | | | | | | Retention | Subjects | Pancreatitis |
| | | | Ionization | | | time | (Mean +/− | (Mean +/− |
| Index | CmpID | Adduct(s) | Mode | Assay | m/z | (min) | StDev) | StDev) |
| 5 | 1.2-DIDECANOYL-SN- | [M − | ESIpos | HA | 548.3685 | 0.9182 | 3.28 +/− 0.89 | 3.43 +/− 0.57 |
| | GLYCERO-3-PHOSPHOCHOLINE | H2O + H]+ | | | | | | |
| 23 | 3-(4-Hydroxyphenyl)propionic acid | [M − | ESIpos | HA | 149.0608 | 3.1457 | 0.54 +/− 0.05 | 0.58 +/− 0.04 |
| | | H2O + H]+ | | | | | | |
| 25 | 3-cis-Hydroxy-b,e-Caroten-3′-one | [M + NH4]+ | ESIpos | HA | 568.4561 | 0.7124 | 0.71 +/− 0.16 | 0.54 +/− 0.05 |
| 32 | 4-AMINOBENZOATE | [2M − H]− | ESIneg | CA | 273.0843 | 3.2229 | 0.98 +/− 0.17 | 0.99 +/− 0.28 |
| 47 | 7-oxo-cholesterol | [M + FA − | ESIneg | HA | 445.3313 | 0.678 | 1.11 +/− 0.7 | 0.51 +/− 0.2 |
| | | H]− | | | | | | |
| 48 | Acetylcarnitine | [M + H]+ | ESIpos | HA | 204.1242 | 3.1886 | 0.13 +/− 0.16 | 0.15 +/− 0.1 |
| 50 | Acylcarnitine(C14:0) | [M + H]+ | ESIpos | Lipids | 372.3114 | 0.7895 | 0.83 +/− 0.55 | 0.78 +/− 0.29 |
| 56 | ADENOSINE 5′- | [M + H]+ | ESIpos | CA | 348.0701 | 0.9267 | 0.14 +/− 0.16 | 0.08 +/− 0.02 |
| | MONOPHOSPHATE; AMP | | | | | | | |
| 57 | ADIPIC ACID | [M + H]+ | ESIpos | HA | 147.0653 | 0.7467 | 4.16 +/− 0.43 | 4.01 +/− 0.34 |
| 61 | ALPHA-D-GLUCOSE | [M + Na] | ESIpos | HA | 203.0538 | 3.4849 | 1 +/− 0.35 | 1.63 +/− 1.26 |
| 62 | Alpha-N-Phenylacetyl-L-glutamine | [M + H]+ | ESIpos | HA | 265.1193 | 2.8151 | 0.45 +/− 0.36 | 0.93 +/− 0.87 |
| 70 | BILIVERDIN | [M + H]+ | ESIpos | CA | 583.2509 | 4.3042 | 0.77 +/− 0.17 | 0.66 +/− 0.19 |
| 79 | Cer(18:0_16:0) | [M + Na]+ | ESIpos | Lipids | 562.517 | 3.987 | 0.59 +/− 0.12 | 0.81 +/− 0.24 |
| 83 | Cer(34:1) | [M − | ESIpos | Lipids | 520.5052 | 3.845 | 0.85 +/− 0.23 | 1.04 +/− 0.25 |
| | | H2O + H]+ | | | | | | |
| 84 | Cer(40:0) | [M + Na] | ESIpos | HA | 646.6123 | 0.618 | 2.21 +/− 0.33 | 1.64 +/− 0.41 |
| 85 | Cer(40:1) | IM − | ESIpos | HA | 604.5978 | 0.618 | 2.48 +/− 0.89 | 1.99 +/− 0.83 |
| | | H2O + H]+ | | | | | | |
| 88 | Cer(42:0) | [M + Na]+ | ESIpos | Lipids | 674.6387 | 5.377 | 0.79 +/− 0.29 | 0.78 +/− 0.23 |
| 89 | Cer(42:1) | [M − | ESIpos | Lipids | 632.6314 | 5.2913 | 0.79 +/− 0.32 | 0.9 +/− 0.36 |
| | | H2O + H]+ | | | | | | |
| 91 | Cer(42:2) | IM − | ESIpos | HA | 630.6156 | 0.618 | 2.77 +/− 0.89 | 2.02 +/− 0.5 |
| | | H2O + H]+ | | | | | | |
| 93 | Chavicol O-beta-glucopyranoside | [M − | ESIpos | HA | 279.1192 | 0.6781 | 1.41 +/− 0.21 | 1.24 +/− 0.11 |
| | | H2O + H]+ | | | | | | |
| 114 | D-(+)-GALACTOSAMINE; D-(+)- | [M + H]+ | ESIpos | CA | 162.0747 | 0.5104 | 1.28 +/− 0.22 | 1.45 +/− 0.77 |
| | GLUCOSAMINE; D- | | | | | | | |
| | MANNOSAMINE; N-METHYL-L- | | | | | | | |
| | GLUTAMATE | | | | | | | |
| 118 | DEOXYCORTICOSTERONE | [M + H]+ | ESIpos | HA | 373.2351 | 0.7381 | 6.06 +/− 0.85 | 6.51 +/− 0.49 |
| | ACETATE | | | | | | | |
| 119 | D-FRUCTOSE 6-PHOSPHATE | [M − H]− | ESIneg | CB | 259.0212 | 0.4418 | 1 +/− 0.11 | 1.07 +/− 0.1 |
| 121 | DG(34:0) | [M + Na]+ | ESIpos | Lipids | 619.5229 | 4.9092 | 0.69 +/− 0.09 | 0.76 +/− 0.18 |
| 124 | DG(35:1) | [M + NH4]+ | ESIpos | Lipids | 626.5788 | 4.4891 | 0.91 +/− 0.17 | 0.9 +/− 0.22 |
| 131 | DIACETYLSPERMINE | [M + H]+ | ESIpos | HA | 287.244 | 3.7164 | 0.85 +/− 0.13 | 0.93 +/− 0.15 |
| 138 | FA (20:4) (arachidonic acid) | [M − H]− | ESIneg | Lipids | 303.2323 | 1.4974 | 0.67 +/− 0.21 | 0.69 +/− 0.34 |
| 143 | GALACTITOL | [M + Na] | ESIpos | HA | 205.0684 | 3.4372 | 0.9 +/− 0.1 | 1.03 +/− 0.12 |
| 154 | GLUTATHIONE | [M + H]+ | ESIpos | CA | 308.0898 | 0.9696 | 6.8 +/− 2.67 | 6.94 +/− 2.98 |
| 155 | GLYCERALDEHYDE | [2M − H]− | ESIneg | CA | 179.0558 | 0.5323 | 0.71 +/− 0.17 | 1.02 +/− 0.61 |
| 156 | GLYCEROL 2-PHOSPHATE | [M − | ESIpos | CA | 155.0121 | 0.6266 | 1.11 +/− 0.14 | 1.12 +/− 0.15 |
| | | H2O + H]+ | | | | | | |
| 161 | GUANOSINE 3′.5′-CYCLIC | [M + H]+ | ESIpos | HA | 346.0572 | 3.7936 | 0.84 +/− 0.05 | 0.87 +/− 0.06 |
| | MONOPHOSPHATE | | | | | | | |
| 164 | Hexanoylcarnitine | [M + H]+ | ESIpos | HA | 260.1856 | 2.4978 | 0.72 +/− 0.22 | 0.9 +/− 0.43 |
| 168 | HOMOCYSTINE | [M − | ESIpos | HA | 251.0539 | 4.2614 | 1.7 +/− 0.67 | 1.25 +/− 0.14 |
| | | H2O + H]+ | | | | | | |
| 170 | Hydroxybutyrylcarnitine | [M + H]+ | ESIpos | HA | 248.1494 | 3.3086 | 1.21 +/− 0.97 | 0.84 +/− 0.29 |
| 172 | HYPOTAURINE | [2M − H]− | ESIneg | CA | 217.0298 | 0.5237 | 0.6 +/− 0.13 | 0.78 +/− 0.51 |
| 174 | INDOLE | [M + H]+ | ESIpos | HA | 118.0661 | 0.6781 | 2.09 +/− 0.53 | 1.55 +/− 0.18 |
| 177 | INDOLE-3-ETHANOL | [M − | ESIpos | HA | 144.0812 | 0.6781 | 2.14 +/− 0.61 | 1.6 +/− 0.22 |
| | | H2O + H]+ | | | | | | |
| 178 | Indole-derivative | [M − | ESIpos | HA | 188.0715 | 2.7208 | 0.65 +/− 0.9 | 0.78 +/− 1.18 |
| | | H2O + H]+ | | | | | | |
| 182 | INOSINE 5′-DIPHOSPHATE | [M − H]− | ESIneg | CB | 427.0055 | 0.4504 | 1.01 +/− 0.17 | 0.79 +/− 0.12 |
| 183 | INOSINE 5′-MONOPHOSPHATE | [M − | ESIneg | CA | 329.0323 | 0.781 | 0.77 +/− 0.13 | 0.74 +/− 0.09 |
| | | H2O − H]− | | | | | | |
| 184 | LacCer(30:1); PC(38:6) | [M + H]+ | ESIpos | Lipids | 806.5696 | 3.1714 | 0.99 +/− 0.4 | 0.82 +/− 0.31 |
| 192 | L-ARGININE | [M + H]+ | ESIpos | HA | 175.1199 | 4.1328 | 0.84 +/− 0.3 | 0.89 +/− 0.3 |
| 198 | L-CYSTINE | [M + H]+ | ESIpos | HA | 241.0311 | 4.39 | 1.69 +/− 0.5 | 1.93 +/− 0.49 |
| 201 | L-GLUTAMIC ACID | [M − H]− | ESIneg | CA | 146.0462 | 0.5104 | 0.27 +/− 0.13 | 0.34 +/− 0.23 |
| 209 | L-KYNURENINE | [M + | ESIneg | CB | 253.0814 | 1.4974 | 0.85 +/− 0.15 | 1.05 +/− 0.13 |
| | | FA − H]− | | | | | | |
| 215 | LPC(14:0) | [M + H]+ | ESIpos | HA | 468.3106 | 2.6779 | 0.28 +/− 0.11 | 0.45 +/− 0.22 |
| 223 | LPC(18:0) | [M + H]+ | ESIpos | Lipids | 524.3702 | 1.3088 | 0.38 +/− 0.17 | 0.44 +/− 0.23 |
| 226 | LPC(18:3) | [M + H]+ | ESIpos | Lipids | 518.3221 | 0.8324 | 0.41 +/− 0.21 | 0.44 +/− 0.25 |
| 227 | LPC(20:0) | [M + H]+ | ESIpos | HA | 552.4014 | 2.6007 | 0.36 +/− 0.11 | 0.59 +/− 0.41 |
| 230 | LPC(20:3) | [M + H]+ | ESIpos | CA | 546.355 | 5.2741 | 0.46 +/− 0.29 | 0.54 +/− 0.33 |
| 235 | LPC(26:0) | [M + H]+ | ESIpos | HA | 636.4901 | 2.5493 | 0.79 +/− 0.16 | 0.77 +/− 0.21 |
| 237 | LPC(P-16:0) | [M + H]+ | ESIpos | Lipids | 480.3433 | 1.1287 | 0.37 +/− 0.13 | 0.36 +/− 0.14 |
| 246 | LPE(22:6) | [M − H]− | ESIneg | CA | 524.2764 | 5.0598 | 0.69 +/− 0.44 | 0.45 +/− 0.26 |
| 250 | L-PHENYLALANINE | [M + H]+ | ESIpos | CA | 166.0865 | 1.8966 | 0.44 +/− 0.15 | 0.56 +/− 0.17 |
| 256 | MALTOSE; MELIBIOSE; SUCROSE | [M + Na] | ESIpos | HA | 365.1056 | 3.7678 | 0.48 +/− 0.09 | 0.73 +/− 0.23 |
| 261 | MELIBIOSE | [M + K]+ | ESIpos | HA | 381.0796 | 3.9136 | 0.73 +/− 0.2 | 1.16 +/− 0.22 |
| 267 | N8-ACETYLSPERMIDINE | [M + H]+ | ESIpos | HA | 188.1761 | 3.8793 | 0.98 +/− 0.28 | 0.93 +/− 0.34 |
| 272 | N-ACETYL-D-TRYPTOPHAN | [2M − H]− | ESIneg | HB | 491.1899 | 0.6952 | 0.84 +/− 0.22 | 0.92 +/− 0.08 |
| 278 | N-acetyllactosamine | [M + Na] | ESIpos | HA | 406.1332 | 3.7335 | 0.6 +/− 0.14 | 0.73 +/− 0.14 |
| 283 | NeuAc?2-3Gal?1-4Glc?- | [M + H]+ | ESIpos | Lipids | 1153.719 | 2.4502 | 0.83 +/− 0.28 | 0.86 +/− 0.18 |
| | Cer(d18:1/16:0) | | | | | | | |
| 289 | NG, NG-dimethyl-L-arginine | [M + H]+ | ESIpos | HA | 203.1507 | 3.905 | 0.74 +/− 0.12 | 0.81 +/− 0.13 |
| 294 | NICOTINAMIDE | [M + H]+ | ESIpos | CA | 123.0552 | 1.0601 | 0.75 +/− 0.5 | 0.47 +/− 0.1 |
| 300 | NICOTINAMIDE | [M − | ESIpos | CA | 317.0567 | 0.5495 | 1.43 +/− 0.1 | 1.35 +/− 0.31 |
| | MONONUCLEOTIDE | H2O + H]+ | | | | | | |
| 305 | N-METHYL-L-GLUTAMATE | [M − | ESIpos | CA | 144.0658 | 0.5104 | 1.23 +/− 0.21 | 1.35 +/− 0.67 |
| | | H2O + H]+ | | | | | | |
| 311 | Octanoylcarnitine | [M + H]+ | ESIpos | HA | 288.2182 | 2.3216 | 0.62 +/− 0.33 | 0.79 +/− 0.46 |
| 316 | PC(32:0) | [M + H]+ | ESIpos | Lipids | 734.5666 | 3.6221 | 0.78 +/− 0.22 | 0.96 +/− 0.29 |
| 322 | PC(33:5) | [M + H]+ | ESIpos | Lipids | 738.4968 | 3.4935 | 0.65 +/− 0.14 | 0.92 +/− 0.23 |
| 327 | PC(36:3) | [M + K]+ | ESIpos | Lipids | 822.5412 | 3.4849 | 0.95 +/− 0.11 | 1.02 +/− 0.1 |
| 333 | PC(40:5) | [M + Na]+ | ESIpos | Lipids | 858.5907 | 3.8965 | 1.08 +/− 0.36 | 1.14 +/− 0.3 |
| 338 | PC(o-42:5) or PC(p-42:4) | [M − | ESIpos | Lipids | 832.6594 | 4.5491 | 0.81 +/− 0.22 | 0.72 +/− 0.29 |
| | | H2O + H]+ | | | | | | |
| 344 | PE(37:4) | [M + H]+ | ESIpos | HA | 754.5378 | 2.03 | 0.53 +/− 0.14 | 0.53 +/− 0.27 |
| 349 | PE(41:3) | [M + | ESIneg | Lipids | 856.6023 | 3.9307 | 0.57 +/− 0.32 | 0.51 +/− 0.29 |
| | | FA − H]− | | | | | | |
| 355 | PE(o-36:5) or PE(p-36:4) | [M + H]+ | ESIpos | Lipids | 724.5217 | 3.6564 | 0.73 +/− 0.29 | 0.67 +/− 0.49 |
| 360 | PE(o-38:5) or PE(p-38:4) | [M + H]+ | ESIpos | Lipids | 752.5541 | 4.0727 | 0.9 +/− 0.35 | 0.66 +/− 0.36 |
| 366 | PHOSPHOCREATINE | [M + H]+ | ESIpos | CA | 212.041 | 0.5237 | 0.64 +/− 0.1 | 0.53 +/− 0.19 |
| 371 | PI(38:4) | [M + K]+ | ESIpos | HA | 925.5194 | 2.7293 | 1.37 +/− 0.5 | 1.65 +/− 0.44 |
| 377 | PS(o-18:0_22:6) | [M + Cl]− | ESIneg | Lipids | 856.5214 | 3.9307 | 0.58 +/− 0.32 | 0.49 +/− 0.27 |
| 382 | PYRIDOXINE | [M − | ESIneg | HA | 150.054 | 1.1887 | 0.72 +/− 0.27 | 0.99 +/− 1.26 |
| | | H2O − H]− | | | | | | |
| 388 | SM(42:1) | [M + H]+ | ESIpos | Lipids | 815.6967 | 4.8321 | 0.81 +/− 0.24 | 0.73 +/− 0.27 |
| 393 | SM(42:2) | [M + H]+ | ESIpos | Lipids | 813.6804 | 4.4414 | 0.84 +/− 0.28 | 0.74 +/− 0.16 |
| 399 | SM(44:2) | [M + H]+ | ESIpos | Lipids | 841.7163 | 4.8235 | 0.95 +/− 0.19 | 0.87 +/− 0.21 |
| 404 | TG(46:0) | [M + NH4]+ | ESIpos | Lipids | 796.7389 | 6.2526 | 0.5 +/− 0.24 | 0.76 +/− 0.46 |
| 410 | TG(47:0) | [M + NH4]+ | ESIpos | Lipids | 810.7538 | 6.3212 | 0.88 +/− 0.07 | 1.06 +/− 0.43 |
| 415 | TG(48:2) | [M + Na]+ | ESIpos | Lipids | 825.6948 | 6.0811 | 0.52 +/− 0.26 | 0.96 +/− 0.33 |
| 421 | TG(58:9) | [M + K]+ | ESIpos | Lipids | 967.7151 | 6.0297 | 1.02 +/− 0.34 | 0.76 +/− 0.22 |
| 426 | THEOBROMINE | [M + H]+ | ESIpos | CA | 181.0723 | 2.0129 | 0.45 +/− 0.49 | 0.61 +/− 0.49 |
| 432 | THYROXINE | [M + Na] | ESIpos | HA | 799.6671 | 2.4759 | 1.99 +/− 0.32 | 1.31 +/− 0.39 |
| 437 | toluene | [M + H]+ | ESIpos | HA | 93.0699 | 3.1543 | 0.26 +/− 0.09 | 0.32 +/− 0.07 |
| 443 | TRIGONELLINE | [M + H]+ | ESIpos | CA | 138.0554 | 0.5666 | 0.15 +/− 0.09 | 0.85 +/− 1.13 |
| 448 | TYRAMINE | [M − | ESIpos | CA | 120.0814 | 1.8966 | 0.47 +/− 0.14 | 0.57 +/− 0.17 |
| | | H2O + H]+ | | | | | | |

TABLE 15
Ninety-two selected candidate metabolites based on discovery cohort (cont.)

| | | Early | Late | | | | | | |
|---|---|---|---|---|---|---|---|---|---|
| | | Stage | Stage | Fold | | FDR- | | | |
| | | PDAC | PDAC | Change | Mann- | adjusted | | | |
| | | (Mean +/− | (Mean +/− | (PDAC/ | Whitney | p- | | Wilcox | AUC |
| Index | CmpID | Stdev) | Stdev) | Controls) | U test** | value*** | AUC** | T** | (Positive) |
| 5 | 1.2-DIDECANOYL-SN- | 3.68 +/− | 3.92 +/− | 1.14 | 0.0491 | 0.2444 | 0.6825 | 0.0491 | 0.6825 |
| | GLYCERO-3-PHOSPHOCHOLINE | 1.11 | 0.53 | | | | | | |
| 23 | 3-(4-Hydroxyphenyl)propionic acid | 0.63 +/− | 0.59 +/− | 1.08 | 0.0167 | 0.1389 | 0.7200 | 0.0167 | 0.7200 |
| | | 0.06 | 0.05 | | | | | | |
| 25 | 3-cis-Hydroxy-b,e-Caroten-3′-one | 0.52 +/− | 0.53 +/− | 0.84 | 0.0132 | 0.1178 | 0.2725 | 0.0132 | 0.7275 |
| | | 0.08 | 0.06 | | | | | | |
| 32 | 4-AMINOBENZOATE | 38.85 +/− | 1.78 +/− | 20.00 | 0.0167 | 0.1389 | 0.7200 | 0.0167 | 0.7200 |
| | | 89.38 | 1.7 | | | | | | |
| 47 | 7-oxo-cholesterol | 0.32 +/− | 0.46 +/− | 0.48 | 0.0016 | 0.0377 | 0.2150 | 0.0016 | 0.7850 |
| | | 0.14 | 0.33 | | | | | | |
| 48 | Acetylcarnitine | 0.3 +/− | 0.2 +/− | 1.79 | 0.0211 | 0.1558 | 0.7125 | 0.0211 | 0.7125 |
| | | 0.13 | 0.16 | | | | | | |
| 50 | Acylcarnitine(C14:0) | 1.21 +/− | 0.95 +/− | 1.35 | 0.0491 | 0.2444 | 0.6825 | 0.0491 | 0.6825 |
| | | 0.65 | 0.51 | | | | | | |
| 56 | ADENOSINE 5′- | 0.04 +/− | 0.07 +/− | 0.50 | 0.0095 | 0.1068 | 0.2625 | 0.0095 | 0.7375 |
| | MONOPHOSPHATE; AMP | 0.02 | 0.05 | | | | | | |
| 57 | ADIPIC ACID | 4.5 +/− | 4.35 +/− | 1.09 | 0.0350 | 0.2020 | 0.6950 | 0.0350 | 0.6950 |
| | | 0.61 | 0.39 | | | | | | |
| 61 | ALPHA-D-GLUCOSE | 1.59 +/− | 2.22 +/− | 1.45 | 0.0003 | 0.0156 | 0.8200 | 0.0003 | 0.8200 |
| | | 0.31 | 1.22 | | | | | | |
| 62 | Alpha-N-Phenylacetyl- | 1.12 +/− | 1.36 +/− | 1.82 | 0.0245 | 0.1653 | 0.7075 | 0.0245 | 0.7075 |
| | L-glutamine | 1.31 | 0.51 | | | | | | |
| 70 | BILIVERDIN | 1.85 +/− | 1.27 +/− | 2.17 | 0.0024 | 0.0515 | 0.7750 | 0.0024 | 0.7750 |
| | | 1.97 | 1.17 | | | | | | |
| 79 | Cer(18:0_16:0) | 2.04 +/− | 0.92 +/− | 2.13 | 0.0087 | 0.1068 | 0.7400 | 0.0087 | 0.7400 |
| | | 2.87 | 0.33 | | | | | | |
| 83 | Cer(34:1) | 2.41 +/− | 1.25 +/− | 1.92 | 0.0227 | 0.1653 | 0.7100 | 0.0227 | 0.7100 |
| | | 2.81 | 0.48 | | | | | | |
| 84 | Cer(40:0) | 2.38 +/− | 1.37 +/− | 0.97 | 0.0039 | 0.0613 | 0.2375 | 0.0039 | 0.7625 |
| | | 2.62 | 0.4 | | | | | | |
| 85 | Cer(40:1) | 2.68 +/− | 0.95 +/− | 0.81 | 0.0002 | 0.0092 | 0.1650 | 0.0002 | 0.8350 |
| | | 4.42 | 0.27 | | | | | | |
| 88 | Cer(42:0) | 0.66 +/− | 0.51 +/− | 0.74 | 0.0029 | 0.0584 | 0.2300 | 0.0029 | 0.7700 |
| | | 0.42 | 0.24 | | | | | | |
| 89 | Cer(42:1) | 0.66 +/− | 0.53 +/− | 0.70 | 0.0167 | 0.1389 | 0.2800 | 0.0167 | 0.7200 |
| | | 0.31 | 0.24 | | | | | | |
| 91 | Cer(42:2) | 3.71 +/− | 1.47 +/− | 1.08 | 0.0012 | 0.0323 | 0.2075 | 0.0012 | 0.7925 |
| | | 5.85 | 0.43 | | | | | | |
| 93 | Chavicol O-beta-glucopyranoside | 1.17 +/− | 1.08 +/− | 0.85 | 0.0001 | 0.0081 | 0.1550 | 0.0001 | 0.8450 |
| | | 0.11 | 0.1 | | | | | | |
| 114 | D-(+)-GALACTOSAMINE; D-(+)- | 1.58 +/− | 1.99 +/− | 1.32 | 0.0014 | 0.0357 | 0.7875 | 0.0014 | 0.7875 |
| | GLUCOSAMINE; D- | 0.3 | 0.85 | | | | | | |
| | MANNOSAMINE; N-METHYL-L- | | | | | | | | |
| | GLUTAMATE | | | | | | | | |
| 118 | DEOXYCORTICOSTERONE | 6.58 +/− | 7.19 +/− | 1.10 | 0.0103 | 0.1109 | 0.7350 | 0.0103 | 0.7350 |
| | ACETATE | 0.72 | 0.61 | | | | | | |
| 119 | D-FRUCTOSE 6-PHOSPHATE | 0.68 +/− | 0.75 +/− | 0.69 | 0.0014 | 0.0357 | 0.2125 | 0.0014 | 0.7875 |
| | | 0.41 | 0.37 | | | | | | |
| 121 | DG(34:0) | 0.62 +/− | 0.57 +/− | 0.82 | 0.0009 | 0.0294 | 0.2025 | 0.0009 | 0.7975 |
| | | 0.13 | 0.1 | | | | | | |
| 124 | DG(35:1) | 0.76 +/− | 0.77 +/− | 0.85 | 0.0103 | 0.1109 | 0.2650 | 0.0103 | 0.7350 |
| | | 0.16 | 0.11 | | | | | | |
| 131 | DIACETYLSPERMINE | 1.18 +/− | 1 +/− | 1.23 | 0.0112 | 0.1128 | 0.7325 | 0.0112 | 0.7325 |
| | | 0.43 | 0.24 | | | | | | |
| 138 | FA (20:4) (arachidonic acid) | 0.82 +/− | 0.98 +/− | 1.33 | 0.0375 | 0.2040 | 0.6925 | 0.0375 | 0.6925 |
| | | 0.38 | 0.3 | | | | | | |
| 143 | GALACTITOL | 1.01 +/− | 1.06 +/− | 1.08 | 0.0402 | 0.2159 | 0.6900 | 0.0402 | 0.6900 |
| | | 0.07 | 0.12 | | | | | | |
| 154 | GLUTATHIONE | 5.56 +/− | 4.09 +/− | 0.70 | 0.0073 | 0.0963 | 0.2550 | 0.0073 | 0.7450 |
| | | 3.2 | 2 | | | | | | |
| 155 | GLYCERALDEHYDE | 0.89 +/− | 1.18 +/− | 1.19 | 0.0073 | 0.0963 | 0.7450 | 0.0073 | 0.7450 |
| | | 0.23 | 0.36 | | | | | | |
| 156 | GLYCEROL 2-PHOSPHATE | 0.98 +/− | 1.02+/− | 0.90 | 0.0263 | 0.1706 | 0.2950 | 0.0263 | 0.7050 |
| | | 0.12 | 0.15 | | | | | | |
| 161 | GUANOSINE 3′.5′-CYCLIC | 0.53 +/− | 0.64 +/− | 0.68 | 0.0112 | 0.1128 | 0.2675 | 0.0112 | 0.7325 |
| | MONOPHOSPHATE | 0.37 | 0.38 | | | | | | |
| 164 | Hexanoylcarnitine | 1.52 +/− | 1.28 +/− | 1.72 | 0.0122 | 0.1174 | 0.7300 | 0.0122 | 0.7300 |
| | | 0.75 | 0.88 | | | | | | |
| 168 | HOMOCYSTINE | 1.17 +/− | 1.27 +/− | 0.83 | 0.0491 | 0.2444 | 0.3175 | 0.0491 | 0.6825 |
| | | 0.17 | 0.21 | | | | | | |
| 170 | Hydroxybutyrylcarnitine | 2.08 +/− | 2.38 +/− | 2.17 | 0.0132 | 0.1178 | 0.7275 | 0.0132 | 0.7275 |
| | | 1.95 | 2.39 | | | | | | |
| 172 | HYPOTAURINE | 0.77 +/− | 0.98 +/− | 1.27 | 0.0039 | 0.0613 | 0.7625 | 0.0039 | 0.7625 |
| | | 0.15 | 0.34 | | | | | | |
| 174 | INDOLE | 1.34 +/− | 1.21 +/− | 0.70 | 0.0000 | 0.0004 | 0.0925 | 0.0000 | 0.9075 |
| | | 0.15 | 0.21 | | | | | | |
| 177 | INDOLE-3-ETHANOL | 1.37 +/− | 1.23 +/− | 0.69 | 0.0000 | 0.0015 | 0.1175 | 0.0000 | 0.8825 |
| | | 0.18 | 0.2 | | | | | | |
| 178 | Indole-derivative | 0.21 +/− | 0.42 +/− | 0.44 | 0.0283 | 0.1762 | 0.2975 | 0.0283 | 0.7025 |
| | | 0.23 | 0.85 | | | | | | |
| 182 | INOSINE 5′-DIPHOSPHATE | 0.57 +/− | 0.66 +/− | 0.68 | 0.0181 | 0.1426 | 0.2825 | 0.0181 | 0.7175 |
| | | 0.35 | 0.37 | | | | | | |
| 183 | INOSINE 5′-MONOPHOSPHATE | 0.57 +/− | 0.55 +/− | 0.74 | 0.0211 | 0.1558 | 0.2875 | 0.0211 | 0.7125 |
| | | 0.35 | 0.27 | | | | | | |
| 184 | LacCer(30:1); PC(38:6) | 1.26 +/− | 1.11 +/− | 1.30 | 0.0245 | 0.1653 | 0.7075 | 0.0245 | 0.7075 |
| | | 0.65 | 0.31 | | | | | | |
| 192 | L-ARGININE | 1.16 +/− | 1.19 +/− | 1.35 | 0.0245 | 0.1653 | 0.7075 | 0.0245 | 0.7075 |
| | | 0.54 | 0.46 | | | | | | |
| 198 | L-CYSTINE | 2.4 +/− | 2.6 +/− | 1.39 | 0.0012 | 0.0323 | 0.7925 | 0.0012 | 0.7925 |
| | | 0.59 | 0.88 | | | | | | |
| 201 | L-GLUTAMIC ACID | 0.5 +/− | 0.45 +/− | 1.56 | 0.0304 | 0.1844 | 0.7000 | 0.0304 | 0.7000 |
| | | 0.41 | 0.23 | | | | | | |
| 209 | L-KYNURENINE | 1.1 +/− | 1.09 +/− | 1.15 | 0.0304 | 0.1844 | 0.7000 | 0.0304 | 0.7000 |
| | | 0.22 | 0.19 | | | | | | |
| 215 | LPC(14:0) | 0.23 +/− | 0.23 +/− | 0.63 | 0.0155 | 0.1356 | 0.2775 | 0.0155 | 0.7225 |
| | | 0.1 | 0.13 | | | | | | |
| 223 | LPC(18:0) | 0.24 +/− | 0.26 +/− | 0.62 | 0.0132 | 0.1178 | 0.2725 | 0.0132 | 0.7275 |
| | | 0.11 | 0.1 | | | | | | |
| 226 | LPC(18:3) | 0.27 +/− | 0.28 +/− | 0.65 | 0.0375 | 0.2040 | 0.3075 | 0.0375 | 0.6925 |
| | | 0.14 | 0.12 | | | | | | |
| 227 | LPC(20:0) | 0.35 +/− | 0.32 +/− | 0.70 | 0.0350 | 0.2020 | 0.3050 | 0.0350 | 0.6950 |
| | | 0.3 | 0.12 | | | | | | |
| 230 | LPC(20:3) | 0.32 +/− | 0.32 +/− | 0.64 | 0.0430 | 0.2259 | 0.3125 | 0.0430 | 0.6875 |
| | | 0.14 | 0.17 | | | | | | |
| 235 | LPC(26:0) | 0.59 +/− | 0.57 +/− | 0.75 | 0.0009 | 0.0294 | 0.2025 | 0.0009 | 0.7975 |
| | | 0.14 | 0.13 | | | | | | |
| 237 | LPC(P-16:0) | 0.24 +/− | 0.3 +/− | 0.74 | 0.0263 | 0.1706 | 0.2950 | 0.0263 | 0.7050 |
| | | 0.11 | 0.14 | | | | | | |
| 246 | LPE(22:6) | 1 +/− | 0.92 +/− | 1.67 | 0.0087 | 0.1068 | 0.7400 | 0.0087 | 0.7400 |
| | | 0.45 | 0.8 | | | | | | |
| 250 | L-PHENYLALANINE | 0.65 +/− | 0.62 +/− | 1.28 | 0.0181 | 0.1426 | 0.7175 | 0.0181 | 0.7175 |
| | | 0.25 | 0.13 | | | | | | |
| 256 | MALTOSE; MELIBIOSE; SUCROSE | 7.54 +/− | 0.85 +/− | 7.14 | 0.0283 | 0.1762 | 0.7025 | 0.0283 | 0.7025 |
| | | 21.42 | 0.51 | | | | | | |
| 261 | MELIBIOSE | 3.39 +/− | 2.52 +/− | 3.13 | 0.0001 | 0.0081 | 0.8450 | 0.0001 | 0.8450 |
| | | 4.54 | 3.11 | | | | | | |
| 267 | N8-ACETYLSPERMIDINE | 1.76 +/− | 1.59 +/− | 1.75 | 0.0043 | 0.0631 | 0.7600 | 0.0043 | 0.7600 |
| | | 1.04 | 0.7 | | | | | | |
| 272 | N-ACETYL-D-TRYPTOPHAN | 1.18 +/− | 1.11 +/− | 1.30 | 0.0263 | 0.1706 | 0.7050 | 0.0263 | 0.7050 |
| | | 0.35 | 0.43 | | | | | | |
| 278 | N-acetyllactosamine | 0.89 +/− | 0.74 +/− | 1.22 | 0.0375 | 0.2040 | 0.6925 | 0.0375 | 0.6925 |
| | | 0.26 | 0.16 | | | | | | |
| 283 | NeuAc?2-3Gal?1-4Glc?- | 1.39 +/− | 1.32 +/− | 1.61 | 0.0032 | 0.0584 | 0.7675 | 0.0032 | 0.7675 |
| | Cer(d18:1/16:0) | 0.8 | 0.59 | | | | | | |
| 289 | NG, NG-dimethyl-L-arginine | 1.09 +/− | 0.94 +/− | 1.32 | 0.0073 | 0.0963 | 0.7450 | 0.0073 | 0.7450 |
| | | 0.45 | 0.25 | | | | | | |
| 294 | NICOTINAMIDE | 0.37 +/− | 0.35 +/− | 0.60 | 0.0006 | 0.0217 | 0.1925 | 0.0006 | 0.8075 |
| | | 0.12 | 0.13 | | | | | | |
| 300 | NICOTINAMIDE | 0.84 +/− | 0.95 +/− | 0.65 | 0.0181 | 0.1426 | 0.2825 | 0.0181 | 0.7175 |
| | MONONUCLEOTIDE | 0.73 | 0.65 | | | | | | |
| 305 | N-METHYL-L-GLUTAMATE | 1.56 +/− | 1.89 +/− | 1.33 | 0.0001 | 0.0081 | 0.8400 | 0.0001 | 0.8400 |
| | | 0.28 | 0.78 | | | | | | |
| 311 | Octanoylcarnitine | 1.23 +/− | 1.52 +/− | 1.96 | 0.0350 | 0.2020 | 0.6950 | 0.0350 | 0.6950 |
| | | 0.76 | 1.72 | | | | | | |
| 316 | PC(32:0) | 2.03 +/− | 1.11 +/− | 1.82 | 0.0375 | 0.2040 | 0.6925 | 0.0375 | 0.6925 |
| | | 2.24 | 0.38 | | | | | | |
| 322 | PC(33:5) | 1.33 +/− | 1.13 +/− | 1.56 | 0.0043 | 0.0631 | 0.7600 | 0.0043 | 0.7600 |
| | | 0.84 | 0.37 | | | | | | |
| 327 | PC(36:3) | 0.9 +/− | 0.94 +/− | 0.93 | 0.0245 | 0.1653 | 0.2925 | 0.0245 | 0.7075 |
| | | 0.12 | 0.07 | | | | | | |
| 333 | PC(40:5) | 0.9 +/− | 0.76 +/− | 0.75 | 0.0035 | 0.0613 | 0.2350 | 0.0035 | 0.7650 |
| | | 0.28 | 0.25 | | | | | | |
| 338 | PC(o-42:5) or PC(p-42:4) | 1.36 +/− | 1.25 +/− | 1.69 | 0.0024 | 0.0515 | 0.7750 | 0.0024 | 0.7750 |
| | | 0.72 | 0.62 | | | | | | |
| 344 | PE(37:4) | 0.36 +/− | 0.38 +/− | 0.69 | 0.0132 | 0.1178 | 0.2725 | 0.0132 | 0.7275 |
| | | 0.15 | 0.19 | | | | | | |
| 349 | PE(41:3) | 0.72 +/− | 0.84 +/− | 1.45 | 0.0122 | 0.1174 | 0.7300 | 0.0122 | 0.7300 |
| | | 0.32 | 0.38 | | | | | | |
| 355 | PE(o-36:5) or PE(p-36:4) | 0.41 +/− | 0.48 +/− | 0.63 | 0.0095 | 0.1068 | 0.2625 | 0.0095 | 0.7375 |
| | | 0.3 | 0.19 | | | | | | |
| 360 | PE(o-38:5) or PE(p-38:4) | 0.43 +/− | 0.52 +/− | 0.61 | 0.0095 | 0.1068 | 0.2625 | 0.0095 | 0.7375 |
| | | 0.3 | 0.21 | | | | | | |
| 366 | PHOSPHOCREATINE | 0.39 +/− | 0.44 +/− | 0.70 | 0.0039 | 0.0613 | 0.2375 | 0.0039 | 0.7625 |
| | | 0.18 | 0.15 | | | | | | |
| 371 | PI(38:4) | 1.04 +/− | 0.87 +/− | 0.63 | 0.0005 | 0.0186 | 0.1875 | 0.0005 | 0.8125 |
| | | 0.4 | 0.2 | | | | | | |
| 377 | PS(o-18:0_22:6) | 0.69 +/− | 0.83 +/− | 1.43 | 0.0195 | 0.1515 | 0.7150 | 0.0195 | 0.7150 |
| | | 0.31 | 0.43 | | | | | | |
| 382 | PYRIDOXINE | 2.3 +/− | 4.85 +/− | 4.17 | 0.0067 | 0.0962 | 0.7475 | 0.0067 | 0.7475 |
| | | 3.89 | 5.78 | | | | | | |
| 388 | SM(42:1) | 0.54 +/− | 0.59 +/− | 0.72 | 0.0032 | 0.0584 | 0.2325 | 0.0032 | 0.7675 |
| | | 0.18 | 0.16 | | | | | | |
| 393 | SM(42:2) | 1.03 +/− | 1.12 +/− | 1.37 | 0.0283 | 0.1762 | 0.7025 | 0.0283 | 0.7025 |
| | | 0.47 | 0.47 | | | | | | |
| 399 | SM(44:2) | 1.04 +/− | 1.14 +/− | 1.19 | 0.0491 | 0.2444 | 0.6825 | 0.0491 | 0.6825 |
| | | 0.28 | 0.35 | | | | | | |
| 404 | TG(46:0) | 0.4 +/− | 0.45 +/− | 0.68 | 0.0375 | 0.2040 | 0.3075 | 0.0375 | 0.6925 |
| | | 0.08 | 0.22 | | | | | | |
| 410 | TG(47:0) | 0.83 +/− | 0.89 +/− | 0.88 | 0.0350 | 0.2020 | 0.3050 | 0.0350 | 0.6950 |
| | | 0.08 | 0.17 | | | | | | |
| 415 | TG(48:2) | 0.54 +/− | 0.38 +/− | 0.62 | 0.0245 | 0.1653 | 0.2925 | 0.0245 | 0.7075 |
| | | 0.26 | 0.14 | | | | | | |
| 421 | TG(58:9) | 1.26 +/− | 1.4 +/− | 1.49 | 0.0032 | 0.0584 | 0.7675 | 0.0032 | 0.7675 |
| | | 0.46 | 0.5 | | | | | | |
| 426 | THEOBROMINE | 0.12 +/− | 0.26 +/− | 0.36 | 0.0112 | 0.1128 | 0.2675 | 0.0112 | 0.7325 |
| | | 0.13 | 0.39 | | | | | | |
| 432 | THYROXINE | 1 +/− | 1.17 +/− | 0.66 | 0.0002 | 0.0120 | 0.1725 | 0.0002 | 0.8275 |
| | | 0.31 | 0.19 | | | | | | |
| 437 | toluene | 0.38 +/− | 0.34 +/− | 1.22 | 0.0460 | 0.2389 | 0.6850 | 0.0460 | 0.6850 |
| | | 0.11 | 0.09 | | | | | | |
| 443 | TRIGONELLINE | 0.71 +/− | 1.34 +/− | 2.04 | 0.0430 | 0.2259 | 0.6875 | 0.0430 | 0.6875 |
| | | 0.84 | 2.15 | | | | | | |
| 448 | TYRAMINE | 0.66 +/− | 0.64 +/− | 1.25 | 0.0211 | 0.1558 | 0.7125 | 0.0211 | 0.7125 |
| | | 0.25 | 0.13 | | | | | | |

TABLE 16
Sixteen candidate metabolites that indicated significant AUCs in both the discovery cohort and ‘confirmatory’ cohort.

| | | | | | | Set #1 | | Early |
|---|---|---|---|---|---|---|---|---|
| | | | | | AUC | Healthy′ | Chronic | Stage |
| | | | | | Direction | Subjects | Pancreatitis | PDAC |
| Set #1 | | Wilcox | Set #2 | Wilcox | (Set#1 vs | (Mean +/− | (Mean +/− | (Mean +/− |
| CmpID | AUC** | T** | AUC** | T** | Set#2) | StDev) | StDev) | Stdev) |
| ALPHA-D-GLUCOSE | 0.8200 | 0.0003 | 0.7178 | 0.0398 | same | 1 +/− 0.35 | 1.63 +/− 1.26 | 1.59 +/− 0.31 |
| Alpha-N-Phenylacetyl- | 0.7075 | 0.0245 | 0.7844 | 0.0072 | same | 0.45 +/− 0.36 | 0.93 +/− 0.87 | 1.12 +/− 1.31 |
| L-glutamine | | | | | | | | |
| Cer(40:1) | 0.1650 | 0.0002 | 0.2778 | 0.0359 | same | 2.48 +/− 0.89 | 1.99 +/− 0.83 | 2.68 +/− 4.42 |
| Cer(42:0) | 0.2300 | 0.0029 | 0.1867 | 0.0031 | same | 0.79 +/− 0.29 | 0.78 +/− 0.23 | 0.66 +/− 0.42 |
| Cer(42:1) | 0.2800 | 0.0167 | 0.2578 | 0.0222 | same | 0.79 +/− 0.32 | 0.9 +/− 0.36 | 0.66 +/− 0.31 |
| Cer(42:2) | 0.2075 | 0.0012 | 0.2600 | 0.0234 | same | 2.77 +/− 0.89 | 2.02 +/− 0.5 | 3.71 +/− 5.85 |
| DG(35:1) | 0.2650 | 0.0103 | 0.1422 | 0.0007 | same | 0.91 +/− 0.17 | 0.9 +/− 0.22 | 0.76 +/− 0.16 |
| INDOLE | 0.0925 | 0.0000 | 0.2800 | 0.0378 | same | 2.09 +/− 0.53 | 1.55 +/− 0.18 | 1.34 +/− 0.15 |
| Indole-derivative | 0.2975 | 0.0283 | 0.2622 | 0.0248 | same | 0.65 +/− 0.9 | 0.78 +/− 1.18 | 0.21 +/− 0.23 |
| LPC(18:0) | 0.2725 | 0.0132 | 0.2489 | 0.0177 | same | 0.38 +/− 0.17 | 0.44 +/− 0.23 | 0.24 +/− 0.11 |
| LPC(20:3) | 0.3125 | 0.0430 | 0.1756 | 0.0022 | same | 0.46 +/− 0.29 | 0.54 +/− 0.33 | 0.32 +/− 0.14 |
| N8- | 0.7600 | 0.0043 | 0.7133 | 0.0441 | same | 0.98 +/− 0.28 | 0.93 +/− 0.34 | 1.76 +/− 1.04 |
| ACETYLSPERMIDINE | | | | | | | | |
| N-acetyllactosamine | 0.6925 | 0.0375 | 0.7244 | 0.0341 | same | 0.6 +/− 0.14 | 0.73 +/− 0.14 | 0.89 +/− 0.26 |
| SM(42:1) | 0.2325 | 0.0032 | 0.2111 | 0.0063 | same | 0.81 +/− 0.24 | 0.73 +/− 0.27 | 0.54 +/− 0.18 |
| THYROXINE | 0.1725 | 0.0002 | 0.2867 | 0.0441 | same | 1.99 +/− 0.32 | 1.31 +/− 0.39 | 1 +/− 0.31 |
| DIACETYLSPERMINE | 0.7325 | 0.0112 | 0.7843 | 0.0069 | same | 0.85 +/− 0.13 | 0.93 +/− 0.15 | 1.18 +/− 0.43 |

| | Late | Fold | | | | | | |
|---|---|---|---|---|---|---|---|---|
| | Stage | Change | 2-sided | 1-sided | Fold | 2-sided | 1-sided | |
| | PDAC | (early | Mann- | Mann- | Change | Mann- | Mann- | |
| Set #1 | (Mean +/− | stage PDAC | Whitney | Whitney | (CP vs | Whitney | Whitney | |
| CmpID | Stdev) | vs CP) | U Test | U Test | Healthy) | U Test | U Test | Selected |
| ALPHA-D-GLUCOSE | 2.22 +/− 1.22 | 0.97 | 0.1431 | 0.0716 | 1.63 | 0.0887 | 0.0444 | Excluded |
| Alpha-N-Phenylacetyl- | 1.36 +/− 0.51 | 1.21 | 0.9118 | 0.4559 | 2.08 | 0.2150 | 0.1075 | Excluded |
| L-glutamine | | | | | | | | |
| Cer(40:1) | 0.95 +/− 0.27 | 1.35 | 0.1903 | 0.0952 | 0.80 | 0.3104 | 0.1552 | Excluded |
| Cer(42:0) | 0.51 +/− 0.24 | 0.84 | 0.1051 | 0.0526 | 0.99 | 0.7796 | 0.3898 | Excluded |
| Cer(42:1) | 0.53 +/− 0.24 | 0.74 | 0.1230 | 0.0615 | 1.14 | 0.4285 | 0.2143 | Excluded |
| Cer(42:2) | 1.47 +/− 0.43 | 1.83 | 0.1903 | 0.0952 | 0.73 | 0.0288 | 0.0144 | Excluded |
| DG(35:1) | 0.77 +/− 0.11 | 0.84 | 0.1431 | 0.0716 | 0.99 | 0.7796 | 0.3898 | Excluded |
| INDOLE | 1.21 +/− 0.21 | 0.87 | 0.0232 | 0.0116 | 0.74 | 0.0068 | 0.0034 | Excluded |
| Indole-derivative | 0.42 +/− 0.85 | 0.27 | 0.0753 | 0.0376 | 1.19 | 0.8928 | 0.4464 | Included |
| LPC(18:0) | 0.26 +/− 0.1 | 0.55 | 0.0355 | 0.0177 | 1.16 | 0.4727 | 0.2364 | Included |
| LPC(20:3) | 0.32 +/− 0.17 | 0.59 | 0.1230 | 0.0615 | 1.18 | 0.5678 | 0.2839 | Included |
| N8- | 1.59 +/− 0.7 | 1.89 | 0.0630 | 0.0315 | 0.95 | 0.5678 | 0.2839 | Included |
| ACETYLSPERMIDINE | | | | | | | | |
| N-acetyllactosamine | 0.74 +/− 0.16 | 1.22 | 0.1903 | 0.0952 | 1.22 | 0.0627 | 0.0314 | Excluded |
| SM(42:1) | 0.59 +/− 0.16 | 0.73 | 0.0630 | 0.0315 | 0.90 | 0.8928 | 0.4464 | Excluded |
| THYROXINE | 1.17 +/− 0.19 | 0.76 | 0.0892 | 0.0446 | 0.66 | 0.0007 | 0.0004 | Excluded |
| DIACETYLSPERMINE | 1 +/− 0.24 | 1.89 | 0.0627 | 0.0314 | 1.10 | 0.1220 | 0.0610 | Included |

TABLE 17
Selected metabolite marker performance in discovery and confirmatory cohorts.

| | Discovery Set | | | Confirmatory Set | | | Combined Set | | |
|---|---|---|---|---|---|---|---|---|---|
| Metabolite | FC¹ | AUC | p-value^{&} | FC² | AUC | p-value^{&} | FC³ | AUC | p-value^{&} |
| Indole-derivative^{#} | 0.44 | 0.7 | 0.0142 | 0.46 | 0.74 | 0.0124 | 0.54 | 0.61 | 0.1596 |
| LPC (18:0)^{#} | 0.62 | 0.73 | 0.0066 | 0.63 | 0.75 | 0.0089 | 0.59 | 0.76 | 0.0014 |
| LPC (20:3)^{#} | 0.64 | 0.69 | 0.0215 | 0.56 | 0.82 | 0.0011 | 0.65 | 0.68 | 0.0453 |
| ACETYLSPERMIDINE | 1.75 | 0.76 | 0.0022 | 1.38 | 0.71 | 0.0221 | 1.81 | 0.79 | <0.001 |
| DIACETYLSPERMINE | 1.23 | 0.73 | 0.0056 | 1.06 | 0.78 | 0.0035 | 1.25 | 0.8 | <0.001 |

¹fold change depicting PDAC (n = 20) relative to controls (10 healthy subjects; 10 subjects with chronic pancreatitis)
²fold change depicting PDAC (n = 9) relative to BPD (n = 50)
³fold change depicting PDAC (n = 29) relative to Healthy subjects (n = 10)
^{#}AUCs < 0.5 are flipped
^{&}one-tailed p-values, specify test
Abbrev. LPC: lysophosphatidylcholine

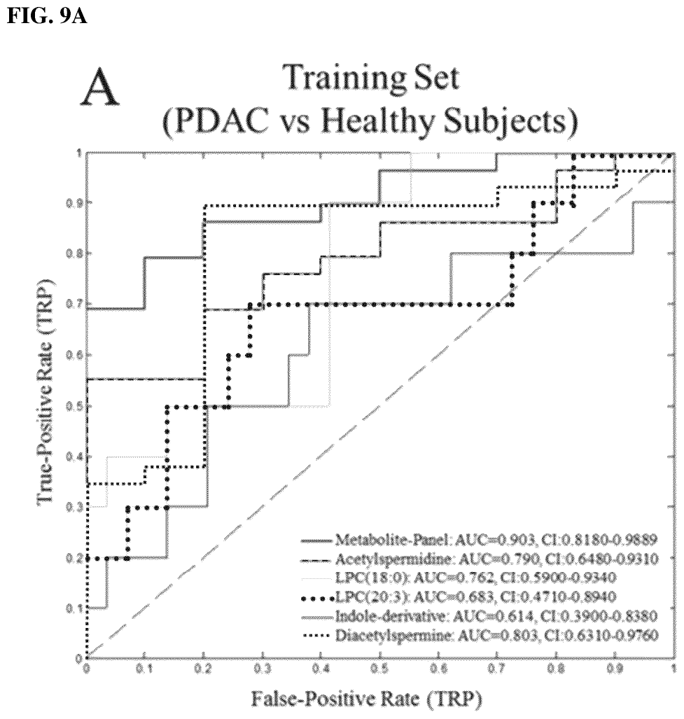

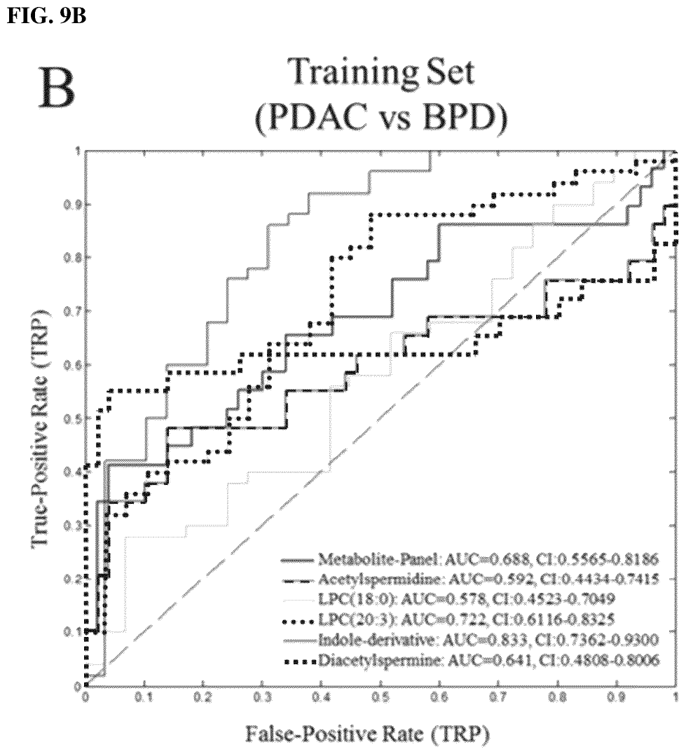

Next, a biomarker panel for PDAC was developed based on a logistic regression model. PDAC cases (n=29) from Set #1 and #2 were combined and evaluated against healthy subjects (n=10) from Set #1 (FIG. 7). Estimated coefficients as obtained by a logistic regression model that incorporates a logit link function are provided in Table 18. Individual performances of the 5 metabolite markers for the combined dataset are provided in Table 17. In comparison of PDAC with healthy subjects, the resulting panel of AcSperm+DAS+LPC (18:0)+LPC (20:3)+indole-derivative yielded an AUC of 0.90 (95% C.I.=0.818-0.989), exhibiting 69% sensitivity at 99% specificity (FIG. 9A). Performance of the metabolite panel for differentiating PDAC from BPD (chronic pancreatitis and low-grade cysts) yielded an AUC of 0.69 (95% C.I.=0.557-0.819) with 41% sensitivity at 95% specificity; however, the greatest achieved AUC was obtained with the indole-derivative alone (AUC-0.833) (FIG. 9B).

### Testing of Metabolite Biomarker Panel in Two Independent Sets of Resectable PDAC Plasma Samples

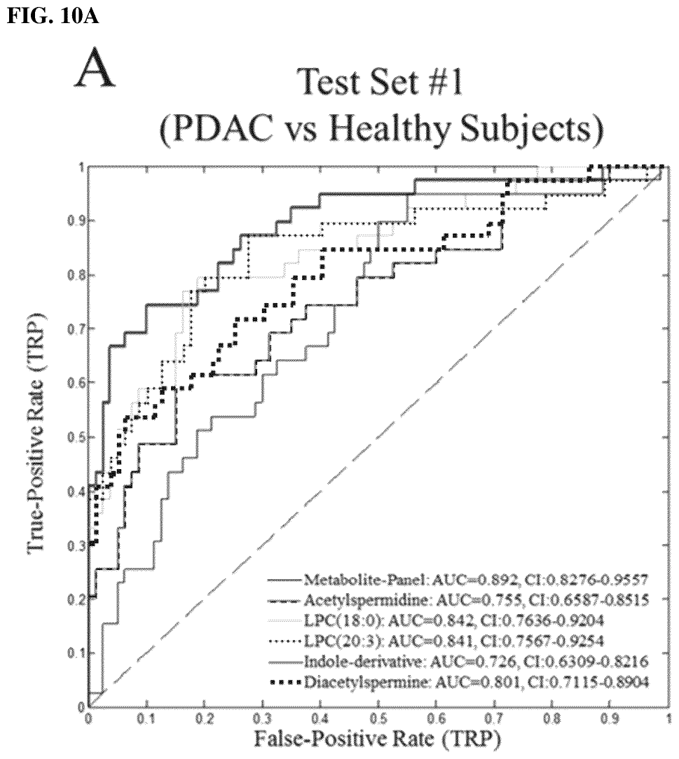

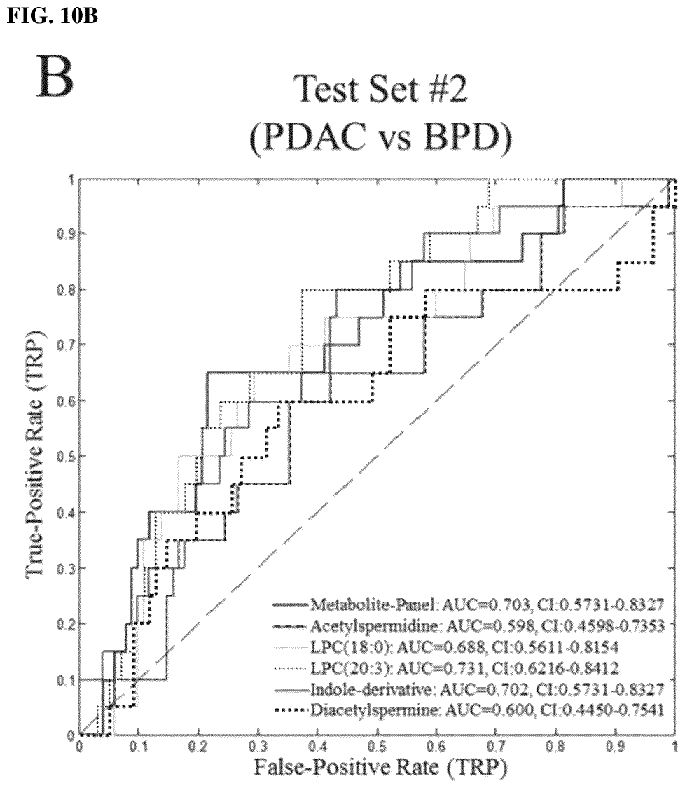

Blinded validation of the 5 metabolites individually and as a panel was performed in an independent set of plasma samples consisting of 39 resectable PDAC cases and 82 matched healthy controls (Test Set #1). All 5 biomarkers were significantly different (one-tailed p<0.001) in PDAC cases as compared to healthy controls with individual AUCs ranging from 0.73 to 0.84 Table 19). All 5 metabolites indicated the same direction of change (increased/decreased) as observed in the initial cohorts. The logistic regression model for the five-metabolite panel yielded an AUC of 0.89 (95% C.I.=0.828-0.956); exhibiting 67% sensitivity at 95% specificity (FIG. 10A and Table 19).

TABLE 18
Estimated coefficients for the 5-metabolite biomarker panel.
Coefficients were obtained by a logistic regression model
incorporating the logit link function.

| Estimated Coefficients: | Estimate | SE | tStat | pValue |
|---|---|---|---|---|
| (Intercept) | −2.2078 | 3.5256 | −0.6262 | 0.53118 |
| N8 | 2.1722 | 1.3954 | 1.5566 | 0.11955 |
| lysoPC180 | −6.768 | 5.9629 | −1.135 | 0.25637 |
| lysoPC203 | −1.4483 | 4.148 | −0.34916 | 0.72697 |
| indole3 | −0.17042 | 0.58436 | −0.29163 | 0.77057 |
| DAS | 3.5449 | 3.3304 | 1.0644 | 0.28715 |

39 observations; 33 error degrees of freedom
Dispersion: 1
Chi²-statistic vs. constant model: 18.2,
p-value = 0.00266

TABLE 19
Performance of individual metabolite markers and metabolite-panel in validation cohorts.

| | Test Set #1 | | | | | Test Set #2 | | | | |
|---|---|---|---|---|---|---|---|---|---|---|
| | | 95% | p- | | | | 95% | p- | | |
| Metabolite | AUC# | C.I.^{#} | value^{&} | Specificity* | Sensitivity** | AUC# | C.I.^{#} | value^{&} | Specificity* | Sensitivity** |
| Indole-derivative | 0.73 | 0.631- | <0.001 | 11 | 23 | 0.70 | 0.587- | <0.001 | 19 | 15 |
| | | 0.822 | | | | | 0.816 | | | |
| LPC (18:0) | 0.84 | 0.764- | <0.001 | 26 | 51 | 0.69 | 0.561- | 0.002 | 9 | 0 |
| | | 0.920 | | | | | 0.815 | | | |
| LPC (20:3) | 0.84 | 0.757- | <0.001 | 11 | 49 | 0.73 | 0.622- | <0.001 | 31 | 10 |
| | | 0.925 | | | | | 0.841 | | | |
| ACETYLSPERMIDINE | 0.76 | 0.659- | <0.001 | 28 | 33 | 0.60 | 0.460- | 0.083 | 1 | 5 |
| | | 0.852 | | | | | 0.735 | | | |
| DIACETYLSPERMINE | 0.80 | 0.712- | <0.001 | 28 | 51 | 0.60 | 0.445- | 0.104 | 0 | 5 |
| | | 0.890 | | | | | 0.754 | | | |
| 5-Marker Panel | 0.89 | 0.828- | <0.001 | 43 | 67 | 0.70 | 0.573- | 0.001 | 19 | 10 |
| | | 0.996 | | | | | 0.833 | | | |

#AUCs < 0.5 are flipped
*% specificity at 95% sensitivity
**% sensitivity at 95% specificity
^{&}one-tailed p-values for corresponding AUCs
Abbrev. LPC: lysophosphatidylcholine

The ability of the individual metabolites and panel to distinguish PDAC from BPD (low grade cysts) was tested in a second cohort (Test Set #2) consisting of 20 resectable PDAC and 102 subjects diagnosed with BPD derived from the same study as the confirmatory set (Set #2) but analyzed separately. Individual classification performances ranged from 0.60-0.73 (Table 2). The fixed logistic regression model for the five-metabolite panel yielded an AUC of 0.70 (95% C.I.=0.573-0.833); exhibiting 15% sensitivity at 95% specificity (FIG. 10B and Table 19).

### Combination of Metabolite- and Protein-Markers Improves Classification Performances

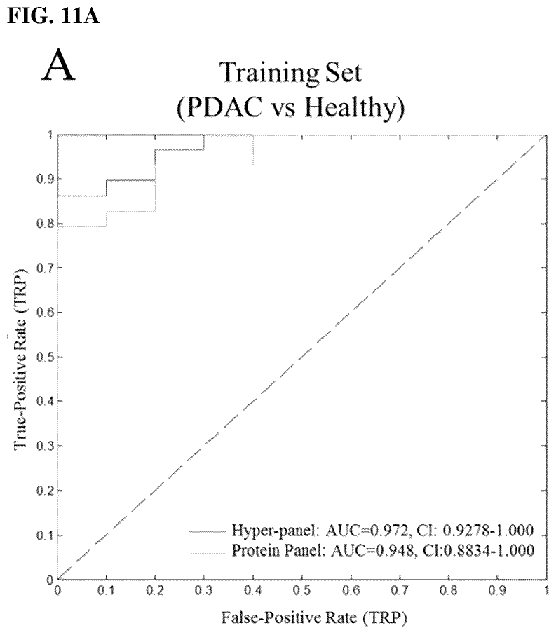

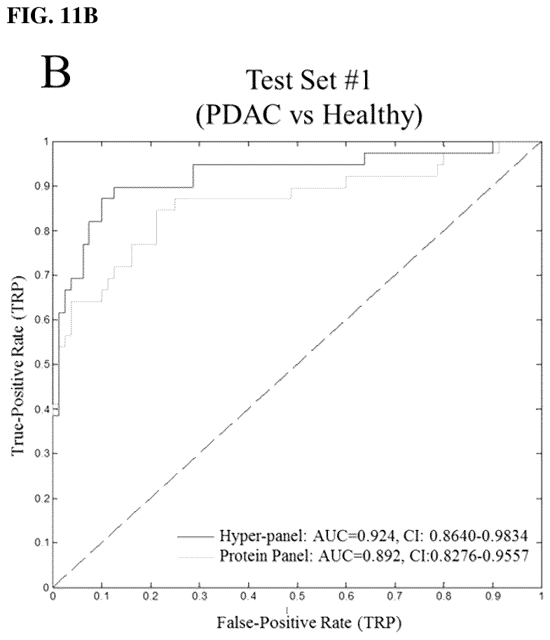

Previously, a protein-derived biomarker panel for early-stage PDAC was developed, which was validated in the same independent cohort (Test Set #1) described herein. It was therefore interrogated whether a hyper-panel consisting of the metabolite- and protein-panel would improve classification performance as compared to the protein-panel alone. The AUC of hyper-panel in the training set (29 PDAC versus 10 healthy controls) yielded an AUC of 0.97 with 95% CI (0.9278-1.000). The sensitivity of the metabolite panel alone for FPR values of 1% is estimated to be 0.6897. This estimate is improved statistically significantly to 0.8621 when considering the hyper-panel in the training set (corresponding one-tailed p-value=0.0390). Comparison of the protein panel (AUC=0.95) and the hyper-panel (AUC=0.97) in terms of AUCs in the training set, yielding a p-value of 0.1074 (FIG. 11A). The corresponding estimates during blinded validation (Test Set #1) for the protein panel yielded an AUC of 0.86 whereas the hyper-panel yielded an AUC of 0.92 with a corresponding one-tailed p-value for comparison equal to 0.0236 (FIG. 11B). This demonstrates an overall statistically significant improvement of the performance of the hyper-panel compared to the protein panel, indicating that the metabolite panel and the protein panel are complementary.

### PDAC Secrete Acetylated Polyamines

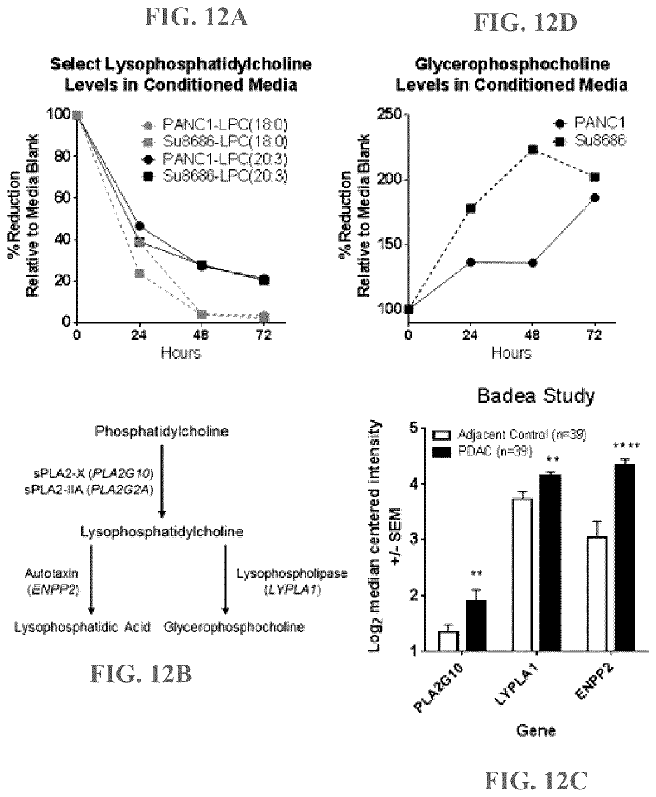

To determine whether elevations in plasma AcSperm and DAS were associated with disease status, cell lysates and serum-free conditioned media from 5 PDAC cell lines (CFPAC-1, MiaPaCa, SU8686, PANC03-27 and SW 1990) were analyzed. Metabolomic analysis of cell lysates revealed detectable levels of AcSperm and DAS in all 5 cell lines. Analysis of conditioned media indicated positive rates of AcSperm accumulation in all 5 cell lines whereas positive rates of DAS accumulation were observed in 3 of the 5 cell lines (FIG. 12A). Exploration of mRNA expression of polyamine-related enzymes in the Badea dataset indicated significant (paired T-test) PDAC-associated elevations in spermine synthase (SMS) and spermidine/spermine acetyltransferase (SAT1) as compared to adjacent control tissue whereas spermidine synthase (SRM), polyamine oxidase (PAOX) and spermine oxidase (SMOX) were significantly reduced (FIG. 11B), collectively suggesting increased acetylation of polyamines and subsequent secretion rather than their oxidation.

### PDAC Catabolize Extracellular Lysophosphatidylcholines

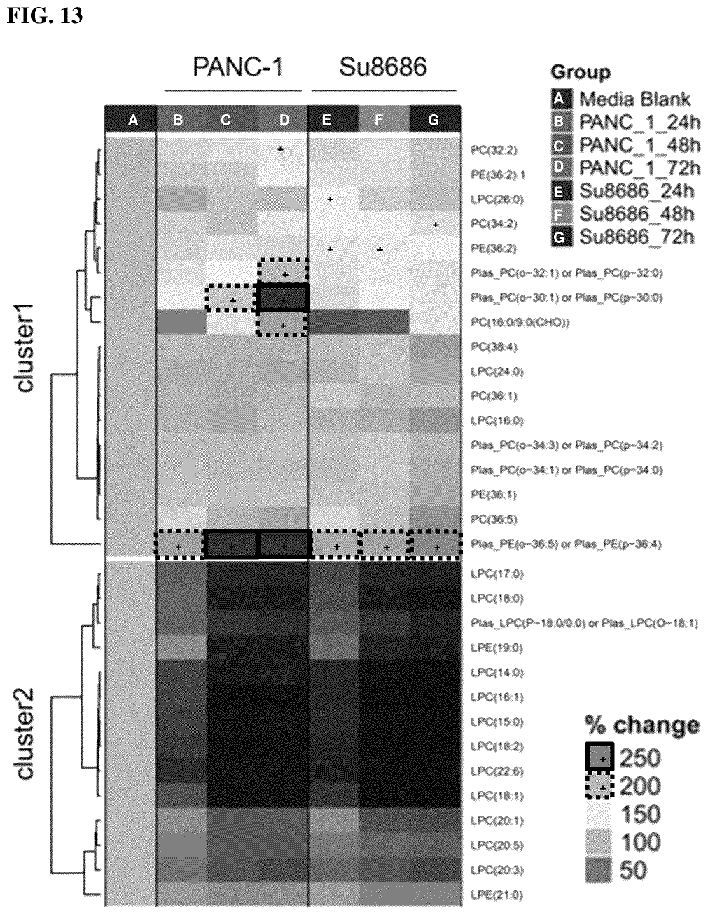

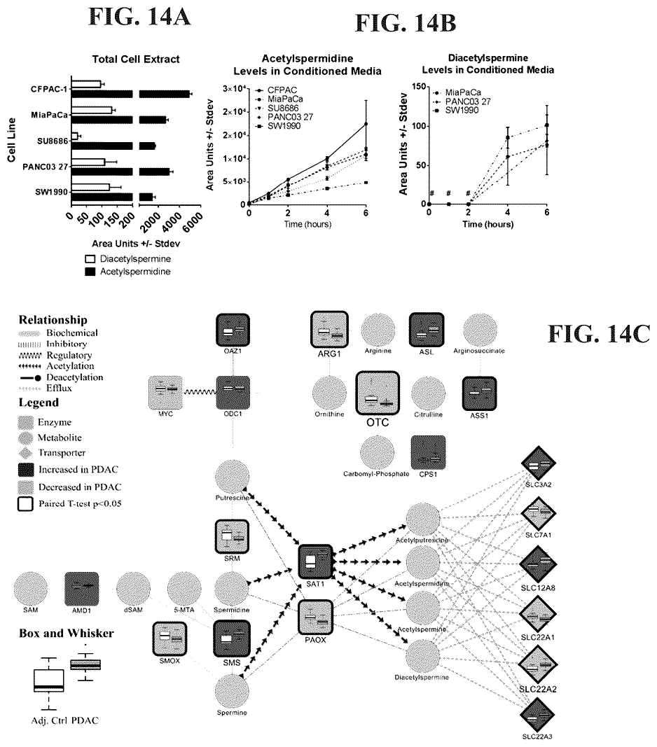

To determine whether PDAC cells catabolize/scavenge extracellular lipids, the lipid composition of serum-containing media from PANC1 and Su8686 cells was examined at 24, 48, and 72 hours post conditioning. The analysis indicated time-dependent reductions in several lysophospholipids (FIG. 13) including LPC (18:0) and LPC (20:3) (FIG. 12D). Concomitantly, glycerophosphocholine, a degradation product of LPCs, exhibited a time-dependent increase in conditioned media (FIG. 12B) collectively implicating active catabolism of extracellular LPCs. Evaluation of mRNA expression for enzymes involved in the catabolism of phospholipids and lysophospholipids (FIG. 12C) indicated significant (2-tailed Mann-Whitney U-test) PDAC-associated elevations in soluble phospholipase A2-X (PLA2G10), autotaxin (ENPP2) and lysophospholipase LYPLA1 relative to adjacent control tissue in the Badea dataset (FIG. 12D).

### Discussion

The primary objective of this study was to identify and validate a plasma metabolite-derived biomarker panel for resectable PDAC. Using an untargeted metabolomics approach, a 5-marker metabolite biomarker panel was identified and validated that is capable of distinguishing resectable PDAC cases from healthy individuals yielding an AUC of 0.89 in the validation cohort (Test Set #1). It was equally demonstrated that a hyper-panel consisting of the metabolite- and previously identified protein-panel significantly improves classification performances compared to the protein-panel alone (AUC: 0.92 vs 0.86; p: 0.024; Test Set #1) highlighting the complementary nature of the metabolite panel.

Given the low prevalence of PDAC, the multi-marker signature would be best suited for screening programs targeting high-risk subjects rather than the average risk population. These include individuals over age 50 years with new-onset diabetes mellitus, asymptomatic kindred of high-risk families, subjects with chronic pancreatitis, and patients incidentally diagnosed with mucin-secreting cysts of the pancreas. The metabolite-biomarker panel was able to significantly differentiate PDAC from low-grade pancreatic cyst in two separate sample sets, yielding AUC equal to 0.69 and 0.70 in the confirmation set and in test set #2, respectively.

Notably, no differences in plasma branched-chain amino acids (BCAA) were observed between cases and respective controls in contrast to previous findings. However, it should be noted that the predictive value of BCAAs were most prominent 2-5 years prior to diagnosis with levels returning towards baseline 0-2 years prior to diagnosis, consistent with observations of no differences in plasma BCAAs in samples taken at the time of diagnosis.

Altered polyamine metabolism has long been linked to tumorigenesis and hyper-proliferative disorders, being intimately involved in cell cycle progression. Polyamine synthesis is regulated by the rate-limiting enzymes ODC1 and AMD1 whereas their catabolism is regulated by SAT1. Previous findings indicated increased abundances of putrescine and AcSperm in pancreatic carcinomas as compared to histologically unaffected pancreas. Conversely, it was previously found that many polyamines including AcSperm were elevated in serum of cases as compared to healthy controls. These findings are in concordance with elevated mRNA expression of SAT1 in PDAC relative to adjacent control tissue in the Badea dataset and detection of AcSperm and DAS in cell lysates and their concurrent accumulation in conditioned media (FIG. 11). The findings described in this study, and those of others, indicate amplification of polyamine catabolism, a notion that is reflected in plasmas of subjects with PDAC. Notably, the elevation of DAS is not uniquely attributed to pancreatic cancer, inherently suggesting a more general role in its broader utility as a screening marker for cancer.

Previous studies indicated that plasma LPCs are significantly lower in PDAC relative to healthy controls or subjects with chronic pancreatitis, consistent with the findings of this study. The cell line data indicated that PDAC cells catabolize lysophospholipids, a notion that is supported by gene expression data in the Badea dataset (FIG. 12D), thereby providing plausibility for the observed reduction in plasma LPCs in PDAC subjects. Despite this, PDAC alone cannot explain the reduction in plasma LPC levels entirely, particularly in early stages of disease. Metastases to the liver, a crucial organ that regulates lipid metabolism, have been shown to occur at early stages of pancreatic cancer. Thus, it is plausible that reductions in plasma LPCs may be a reflection of both increased catabolism by cancerous cells as well as altered liver function that co-occurs with disease, a concept that will require additional investigation independent of the current study.

In conclusion, a metabolite-derived biomarker panel for early-stage PDAC was developed and validated that complements the previously identified protein-based biomarker panel.

### OTHER EMBODIMENTS

The detailed description set-forth above is provided to aid those skilled in the art in practicing the present disclosure. However, the disclosure described and claimed herein is not to be limited in scope by the specific embodiments disclosed herein because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1-81. (canceled)

82. A method comprising
obtaining a biological sample from a patient;
measuring the level of CA19-9 antigen in the biological sample;
measuring the level of TIMP1 antigen in the biological sample;
measuring the level of LRG1 antigen in the biological sample;
wherein the amount of CA19-9 antigen, TIMP1 antigen, and LRG1 antigen classifies the patient as being susceptible to pancreatic ductal adenocarcinoma (PDAC);
confirming the presence of PDAC using a diagnostic imaging technique; and
administering to the patient with PDAC a therapeutically effective amount of a treatment for the PDAC.

83. The method of claim 82, wherein the diagnostic imaging technique is chosen from computed tomography, endoscopic ultrasound, or endoscopic retrograde cholangiopancreatography.

84. The method of claim 82, wherein the levels of CA19-9 antigen, TIMP1 antigen, and LRG1 antigen are measured by:
contacting the sample with a first reporter molecule that binds CA19-9 antigen;
contacting the sample with a second reporter molecule that binds TIMP1 antigen; and
contacting the sample with a third reporter molecule that binds LRG1 antigen.

85. The method of claim 82, wherein the levels of CA19-9 antigen, TIMP1 antigen, and LRG1 antigen are measured by:
providing a surface with means for binding to CA19-9 antigen, TIMP1 antigen, and LRG1 antigen;
incubating the surface with the biological sample;
contacting the surface with a first reporter molecule that binds CA19-9 antigen;
contacting the surface with a second reporter molecule that binds TIMP1 antigen;
contacting the surface with a third reporter molecule that binds LRG1 antigen;
measuring the amount of the first reporter molecule that is associated with the surface;
measuring the amount of the second reporter molecule that is associated with the surface; and
measuring the amount of the third reporter molecule that is associated with the surface.

86. The method of claim 82, wherein the levels of CA19-9 antigen, TIMP1 antigen, and LRG1 antigen are measured by:
providing a surface with means for binding CA19-9 antigen, TIMP1 antigen, and LRG1 antigen;
incubating the surface with the biological sample;
contacting the surface with a first relay molecule that binds CA19-9 antigen;
contacting the surface with a second relay molecule that binds TIMP1 antigen;
contacting the surface with a third relay molecule that binds LRG1 antigen;
contacting the surface with a first reporter molecule that binds to the first relay molecule;
contacting the surface with a second reporter molecule that binds to the second relay molecule;
contacting the surface with a third reporter molecule that binds to the third relay molecule;
measuring the amount of the first reporter molecule that is associated with the first relay molecule and CA19-9 antigen;
measuring the amount of the second reporter molecule that is associated with the second relay molecule and TIMP1 antigen; and
measuring the amount of the third reporter molecule that is associated with the third relay molecule and LRG1 antigen.

87. The method of claim 82, wherein the levels of CA19-9 antigen, TIMP1 antigen, and LRG1 antigen are measured by:
providing a first surface with means for binding to CA19-9 antigen;
providing a second surface with means for binding to TIMP1 antigen;
providing a third surface with means for binding to LRG1 antigen;
incubating the first surface with the biological sample;
incubating the second surface with the biological sample;
incubating the third surface with the biological sample;
contacting the first surface with a first relay molecule that binds CA19-9 antigen;
contacting the second surface with a second relay molecule that binds TIMP1 antigen;
contacting the third surface with a third relay molecule that binds LRG1 antigen;
contacting the first surface with a first reporter molecule that binds to the first relay molecule;
contacting the second surface with a second reporter molecule that binds to the second relay molecule;
contacting the third surface with a third reporter molecule that binds to the third relay molecule;
measuring the amount of the first reporter molecule that is associated with the first relay molecule and CA19-9 antigen;
measuring the amount of the second reporter molecule that is associated with the second relay molecule and TIMP1 antigen; and
measuring the amount of the third reporter molecule that is associated with the third relay molecule and LRG1 antigen.

88. The method of claim 82, wherein the patient is classified as being susceptible to pancreatic ductal adenocarcinoma (PDAC) by performing a statistical analysis based on the amount of CA19-9 antigen, TIMP1 antigen, and LRG1 antigen present to determine a biomarker score with respect to PDAC.

89. The method of claim 88, wherein the levels of CA19-9 antigen, TIMP1 antigen, and LRG1 antigen are elevated in comparison to the levels of CA19-9 antigen, TIMP1 antigen, and LRG1 antigen in a reference patient or group that does not have PDAC.

90. The method of claim 89, wherein the reference patient or group is healthy.

91. The method of claim 89, wherein the levels of CA19-9 antigen, TIMP1 antigen, and LRG1 antigen are elevated in comparison to the levels of CA19-9 antigen, TIMP1 antigen, and LRG1 antigen in a reference patient or group that has chronic pancreatitis.

92. The method of claim 89, wherein the levels of CA19-9 antigen, TIMP1 antigen, and LRG1 antigen are elevated in comparison to the levels of CA19-9 antigen, TIMP1 antigen, and LRG1 antigen in a reference patient or group that has benign pancreatic disease.

93. The method of claim 82, wherein the subject is over age 50 years with new-onset diabetes mellitus, has chronic pancreatitis, has been incidentally diagnosed with mucin-secreting cysts of the pancreas, or is asymptomatic kindred of one of these high-risk groups.

94. The method of claim 82, further comprising administering at least one alternate diagnostic test for a patient assigned as having PDAC.

95. The method of claim 94, wherein the at least one alternate diagnostic test comprises an assay or sequencing of at least one ctDNA.

96. The method of claim 82, wherein the treatment is surgery, chemotherapy, radiation therapy, targeted therapy, or a combination thereof.

97. The method of claim 96, wherein the surgery is partial or complete surgical removal of cancerous tissue in the patient with PDAC.

98. The method of claim 97, wherein the surgery is chosen from a Whipple procedure, distal pancreatectomy, or total pancreatectomy.

99. The method of claim 96, wherein the radiation therapy is chosen from conventional/standard fraction radiation therapy or stereotactic body radiation.

100. The method of claim 99, wherein the chemotherapy treatment comprises the administration of a drug chosen from capecitabine (Xeloda), erlotinib (Tarceva), fluorouracil (5-FU), gemcitabine (Gemzar), irinotecan (Camptosar), leucovorin (Wellcovorin), nab-paclitaxel (Abraxane), nanoliposomal irinotecan (Onivyde), or oxaliplatin (Eloxatin).
